(19) United States
(12) Patent Application Publication
(10) Pub. No.: US 2025/0290056 A1
(43) Pub. Date: Sep. 18, 2025

(54) MODRNA-BASED CAS ENDONUCLEASE AND BASE EDITOR AND USES THEREOF

(71) Applicant: THE PENN STATE RESEARCH FOUNDATION, University Park, PA (US)

(72) Inventors: Xiaojun LIAN, University Park, PA (US); Tahir HAIDERI, University Park, PA (US); Alessandro HOWELLS, University Park, PA (US); Yuqian JIANG, University Park, PA (US)

(21) Appl. No.: 18/860,334

(22) PCT Filed: Apr. 27, 2023
(86) PCT No.: PCT/US2023/066305

§ 371 Date: Oct. 25, 2024

Related U.S. Application Data
(60) Provisional application No. 63/335,568, filed on Apr. 27, 2022.
(60) Provisional application No. 63/399,681, filed on Aug. 20, 2022.

(51) Int. Cl. C12N 9/22; A61K 39/00; C07K 14/005; C07K 14/82; C12N 15/10; C12N 15/11

(52) U.S. Cl. CPC C12N 9/226; C07K 14/005; C07K 14/82; C12N 15/102; C12N 15/111; A61K 39/00; A61K 2039/53; C12N 2310/20; C12N 2770/20022; C12N 2770/20034

(57) ABSTRACT

Disclosed herein are gene editing systems comprising chemically modified RNAs (modRNAs) encoding Cas endonucleases or base editors. Wherein a gene editing system comprising a first chemically modified RNA (modRNA) comprising a sequence encoding a CRISPRassociated (Cas) endonuclease or a base editor; and a second modRNA comprising a sequence encoding a mutated p53 protein, wherein the mutated p53 protein inhibits a wild type p53 protein from binding to a target domain thereof.

### CROSS REFERENCE TO RELATED APPLICATIONS

This application claims the benefit of U.S. Provisional Patent Application Ser. Nos. 63/335,568, filed on Apr. 27, 2022 and 63/399,681, filed on Aug. 20, 2022, which are expressly incorporated herein by reference in their entireties.

### STATEMENT REGARDING FEDERALLY SPONSORED RSEARCH

This invention was made with government support under Grant No. CBET1943696 awarded by the National Science Foundation. The Government has certain rights in the invention.

### BACKGROUND

CRISPR systems have revolutionized biomedical research because they offer an unprecedented opportunity for genome editing in human cells. However, how to deliver CRISPR effectors easily and efficiently remains to be explored. What are needed are systems and methods to delivery CRISPR systems into cells and uses thereof for gene editing.

### SUMMARY

Disclosed herein are chemically modified Cas sequences and base editor sequences for editing a target gene in a cell. It is shown herein the technology disclosed herein enables highly efficient gene editing and base editing. The modRNA delivery system for Cas or base editor is quicker and easier to perform. The modRNA delivered Cas endonuclease (e.g., a Cas9 endonuclease, a Cas6 endonuclease, or a Cas 13 endonuclease) or base editor editing efficiency is significantly higher than those delivered via traditional systems such as plasmids or viral vectors.

The details of one or more embodiments of the invention are set forth in the accompanying drawings and the description below. Other features, objects, and advantages of the invention will be apparent from the description and drawings, and from the claims.

In some aspects, disclosed herein is a gene editing system comprising a first chemically modified RNA (modRNA) comprising a sequence encoding a Cas endonuclease (e.g., a Cas9 endonuclease, a Cas6 endonuclease, or a Cas 13 endonuclease) or a base editor; and a second modRNA comprising a sequence encoding a mutated p53 protein. In some examples, the mutated p53 protein inhibits a wild type p53 protein from binding to a target domain thereof. In some embodiments, the first modRNA and the second modRNA each comprises a m7G (5) pppN1pN2p (CAP0) or m7G (5) pppN1mpNp (CAP1). In some embodiments, the first modRNA and/or the second modRNA each comprises one or more N1-methylpseudo-UTPs (N1meΨTP, or m1ΨTP). In some embodiments, the uracil residues in the modRNA sequences disclosed herein are all N1-methylpseudo-UTPs. In some embodiments, the first modRNA further comprises a 5′-unstranlated region (UTR) and/or a 3′-UTR. In some embodiments, the second modRNA further comprises a 5′-unstranlated region (UTR) and/or a 3′-UTR. In some embodiments, the first modRNA and the second modRNA each further comprises a polyA tail.

Accordingly, disclosed herein is a gene editing system comprising a first chemically modified RNA (modRNA) comprising a sequence encoding a Cas endonuclease (e.g., Cas9 endonuclease, a Cas6 endonuclease, or a Cas 13 endonuclease) or a base editor; and a second modRNA comprising a sequence encoding a mutated p53 protein, wherein the first modRNA and the second modRNA each comprises a m7G (5) pppN1pN2p (CAP0) or m7G (5) pppN1mpNp (CAP1), wherein the uridines of the first modRNA and/or the second modRNA are N1-methyl-pseudo-UTPs, and wherein the first modRNA and the second modRNA each further comprises a 5′-unstranlated region (UTR) and a 3′-UTR.

Also disclosed herein are gene editing systems of any preceding aspect further comprising a guide RNA (gRNA). Accordingly, in some aspects, disclosed herein is a gene editing system comprising a first chemically modified RNA (modRNA) comprising a sequence encoding a Cas endonuclease (e.g., a Cas9 endonuclease); a second modRNA comprising a sequence encoding a mutated p53 protein; and a gRNA. In some embodiments, the first modRNA comprises a sequence at least about 80% (at least about 80%, 85%, 90%, 95%, 98%, or 99%) identical to SEQ ID NO: In some embodiments, the second modRNA comprises a sequence at least about 80% (at least about 80%, 85%, 90%, 95%, 98%, or 99%) identical to SEQ ID NO: 2.

Also disclosed herein are gene editing systems of any preceding aspect comprising a first chemically modified RNA (modRNA) comprising a sequence encoding a sequence encoding a base editor; and a second modRNA comprising a sequence encoding a mutated p53 protein, wherein the mutated p53 protein inhibits a wild type p53 protein from binding to a target domain thereof. In some embodiments, the sequence encoding the base editor is at least about 80% (at least about 80%, 85%, 90%, 95%, 98%, or 99%) identical to SEQ ID NO: 3.

In some embodiments, the first modRNA and the second modRNA of any preceding aspect each further comprises a 5′-unstranlated region (UTR) and 3′-UTR. In some embodiments, the first modRNA and the second modRNA each further comprises a Woodchuck Hepatitis Virus (WHV) Posttranscriptional Regulatory Element (WPRE). In some embodiments, the WPRE locates at the 5′ end of the 3′-UTR. In some embodiments, the WPRE comprises a sequence at least about 80% (at least about 80%, 85%, 90%, 95%, 98%, or 99%) identical to SEQ ID NO: 4. Accordingly, in some aspects, disclosed herein is a gene editing system comprising a first chemically modified RNA (modRNA) comprising a sequence encoding a Cas endonuclease (e.g., a Cas9 endonuclease or Cas6 endonuclease) or a base editor; and a second modRNA comprising a sequence encoding a mutated p53 protein, wherein the first modRNA and the second modRNA each comprises a m7G (5) pppN1pN2p (CAP0) or m7G (5) pppN1mpNp (CAP1), wherein the uridines of the first modRNA and/or the second modRNA are N1-methyl-pseudo-UTPs, wherein the first modRNA and the second modRNA each further comprises a 5′-unstranlated region (UTR) and a 3′-UTR, wherein the first modRNA and the second modRNA each further comprises a Woodchuck Hepatitis Virus (WHV) Posttranscriptional Regulatory Element (WPRE).

Also disclosed herein are systems for expressing a polynucleotide sequence specifically in a cell, said system comprising: a regulatory RNA comprising a first nucleic acid sequence encoding a Cas6 endonuclease and a second nucleic acid sequence comprising a MicroRNA (miRNA) binding site, wherein miRNA is specifically expressed in the cell; and a gene-of-interest (GOI) RNA comprising a Cas6 targeting site and the polynucleotide sequence. In some embodiments, the regulatory RNA and/or the GIO RNA are chemically modified. In some embodiments, the chemically modified regulatory RNA and the chemically modified GIO RNA each comprises a m7G (5) pppN1pN2p (CAP0) or m7G (5) pppN1mpNp (CAP1). In some embodiments, the chemically modified regulatory RNA and the chemically modified GIO RNA each comprises a 5′-unstranlated region (UTR) and a 3′-UTR. In some embodiments, the chemically modified regulatory RNA and the chemically modified GIO RNA each comprises a polyA tail. In some embodiments, the chemically modified regulatory RNA and the chemically modified GIO RNA each comprises one or more N1-methylpseudo-UTPs (N1meΨTP, or m1ΨTP). In some embodiments, the uracil residues in the modRNA sequences disclosed herein are N1-methylpseudo-UTPs. In some embodiments, the first nucleic acid sequence encoding the Cas6 endonuclease is at least about 80% (about 80%, 85%, 90%, 95%, 98%, or 99%) identical to SEQ ID NO: 5. In some embodiments, the miRNA binding site is a miRNA-206 binding site, a mir-218 binding site, or a miR-375 binding site. In some embodiments, the miRNA binding site comprises a sequence at least about 80% (about 80%, 85%, 90%, 95%, 98%, or 99%) identical to SEQ ID NO: 6. In some embodiments, the Cas6 endonuclease binding site comprises a sequence at least about 80% (about 80%, 85%, 90%, 95%, 98%, or 99%) identical to SEQ ID NO: 7. In some embodiments, the system is for expressing a polynucleotide encodings a COVID spike protein in a skeletal muscle cell.

Also disclosed herein is a vaccine comprising the system of any preceding aspect. In some embodiments, the vaccine is a COVID vaccine. In some embodiments, vaccine comprises the system for expressing a polynucleotide encoding a COVID spike protein in a skeletal muscle cell.

Also disclosed herein is a method for expressing a polynucleotide sequence specifically in a cell, comprising introducing into the cell the system of any preceding aspects. In some embodiments, the cell is a neuronal cell, a skeletal muscle cell, or a pancreatic cell.

Also disclosed herein is a gene therapy composition comprising the gene editing system of any preceding aspect.

Also disclosed herein is a method of modifying a gene is a cell, comprising introducing into the cell the gene editing system of any preceding aspect.

Also disclosed herein is a treating a genetic disorder or myocardial infarctions (including, for example, muscular dystrophy, hereditary neuropathy, Parkinson's disease, X-Linked cardiomyopathy, or cancer) in a subject in need, comprising administering to the subject a therapeutically effective amount of the gene therapy composition of any preceding aspect.

### DESCRIPTION OF DRAWINGS

FIGS. 1A-1C show that Cas9 modRNA and sgRNA efficiently knock out an integrated GFP in hPSCs. (FIG. 1A) Schematic diagram for knocking out GFP in H1 OCT4-GFP cells using Cas9 modRNA and in-vitro synthesized sgRNA. (FIG. 1B) DNA templates used to synthesize modRNA for Cas9-2A-GFP, Cas9, Cas9-2A-Puro, p53DD, and ABE8e. Panel B is a summary of all the constructs used in the paper. (FIG. 1C) H1 OCT4-GFP cells were cultured on iMatrix-511 in mTeSR1 and transfected with different combinations of Cas9 modRNA and GFP sgRNA. On day 4, cells were collected and GFP expression was analyzed via flow cytometry. The percentage of GFP negative cells for each combination is shown in the form of a tiled heatmap. Experiments were repeated three times and representative data were shown.

FIGS. 2A-2E show that drug selection improved KO efficiency via Cas9Puro modRNA. (FIG. 2A) Schematic of sgRNA design targeting THY1 gene, encoding CD90 protein. (FIG. 2B) H9 cells were cultured on iMatrix-511 in mTeSR1 and transfected with Cas9Puro modRNA and either the CD90_1 or CD90_2 sgRNA. On day 4, cells were collected and CD90 expression was analyzed via flow cytometry. Representative flow cytometry results are shown for each target design. (FIG. 2C) H9 cells were cultured on iMatrix-511 in mTeSR1 and transfected with either 300 ng or 250 ng of Cas9Puro modRNA or 250 ng of Cas9 modRNA. Transfected cells underwent drug selection for 24 hours using puromycin beginning 12 hours after transfection. Following drug selection, cells were imaged (scale bar=200 μm) (FIG. 2D) and stained using TO-PRO 3 cell viability reagent before being counted using a flow cytometer (n=3; unpaired student's T-test). (FIG. 2E) H9 cells were transfected with 300 ng Cas9Puro modRNA, 100 ng CD90_1 sgRNA and underwent 24 hours of drug selection beginning 12 hours after transfection. On day 5, cells were collected and CD90 expression was analyzed by flow cytometry (n=3).

FIGS. 3A-3I show P53DD modRNA increased Cas9 modRNA mediated gene KO in hPSCs. (FIG. 3A) Schematic of optimal transfection protocol with the addition of p53DD modRNA. (FIG. 3B) Aggregated gene KO efficiencies across multiple replicates and batches in H1 OCT4-GFP cells, comparing results between transient plasmid DNA transfection and modRNA-based delivery with or without p53DD as well as RNP lipofection (plasmid: n=9; modRNA: n=20; plasmid+p53DD: n=6; modRNA+p53DD: n=13; RNP: n=3). (FIG. 3C) H9 cells cultured on iMatrix-511 in mTeSR1 were transiently transfected with either the plasmid DNA with or without p53DD plasmid, modRNA cocktail with or without p53DD modRNA, or Cas9 RNP. On day 5, cells were collected and CD90 expression was analyzed via flow cytometry. (FIG. 3D) Aggregated CD90 KO efficiencies across multiple replicates and batches in H9 cells, comparing results between transient plasmid DNA transfection and modRNA-based delivery without or with p53DD as well as RNP lipofection (plasmid: n=6; modRNA: n=8; plasmid+p53DD: n=6; modRNA+p53DD: n=14; RNP: n=3; one-way ANOVA with post-hoc Tukey's test). (E) Genotype of CD90KO H9 cells generated using CRISPR modRNA cocktail with p53DD modRNA (n=8). (FIG. 3F) G-banded karyotype analysis of CD90 KO H9 cells generated using modRNA cocktail with p53DD. (FIG. 3G) IMR90C4 cells cultured on iMatrix-511 in mTeSR1 were transfected with Cas9Puro modRNA, CD90 sgRNA, and p53DD modRNA. On day 5, cells were collected and CD90 expression was analyzed via flow cytometry (H9: n=14; IMR90C4: n=12). (FIGS. 3H and 3I) H1 OCT4-GFP cells were cultured on iMatrix-511 in mTeSR1 using a 12-well plate, and transfected with 1200 ng Cas9Puro modRNA, 200 ng CD90_1 sgRNA, 200 ng GFP sgRNA, and 200 ng p53DD modRNA. On day 5, cells were collected and GFP/CD90 expression was analyzed via flow cytometry (n=3). (FIG. 3H) Representative flow cytometry plot from day 5. (FIG. 3I) Quantification of flow cytometry results from day 5 cells. The sequences in FIG. 3E include SEQ ID NO: 16 (GCCTGCCTAGTGGACCAGAGCCTTCGTCTGGACTGCCGCCATG), SEQ ID NO: 17 (GCCTGCCTAGTGGACTGCCGCCATG), SEQ ID NO: 18 (GCCTGCCTAGTGGACCAGAGCCTTGAGAATTCTCATTGGACTGCCGCCATG), SEQ ID NO: 19 (GCCTGCCTAGTGGACCAGAGCCTTCTGGACTGCCGCCATG), SEQ ID NO: 20 (GCCTGCCTAGTGGACCAGAGCCATG), and SEQ ID NO: 21 (GCCTGCCTAGTGGACCAGAGCCTTCGTTCTGGACTGCCGCCATG).

FIGS. 4A-4D show that ModRNA ABESe is more efficient over plasmid-based method. (FIG. 4A) Schematic of mechanism for gene KO via base editing. The dCas9 guides the fused ABE8e to the specific genomic region to perform the desired base edit. This desired base edit mutates the splice acceptor or donor region, so that after transcription, the spliceosome fails to splice out the intron or splices an exon, respectively. (FIG. 4B) Representative flow cytometry plots of cell population that were transfected with ABE8e+sgRNA, which were delivered in plasmid DNA or modRNA form. Cell populations were stained with a conjugated anti-B2M-APC antibody. (FIG. 4C) Quantification of B2M negative cells following either plasmid DNA or modRNA ABE8e transfection (n=3; one-way ANOVA with post-hoc Tukey's test). (FIG. 4D) Sequencing result of the B2M intron 1 splice donor site within a single cell clonal line. This indicates that one allele had two A:T to G:C base edits (both within the ABE8e editing window) and the other allele received only the desired splice donor base edit (base edits shown in red font). The sequences in FIG. 4D include SEQ ID NO: 22 (GGCTATCCAGCGTGAGTCTCTCC), SEQ ID NO: 23 (GGCTATCCAGCGcGAGcCTCTCC), and SEQ ID NO: 24 (GGCTATCCAGCGcGAGTCTCTCC).

FIGS. 5A-5D show optimization of modRNA delivery for CRISPR mediated gene editing in hPSCs. (FIG. 5A) Representative gating strategy used for excluding dead cells and subsequent doublet discrimination for excluding high fluorescence events during flow cytometry analysis (FIG. 5B) H1 and H9 cells were cultured on iMatrix-511 in mTeSR1 using a 24-well plate and transfected with Cas9GFP modRNA using Lipofectamine Stem Transfection Reagent (1:2 ratio). 24 hours later GFP expression was analyzed by flow cytometry. (FIGS. 5C and 5D) H1 OCT4-GFP cells were cultured on iMatrix-511 in mTeSR1 and transfected with different combinations of Cas9 modRNA and GFP sgRNA. On day 4, cells were collected and GFP expression was analyzed via flow cytometry. (FIG. 5C) Flow cytometry plots for combinations of 750 ng, 500 ng, and 250 ng Cas9 modRNA with either 750 ng, 500 ng, 250 ng, or 100 ng GFP sgRNA. (FIG. 5D) Flow cytometry plots for combinations of 250 ng and 125 ng Cas9 modRNA with either 100 ng, 50 ng, or 10 ng GFP sgRNA. Experiment was repeated three times and representative data were shown.

FIGS. 6A-6D show delivery of Cas9Puro modRNA in hPSCs. (FIG. 6A) CD90 expression in untransfected H9 cells. (FIG. 6B) Representative image of H9 cells on DO prior to transfection with Cas9Puro modRNA (scale bar=200 μm). (FIG. 6C) Representative gating strategy for counting live cells after staining with TO-PRO 3 cell viability reagent. (FIG. 6D) Representative flow cytometry plots for data summarized in FIG. 2D.

FIGS. 7A-7J show Cas9 mediated gene KO with p53DD in hPSCs. (FIG. 7A) Representative flow cytometry plots of GFP KO in H1 OCT4-GFP cells transiently transfected with plasmid DNA or modRNA with or without p53DD as well as RNP method. (FIG. 7B) Representative flow cytometry plot of RNP mediated CD90 KO in H9 cells on day 5 post-transfection. (FIG. 7C) Representative flow plots of β-catenin KO in H9 cells transiently transfected with either plasmid DNA of modRNA with or without p53DD. Cells were collected on day 5 post-transfection and β-catenin expression was analyzed via flow cytometry. (FIG. 7D) Representative flow cytometry plot of RNP mediated β-catenin KO in H9 cells on day 5 post-transfection. (FIG. 7E) Aggregated β-catenin KO efficiencies across multiple replicates and batches in H9 cells, comparing results between transient transfection of plasmid DNA and modRNA-based delivery with or without p53DD as well as RNP lipofection (Plasmid: n=6; modRNA: n=6; plasmid+p53DD: n=6; modRNA+p53DD: n=6; RNP: n=6; one-way ANOVA with post-hoc Tukey's test). (FIG. 7F) H9 cells were cultured in iMatrix-511 with mTeSR1 and transfected with either Cas9 modRNA and CD90 sgRNA or Cas9 protein and CD90 sgRNA. On day 2, cells were collected and stained with TO-PRO 3 cell viability reagent before being counted using a flow cytometer (n=3; unpaired student's T-test). (FIG. 7G) Flow cytometry analyses of CD90 expression in untransfected and transfected H9 cells with Cas9 modRNA and CD90 sgRNA. (FIG. 7H) Off-target analysis of CD90 KO H9 cells generated using CRISPR modRNA cocktail with p53DD. (FIG. 7I) Representative flow cytometry plot of CD90 KO in IMR90C4 cells using CRISPR modRNA cocktail with p53DD. (FIG. 7J) IMR90C4 cells cultured on iMatrix-511 in mTeSR1 were transfected with Cas9Puro modRNA, CTNNB1 sgRNA, and p53DD modRNA. On day 5, cells were collected and β-catenin expression was analyzed via flow cytometry. Representative flow cytometry plot and quantification (H9: n=6; IMR90C4: n=3). The sequences in 25 FIG. 7H include SEQ ID NO: (TGAATGACACCATGCAGCCCCGCCCATGGGCCCTCGTCTGGACTGCCTCTTTC), SEQ ID NO: 26 (CAGAGCTGCAGTGCAGACGAGGGTTGGGCACCTCAGAGCTGCAGTGCAGACAG), and SEQ ID NO: 27 (CTTTTTATATGTTGTGTCTCTGATGATTTTCCCTCTTCTGGACTGCCGCATAG)

FIGS. 8A-8B show that both cap 1 and cap 0 modRNA structures can mediate efficient genome editing in hPSCs. IMR90C4 cells were cultured on iMatrix-511 in mTeSR1 using a 12-well plate and transfected with 600 ng Cas9Puro modRNA (Cap0 or Cap1), 200 ng CD90 sgRNA, and 200 ng p53DD modRNA. On day 5, cells were collected and CD90 expression was analyzed by flow cytometry. (FIG. 8A) Representative flow cytometry plot. (FIG. 8B) Quantification of flow cytometry results from day 5 cells (n=3).

FIG. 9 shows plasmid map and sequence for XLoneV3-ABE8e.

FIGS. 10A-10D show tdTomato modRNA expression in the rat brain (24 h).

FIG. 11 shows cell type-specific modRNA system.

FIG. 12 displays a schematic showing Cas9+p53DD mediated gene knockout.

FIG. 13 displays a schematic showing base editing mediated gene knockout.

FIG. 14 shows the comparison between modRNA versus plasmid DNA.

FIG. 15 shows schematic of ABE8e-P2A-EGFP plasmid.

FIG. 16 shows AB38e modRNA used to knock out gene B2M in hPSCs.

FIG. 17 shows modRNA used to knock out gene B2M in hPSCs.

FIGS. 18A-8C show tissue Specific Expression of ModR-GOI in stem cell-derived skeletal muscle cells. FIG. 18A shows modified mRNA (modRNA) constructs designed to express the GOI in only skeletal muscle cells. FIG. 18B shows flow cytometry of undifferentiated stem cells transfected with either ModR-GOI or co-transfected with ModR-GOI and ModR-miR206a. FIG. 18C shows immunoflourescent staining of MF-20 showing colocalization of tdTomato and the skeletal muscle cells derived from stem cells.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### Definitions

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Reference will now be made in detail to the embodiments of the invention, examples of which are illustrated in the drawings and the examples. This invention may, however, be embodied in many different forms and should not be construed as limited to the embodiments set forth herein.

Ranges can be expressed herein as from “about” one particular value, and/or to “about” another particular value. By “about” is meant within 10% of the value, e.g., within 9, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA and/or RNA-DNA that is single- or double-stranded, optionally comprising synthetic, non-natural, or altered nucleotide bases. On occasion double-stranded DNA will be referred to “duplex DNA” or “dsDNA”. Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

The term “genome” as it applies to a prokaryotic and eukaryotic cell or organism cells encompasses not only chromosomal DNA found within the nucleus, but organelle DNA found within subcellular components (e.g., mitochondria, or plastid) of the cell.

“Open reading frame” is abbreviated ORF.

As used herein, “homologous recombination” (HR) includes the exchange of

DNA fragments between two DNA molecules at the sites of homology. The frequency of homologous recombination is influenced by a number of factors. Different organisms vary with respect to the amount of homologous recombination and the relative proportion of homologous to non-homologous recombination. Generally, the length of the region of homology affects the frequency of homologous recombination events; the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al., (1982) Cell 31:25-33; Shen and Huang, (1986) Genetics 112:441-57; Watt et al., (1985) Proc. Natl. Acad. Sci. USA 82:4768-72, Sugawara and Haber, (1992) Mol Cell Biol 12:563-75, Rubnitz and Subramani, (1984) o/Cell Biol 4:2253-8; Ayares et al., (1986) Proc. Natl. Acad. Sci. USA 83:5199-203; Liskay et al., (1987) Genetics 115:161-7.

“Sequence identity” or “identity” in the context of nucleic acid or polypeptide sequences refers to the nucleic acid bases or amino acid residues in two sequences that are the same when aligned for maximum correspondence over a specified comparison window.

The term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90%, or 95%, or any percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, WI). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

“BLAST” is a searching algorithm provided by the National Center for Biotechnology Information (NCBI) used to find regions of similarity between biological sequences. The program compares nucleotide or protein sequences to sequence databases and calculates the statistical significance of matches to identify sequences having sufficient similarity to a query sequence such that the similarity would not be predicted to have occurred randomly. BLAST reports the identified sequences and their local alignment to the query sequence. It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any percentage from 50% to 100%. Indeed, any amino acid identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

Polynucleotide and polypeptide sequences, variants thereof, and the structural relationships of these sequences can be described by the terms “homology”, “homologous”, “substantially identical”, “substantially similar” and” corresponding substantially” which are used interchangeably herein. These refer to polypeptide or nucleic acid sequences wherein changes in one or more amino acids or nucleotide bases do not affect the function of the molecule, such as the ability to mediate gene expression or to produce a certain phenotype. These terms also refer to modification(s) of nucleic acid sequences that do not substantially alter the functional properties of the resulting nucleic acid relative to the initial, unmodified nucleic acid. These modifications include deletion, substitution, and/or insertion of one or more nucleotides in the nucleic acid fragment. Substantially similar nucleic acid sequences encompassed may be defined by their ability to hybridize (under moderately stringent conditions, e.g., 0.5×SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms to highly similar fragments, such as genes that duplicate functional enzymes from closely related organisms. Post-hybridization washes determine stringency conditions.

The term “fragment” refers to a contiguous set of nucleotides or amino acids. In one embodiment, a fragment is 2, 3, 4, 5, 6, 7 8, 9, 10, 11, 12, 13, 14, 15, 16, 17, 18, 19, 20, or greater than 20 contiguous nucleotides. In one embodiment, a fragment is 2, 3, 4, 5, 6, 7 8, 9, 10, 11, 12, 13, 14, 15, 16, 17, 18, 19, 20, or greater than 20 contiguous amino acids. A fragment may or may not exhibit the function of a sequence sharing some percent identity over the length of said fragment.

The terms “fragment that is functionally equivalent” and “functionally equivalent fragment” are used interchangeably herein. These terms refer to a portion or subsequence of an isolated nucleic acid fragment or polypeptide that displays the same activity or function as the longer sequence from which it derives. In one example, the fragment retains the ability to alter gene expression or produce a certain phenotype whether or not the fragment encodes an active protein. For example, the fragment can be used in the design of genes to produce the desired phenotype in a modified plant. Genes can be designed for use in suppression by linking a nucleic acid fragment, whether or not it encodes an active enzyme, in the sense or antisense orientation relative to a plant promoter sequence.

“Gene” includes a nucleic acid fragment that expresses a functional molecule such as, but not limited to, a specific protein, including regulatory sequences preceding (5′ noncoding sequences) and following (3′ non-coding sequences) the coding sequence. “Native gene” refers to a gene as found in its natural endogenous location with its own regulatory sequences.

“Coding sequence” refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures.

A “mutated gene” is a gene that has been altered through human intervention. Such a “mutated gene” has a sequence that differs from the sequence of the corresponding non-mutated gene by at least one nucleotide addition, deletion, or substitution.

As used herein, a “targeted mutation” is a mutation in a gene (referred to as the target gene), including a native gene, that was made by altering a target sequence within the target gene using any method known to one skilled in the art, including a method involving a guided Cas endonuclease system as disclosed herein.

By “domain” it is meant a contiguous stretch of nucleotides (that can be RNA, DNA, and/or RNA-DNA-combination sequence) or amino acids.

The term “conserved domain” or “motif” means a set of polynucleotides or amino acids conserved at specific positions along an aligned sequence of evolutionarily related proteins. While amino acids at other positions can vary between homologous proteins, amino acids that are highly conserved at specific positions indicate amino acids that are essential to the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family.

A “codon-modified gene” or “codon-preferred gene” or “codon-optimized gene” is a gene having its frequency of codon usage designed to mimic the frequency of preferred codon usage of the host cell.

An “optimized” polynucleotide is a sequence that has been optimized for improved expression in a particular heterologous host cell.

A “promoter” is a region of DNA involved in recognition and binding of RNA polymerase and other proteins to initiate transcription. The promoter sequence consists of proximal and more distal upstream elements, the latter elements often referred to as enhancers.

An “enhancer” is a DNA sequence that can stimulate promoter activity and may be an innate element of the promoter or a heterologous element inserted to enhance the level or tissue-specificity of a promoter. Promoters may be derived in their entirety from a native gene or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

Promoters that cause a gene to be expressed in most cell types at most times are commonly referred to as “constitutive promoters”. The term “inducible promoter” refers to a promoter that selectively express a coding sequence or functional RNA in response to the presence of an endogenous or exogenous stimulus, for example by chemical compounds (chemical inducers) or in response to environmental, hormonal, chemical, and/or developmental signals. Inducible or regulated promoters include, for example, promoters induced or regulated by light, heat, stress, flooding or drought, salt stress, osmotic stress, phytohormones, wounding, or chemicals such as ethanol, abscisic acid (ABA), jasmonate, salicylic acid, or safeners.

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al, (1989) Plant Cell 1:671-680.

“RNA transcript” refers to the product resulting from RNA polymerase-catalyzed transcription of a DNA sequence. When the RNA transcript is a perfect complimentary copy of the DNA sequence, it is referred to as the primary transcript or pre-mRNA. An RNA transcript is referred to as the mature RNA or mRNA when it is a RNA sequence derived from posttranscriptional processing of the primary transcript pre-mRNA. “Messenger RNA” or “mRNA” refers to the RNA that is without introns and that can be translated into protein by the cell.

“cDNA” refers to a DNA that is complementary to, and synthesized from, an mRNA template using the enzyme reverse transcriptase. The cDNA can be single-stranded or converted into double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (see, e.g., U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “genome” refers to the entire complement of genetic material (genes and non-coding sequences) that is present in each cell of an organism, or virus or organelle; and/or a complete set of chromosomes inherited as a (haploid) unit from one parent.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., the coding sequence is under the transcriptional control of the promoter). Coding sequences can be operably linked to regulatory sequences in a sense or antisense orientation. In another example, the complementary RNA regions can be operably linked, either directly or indirectly, 5′ to the target mRNA, or 3′ to the target mRNA, or within the target mRNA, or a first complementary region is 5′ and its complement is 3′ to the target mRNA.

Generally, “host” refers to an organism or cell into which a heterologous component (polynucleotide, polypeptide, other molecule, cell) has been introduced. As used herein, a “host cell” refers to an in vivo or in vitro eukaryotic cell, prokaryotic cell (e.g., bacterial or archaeal cell), or cell from a multicellular organism (e.g., a cell line) cultured as a unicellular entity, into which a heterologous polynucleotide or polypeptide has been introduced. In some embodiments, the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, an insect cell, a mammalian cell, a pig cell, a cow cell, a goat cell, a sheep cell, a rodent cell, a rat cell, a mouse cell, a non-human primate cell, and a human cell. In some cases, the cell is in vitro. In some cases, the cell is in vivo.

The term “recombinant” refers to an artificial combination of two otherwise separated segments of sequence, e.g., by chemical synthesis, or manipulation of isolated segments of nucleic acids by genetic engineering techniques.

The terms “plasmid”, “vector” and “cassette” refer to a linear or circular extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell.

“Transformation cassette” refers to a specific vector comprising a gene and having elements in addition to the gene that facilitates transformation of a particular host cell. “Expression cassette” refers to a specific vector comprising a gene and having elements in addition to the gene that allow for expression of that gene in a host.

The term “expression”, as used herein, refers to the production of a functional end-product (e.g., an mRNA, guide RNA, or a protein) in either precursor or mature form.

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327:167-170; International Publication Number WO2007025097, published Mar. 1, 2007, which is incorporated herein in its entirety). A CRISPR locus can consist of a CRISPR array, comprising short direct repeats (CRISPR repeats) separated by short variable DNA sequences (called spacers), which can be flanked by diverse Cas (CRISPR-associated) genes.

The term “Cas protein” refers to a polypeptide encoded by a Cas (CRISPR-associated) gene. A Cas protein includes proteins encoded by a gene in a Cas locus and includes adaptation molecules as well as interference molecules. An interference molecule of a bacterial adaptive immunity complex includes endonucleases. A Cas endonuclease described herein comprises one or more nuclease domains. A Cas endonuclease includes but is not limited to: the novel Cas-alpha protein disclosed herein, a Cas9 protein, a Cas12a (Cpf1) protein, a Cas12b (C2c1) protein, a Cas13a (C2c2) protein, a Cas12c (C2c3) protein, Cas3, Cas3-HD, Cas 5, Cas7, Cas8, Cas10, or combinations or complexes of these. A Cas protein may be a “Cas endonuclease” or “Cas effector protein”, that when in complex with a suitable polynucleotide component, is capable of recognizing, binding to, and optionally nicking or cleaving all or part of a specific polynucleotide target sequence. The Cas-alpha endonucleases of the disclosure include those having one or more RuvC nuclease domains. A Cas protein is further defined as a functional fragment or functional variant of a native Cas protein, or a protein that shares at least 30%, between 30% and 35%, at least 35%, between 35% and 40%, at least 40%, between 40% and 45%, at least 45%, between 45% and 50%, at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native Cas protein, and retains at least partial activity of the native sequence.

A Cas endonuclease may also include a multifunctional Cas endonuclease. The term “multifunctional Cas endonuclease” and “multifunctional Cas endonuclease polypeptide” are used interchangeably herein and includes reference to a single polypeptide that has Cas endonuclease functionality (comprising at least one protein domain that can act as a Cas endonuclease) and at least one other functionality, such as but not limited to, the functionality to form a complex (comprises at least a second protein domain that can form a complex with other proteins). In one aspect, the multifunctional Cas endonuclease comprises at least one additional protein domain relative (either internally, upstream (5′), downstream (3′), or both internally 5′ and 3′, or any combination thereof) to those domains typical of a Cas endonuclease.

The terms “Cascade” and “Cascade complex” are used interchangeably herein and include reference to a multi-subunit protein complex that can assemble with a polynucleotide forming a polynucleotide-protein complex (PNP). Cascade is a PNP that relies on the polynucleotide for complex assembly and stability, and for the identification of target nucleic acid sequences. Cascade functions as a surveillance complex that finds and optionally binds target nucleic acids that are complementary to a variable targeting domain of the guide polynucleotide.

The terms “5′-cap” and “7-methylguanylate (m7G) cap” are used interchangeably herein. A 7-methylguanylate residue is located on the 5′ terminus of messenger RNA (mRNA) in eukaryotes. RNA polymerase II (Pol II) transcribes mRNA in eukaryotes. Messenger RNA capping occurs generally as follows: the most terminal 5′ phosphate group of the mRNA transcript is removed by RNA terminal phosphatase, leaving two terminal phosphates. A guanosine monophosphate (GMP) is added to the terminal phosphate of the transcript by a guanylyl transferase, leaving a 5 ‘-5’ triphosphate-linked guanine at the transcript terminus. Finally, the 7-nitrogen of this terminal guanine is methylated by a methyl transferase.

The terms “functional fragment”, “fragment that is functionally equivalent” and “functionally equivalent fragment” of a guide RNA, crRNA or tracrRNA are used interchangeably herein and refer to a portion or subsequence of the guide RNA, crRNA or tracrRNA, respectively, of the present disclosure in which the ability to function as a guide RNA, crRNA or tracrRNA, respectively, is retained.

The terms “functional variant”, “variant that is functionally equivalent” and “functionally equivalent variant” of a guide RNA, crRNA or tracrRNA (respectively) are used interchangeably herein, and refer to a variant of the guide RNA, crRNA or tracrRNA, respectively, of the present disclosure in which the ability to function as a guide RNA, crRNA or tracrRNA, respectively, is retained.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, optionally bind to, and optionally nick or cleave (introduce a single or double-strand break) the DNA target site.

The term “Cas endonuclease recognition domain” or “CER domain” (of a guide polynucleotide) is used interchangeably herein and includes a nucleotide sequence that interacts with a Cas endonuclease polypeptide. A CER domain comprises a (trans-acting) tracrNucleotide mate sequence followed by a tracrNucleotide sequence. The CER domain can be composed of a DNA sequence, a RNA sequence, a modified DNA sequence, a modified RNA sequence (see for example EIS20150059010A1, published 26 Feb. 2015), or any combination thereof.

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double-strand break) the DNA target site.

The terms “target site”, “target sequence”, “target site sequence,” target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, a locus, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, (iv) a chemical alteration of at least one nucleotide, or (v) any combination of (i)-(iv).

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, (iv) a chemical alteration of at least one nucleotide, or (v) any combination of (i)-(iv).

Methods for “modifying a target site” and “altering a target site” are used interchangeably herein and refer to methods for producing an altered target site.

As used herein, the term “before”, in reference to a sequence position, refers to an occurrence of one sequence upstream, or 5′, to another sequence.

Efficiency is a measure of enzyme activity relative to the theoretical limit of diffusion-limited substrate binding to the enzyme (Johnson et al. 2019). Herein the term “efficiency” is used to refer to the steady-state kinetic parameter, kcat/Km, which is the apparent second-order rate constant for substrate binding and conversion to product. Kinetic parameters derived using direct methods as described in Gong et al. 2018, Liu et al. 2020, and Bravo et al, 2022 (herein incorporated by reference in their entirety) are implicitly given. Even though WT Cas9 catalyzes only a single enzyme turnover such that the products of the reaction remain tightly bound to the enzyme, the equations defining kcat/Km are still valid and will be a function of each step in the reaction cycle from substrate binding to the first largely irreversible step. For example, a mutant Cas9 molecule can have about a 50-fold or less, 40-fold or less, 30-fold or less, 20-fold or less, 10-fold or less, 9-fold or less, 8-fold or less, 7-fold or less, 6-fold or less, 5-fold or less, 4-fold or less, 3-fold or less, 2-fold or less, or 1-fold or less decrease in efficiency as compared to its non-mutant (native) counterpart or to another Cas9. A mutant Cas9 can also have a 1-fold or more, 2-fold or more, 3-fold or more, 4-fold or more, 5-fold or more, 6-fold or more, 7-fold or more, 8-fold or more, 9-fold or more, 10-fold or more, 20-fold or more, 30-fold or more, 40-fold or more, or 50-fold or more increase in efficiency as compared to its non-mutant (native) counterpart or another Cas9.

By “specificity” is meant a function of the efficiency of reaction for a desired substrate relative to that for an undesired substrate (Johnson et al. 2019; Liu et al. 2020; Liu et al., 2019; Gong et al. 2018). Mathematically, efficiency is defined as the ratio of kcat/Km values to the two substrates. For Cas9, specificity is defined as (kcat/Km) on-target-DNA/(kcat/Km) off-target-DNA. For example, a mutant Cas9 molecule can have about a 50-fold or less, 40-fold or less, 30-fold or less, 20-fold or less, 10-fold or less, 9-fold or less, 8-fold or less, 7-fold or less, 6-fold or less, 5-fold or less, 4-fold or less, 3-fold or less, 2-fold or less, or 1-fold or less decrease in specificity as compared to its non-mutant (native) counterpart or to another Cas9. A mutant Cas9 can also have a 1-fold or more, 2-fold or more, 3-fold or more, 4-fold or more, 5-fold or more, 6-fold or more, 7-fold or more, 8-fold or more, 9-fold or more, 10-fold or more, 20-fold or more, 30-fold or more, 40-fold or more, or 50-fold or more increase in specificity as compared to its non-mutant (native) counterpart or another Cas9.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The term “nucleic acid editing domain,” as used herein refers to a protein or enzyme capable of making one or more modifications (e.g., deamination of a cytidine residue) to a nucleic acid (e.g., DNA or RNA). Exemplary nucleic acid editing domains include, but are not limited to a deaminase, a nuclease, a nickase, a recombinase, a methyltransferase, a methylase, an acetylase, an acetyltransferase, a transcriptional activator, or a transcriptional repressor domain. In some embodiments the nucleic acid editing domain comprises a deaminase (e.g., a cytidine deaminase or an adenine deaminase).

An “adenine deaminase” is an enzyme involved in purine metabolism. It is needed for the breakdown of adenosine from food and for the turnover of nucleic acids in tissues. Its primary function in humans is the development and maintenance of the immune system. An adenine deaminase catalyzes hydrolytic deamination of adenosine (forming inosine, which base pairs as G) in the context of DNA.

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

Nucleotide: The fundamental unit of nucleic acid molecules. A nucleotide includes a nitrogen-containing base attached to a pentose monosaccharide with one, two, or three phosphate groups attached by ester linkages to the saccharide moiety. The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U).

As used throughout, by a “subject” (or a “host”) is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. Administration of the therapeutic agents can be carried out at dosages and for periods of time effective for treatment of a subject.

“Therapeutically effective amount” or “therapeutically effective dose” of a composition refers to an amount that is effective to achieve a desired therapeutic result. In some embodiments, a desired therapeutic result is reduction or clearance of a pathogen. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, cells, salts, esters, amides, proagents, active metabolites, isomers, fragments, analogs, and the like. When the term “therapeutic agent” is used, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially. Prophylactic treatments are administered to a subject prior to onset (e.g., before obvious signs of a cardiac disorder), during early onset (e.g., upon initial signs and symptoms of a cardiac disorder), or after an established development of a cardiac disorder. Prophylactic administration can occur for several days to years prior to the manifestation of symptoms of a disorder

The term “polypeptide” refers to a compound made up of a single chain of D- or L-amino acids or a mixture of D- and L-amino acids joined by peptide bonds.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “polynucleotide” refers to a single or double stranded polymer composed of nucleotide monomers.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

### Gene Editing Systems

Disclosed herein is a gene editing system that comprises a chemically modified RNA (modRNA) comprising a sequence encoding a Cas endonuclease (e.g., a Cas9 endonuclease, a Cas6 endonuclease, or a Cas 13 endonuclease) or a base editor. It is shown herein that a p53 dominant negative protein (p53DD protein) can greatly increase modRNA-based genome editing efficiency. Accordingly, the gene editing system disclosed herein further comprises a modRNA comprising a sequence encoding a mutated p53 protein, wherein the mutated p53 protein inhibits a wild type p53 protein from binding to a target domain thereof. Accordingly, in some aspects, disclosed herein is a gene editing system comprising a first chemically modified RNA (modRNA) comprising a sequence encoding a Cas endonuclease (e.g., a Cas9 endonuclease, a Cas6 endonuclease, or a Cas 13 endonuclease); and a second modRNA comprising a sequence encoding a mutated p53 protein. In some examples, the mutated p53 protein inhibits a wild type p53 protein from binding to a target domain thereof. In some embodiments, the first modRNA and the second modRNA each comprises a m7G (5) pppN1p N2p (CAP0) or m7G (5) pppN1mpNp (CAP1).

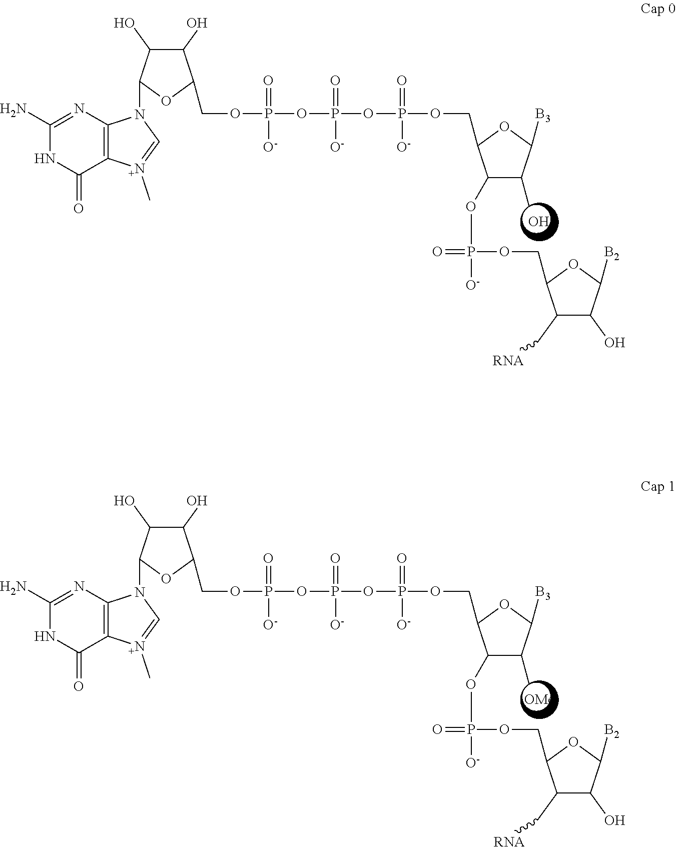

The systems disclosed herein may further comprise a guide RNA (gRNA). Accordingly, in some aspects, disclosed herein is a gene editing system comprising a first chemically modified RNA (modRNA) comprising a sequence encoding a Cas endonuclease (e.g., a Cas9 endonuclease, a Cas6 endonuclease, or a Cas 13 endonuclease); a second modRNA comprising a sequence encoding a mutated p53 protein; and a gRNA. In some embodiments, the first modRNA comprises a sequence at least about 80% (about 80%, 85%, 90%, 95%, 98%, or 99%) identical to SEQ ID NO: 1. In some embodiments, the second modRNA comprises a sequence at least about 80% (about 80%, 85%, 90%, 95%, 98%, or 99%) identical to SEQ ID NO: 2.

In some embodiments, the base editor comprises a catalytically inactive Cas9 endonuclease. It should be understood and herein contemplated that dead Cas9 (dCas9) is an enzymatically inactive mutant of Cas9 in which its endonuclease activity is nonfunctional. Base editing is a CRISPR/Cas—mediated precise editing to generate single nucleotide changes in DNA or RNA. The cytosine and adenine base editors (CBE and ABE) are catalytically impaired deaminases that replace a C-G to T-A and A-T to G-C mutation, respectively. In RNA, the RNA base editor can replace adenine to inosine. Fusion of base editors to either dCas9 does not create a double-strand break. Thus, base editors offer precise genome editing with much cleaner product output. In some examples, the base editor disclosed herein is covalently tethered to a dead CRISPR/Cas9 domain (dCas9). The dCas9 enables researchers to target a specific adenosine: thymidine base pair within an organism's genome in order to localize the adenosine deaminase to perform the adenosine to inosine reaction.

Accordingly, in some aspects, disclosed herein is a gene editing system comprising a first chemically modified RNA (modRNA) comprising a sequence encoding a base editor; and a second modRNA comprising a sequence encoding a mutated p53 protein, wherein the mutated p53 protein inhibits a wild type p53 protein from binding to a target domain thereof. In some embodiments, the sequence encoding the base editor is at least about 80% (at least about 80%, 85%, 90%, 95%, 98%, or 99%) identical to SEQ ID NO: 3. In some embodiments, the base editor is a cytosine base editor or an adenine base editor.

In some embodiments, the first modRNA and the second modRNA can each further comprises a 5′-unstranlated region (UTR) and 3′-UTR. In some embodiments, the first modRNA further comprises a 5′-unstranlated region (UTR) and/or a 3′-UTR. In some embodiments, the second modRNA further comprises a 5′-unstranlated region (UTR) and/or a 3′-UTR. In some embodiments, the modRNA disclosed herein comprises a Woodchuck Hepatitis Virus (WHV) Posttranscriptional Regulatory Element (WPRE). In some embodiments, the WPRE locates at the 5′ end of the 3′-UTR of the modRNA. The WPRE can further increase the stability of the modRNA. In some embodiments, the first modRNA and/or the second modRNA each comprises one or more N1-methylpseudo-UTPs (N1meΨTP, or m1ΨTP). In some embodiments, the uracil residues in the modRNA sequences disclosed herein are N1-methylpseudo-UTPs. In some embodiments, the first modRNA and the second modRNA each further comprises a polyA tail.

Accordingly, in some aspects, disclosed herein is a gene editing system comprising a first chemically modified RNA (modRNA) comprising a sequence encoding a base editor; and a second modRNA comprising a sequence encoding a mutated p53 protein, wherein the mutated p53 protein inhibits a wild type p53 protein from binding to a target domain thereof, and wherein the first modRNA and the second modRNA each further comprises a Woodchuck Hepatitis Virus (WHV) Posttranscriptional Regulatory Element (WPRE). In some embodiments, the WPRE comprises a sequence at least about 80% (about 80%, 85%, 90%, 95%, 98%, or 99%) identical to SEQ ID NO:

In some aspects, the gene editing system disclosed herein comprises one or more of DNA sequences disclosed herein or the RNA equivalents thereof.

Also disclosed herein is a gene therapy composition comprising the gene editing system disclosed herein. In some aspects, the gene therapy composition comprises a gene editing system comprising a first chemically modified RNA (modRNA) comprising a sequence encoding a Cas endonuclease (e.g., a Cas9 endonuclease, a Cas6 endonuclease, or a Cas 13 endonuclease) or a base editor; and a second modRNA comprising a sequence encoding a mutated p53 protein. In some embodiments, the gene therapy composition or the gene editing system disclosed herein further comprises a gRNA. In some embodiments, the modRNA comprises a Woodchuck Hepatitis Virus (WHV) Posttranscriptional Regulatory Element (WPRE). In some embodiments, the WPRE locates at the 5′ end of the 3′-UTR of the modRNA.

How to design novel modRNA constructs which enables translation of modRNA exclusively in one cell type but not others is a fundamental question which is critical for both basic science research and therapeutic applications. Disclosed herein is a cell-type-specific modRNA expression via miRNAs and CRISPR-Cas6. Cas6 (also known as Casy4), a previously identified endoribonuclease present in a wide range of prokaryotes with the CRISPR-Cas system, binds and cleaves within the repeat sequences that separate the individual invader targeting elements in the CRISPR locus transcript.

Accordingly, in some aspects, disclosed herein is a system for expressing a polynucleotide sequence specifically in a cell, said system comprising: a regulatory RNA comprising a first nucleic acid sequence encoding a Cas6 endonuclease and a second nucleic acid sequence comprising a MicroRNA (miRNA) binding site, wherein miRNA is specifically expressed in the cell; and a gene-of-interest (GOI) RNA comprising a Cas6 endonuclease targeting site and the polynucleotide sequence. In some embodiments, the regulatory RNA and/or the GIO RNA are chemically modified. In some embodiments, the chemically modified regulatory RNA and the chemically modified GIO RNA each comprises a m7G (5) pppN1pN2p (CAP0) or m7G (5) pppN1mpNp (CAP1). In some embodiments, the first nucleic acid sequence encoding the Cas6 endonuclease is at least about 80% (at least about 80%, 85%, 90%, 95%, 98%, or 99%) identical to SEQ ID NO: 5. In some embodiments, the chemically modified regulatory RNA and/or the chemically modified GIO RNA each comprises one or more N1-methylpseudo-UTPs (N1meΨTP, or m1ΨTP). In some embodiments, the uracil residues in the modRNA sequences disclosed herein are N1-methylpseudo-UTPs. In some embodiments, the first modRNA and the second modRNA each further comprises a 5′-unstranlated region (UTR) and a 3′-UTR. In some embodiments, the first modRNA and the second modRNA each further comprises a poly A tail.

It is herein contemplated that miR-206, miR-218, and miR-375 binding site can be used to restrict expression only in skeletal muscle cells, motor neuron, and pancreatic islet cells, respectively. Accordingly, in some embodiments, the miRNA binding site is a miRNA-206 binding site, a mir-218 binding site, or a miR-375 binding site. In some embodiments, the miRNA binding site comprises a sequence at least about 80% (at least about 80%, 85%, 90%, 95%, 98%, or 99%) identical to SEQ ID NO: 6, 30, or 32.

In some embodiments, the Cas6 endonuclease binding site is at least about 5 nucleotides in length (including, for example, at least 6, 7, 8, 9, 10, 11, 12, 13, 14, 15, 16, 17, 18, 19, 20, 21, 22, 23, 24, 25, 256, 27, 28, 29, 30, 32, 34, 36, 38, 40, 42, 44, 46, 48, 50, 60, 70, 80, 90, or 100 nucleotides in length). In some embodiments, the Cas6 endonuclease binding site is at least about 28 nucleotides in length. In some embodiments, the Cas6 endonuclease binding site comprises a sequence at least about 80% (at least about 80%, 85%, 90%, 95%, 98%, or 99%) identical to SEQ ID NO: 7.

The system disclosed herein can be used for expressing a polynucleotide in a cell (including, for example, a neuronal cell, a pancreatic cell, or a skeletal muscle cell). In some embodiments, the system disclosed herein can be used for expressing a polynucleotide encoding a protein of a pathogen (including, for example, a viral protein or a bacterial protein) or a mammalian protein in a cell. In some embodiments, the system is for expressing a polynucleotide encoding a viral protein in a skeletal muscle cell. In some embodiments, the system is for expressing a polynucleotide encoding an influenza protein or a SARS-CoV-2 protein in a skeletal muscle cell. In some embodiments, the SARS-CoV-2 protein is a SARS-CoV-2 spike protein. In some embodiments, the system disclosed herein can be used for expressing a polynucleotide in a cell, wherein the polynucleotide encodes a human protein. The system herein can be used for expressing a normal human protein in a cell (including, for example, a neuronal cell, a pancreatic cell, or a skeletal muscle cell) in a subject in need (e.g., a subject having a genetic disorder) for treatment of a genetic disorder.

In some embodiments, the regulatory RNA and the GOI RNA each further comprises a 5′-unstranlated region (UTR) and 3′-UTR. In some embodiments, the regulatory RNA and the GOI RNA each further comprises a Woodchuck Hepatitis Virus (WHV) Posttranscriptional Regulatory Element (WPRE). In some embodiments, the WPRE locates at the 5′ end of the 3′-UTR. Accordingly, in some aspects, disclosed herein is a gene editing system comprising a first chemically modified RNA (modRNA) comprising a sequence encoding a Cas endonuclease (e.g., a Cas9 endonuclease or Cas6 endonuclease) or a base editor; and a second modRNA comprising a sequence encoding a mutated p53 protein, wherein the first modRNA and the second modRNA each comprises a m7G (5) pppN1pN2p (CAP0) or m7G (5) pppN1mpNp (CAP1), wherein the uridines of the first modRNA and/or the second modRNA are N1-methyl-pseudo-UTPs, wherein the first modRNA and the second modRNA each further comprises a 5′-unstranlated region (UTR) and a 3′-UTR, wherein the first modRNA and the second modRNA each further comprises a Woodchuck Hepatitis Virus (WHV) Posttranscriptional Regulatory Element (WPRE).

The cell-type-specific expression system disclosed herein can utilized in a vaccine. In some aspects, disclosed herein is a vaccine comprising any of the systems disclosed herein.

Also disclosed herein is an expression vector comprising one or more nucleic acid sequences encoding the one or more modRNAs disclosed herein.

Also disclosed herein is an engineered cell comprising any of the systems disclosed herein.

### Methods

The systems disclosed herein show high efficiency in delivering Cas editing gene in a cell (e.g., a hematopoietic stem cell). Accordingly, in some aspects, disclosed herein is a method of modifying a gene in a cell, comprising introducing into the cell any of the gene editing systems disclosed herein.

In some embodiments, the gene editing system comprising a first chemically modified RNA (modRNA) comprising a sequence encoding a Cas endonuclease (e.g., a Cas9 endonuclease, a Cas6 endonuclease, or a Cas 13 endonuclease) or a base editor; and a second modRNA comprising a sequence encoding a mutated p53 protein. In some examples, the mutated p53 protein inhibits a wild type p53 protein from binding to a target domain thereof. In some embodiments, the first modRNA and the second modRNA each comprises a m7G (5) pppN1p N2p (CAP0) or m7G (5) pppN1mpNp (CAP1).

The system can further comprise a guide RNA (gRNA). Accordingly, in some aspects, disclosed herein is a method of modifying a gene in a cell, comprising introducing into the cell the gene editing system, wherein the gene editing system comprises a first chemically modified RNA (modRNA) comprising a sequence encoding a Cas endonuclease (e.g., a Cas9 endonuclease or a Cas6 endonuclease) or a base editor; a second modRNA comprising a sequence encoding a mutated p53 protein; and a gRNA. In some embodiments, the first modRNA comprises a sequence at least about 80% (about 80%, 85%, 90%, 95%, 98%, or 99%) identical to SEQ ID NO: 1. In some embodiments, the second modRNA comprises a sequence at least about 80% (at least about 80%, 85%, 90%, 95%, 98%, or 99%) identical to SEQ ID NO: 2.

Also disclosed herein is a method of modifying a gene in a cell, comprising introducing into the cell the gene editing system, wherein the gene editing system comprises a first chemically modified RNA (modRNA) comprising a sequence encoding a base editor; and a second modRNA comprising a sequence encoding a mutated p53 protein, wherein the mutated p53 protein inhibits a wild type p53 protein from binding to a target domain thereof. In some embodiments, the sequence encoding the base editor is at least about 80% (at least about 80%, 85%, 90%, 95%, 98%, or 99%) identical to SEQ ID NO: 3.

In some embodiments, the first modRNA and the second modRNA disclosed herein can each further comprises a 5′-unstranlated region (UTR) and 3′-UTR. In some embodiments, the first modRNA and the second modRNA each further comprises a Woodchuck Hepatitis Virus (WHV) Posttranscriptional Regulatory Element (WPRE). In some embodiments, the WPRE locates at the 5′ end of the 3′-UTR. In some embodiments, the WPRE comprises a sequence at least about 80% (at least about 80%, 85%, 90%, 95%, 98%, or 99%) identical to SEQ ID NO: 4. Also disclosed herein is a method for expressing a polynucleotide sequence specifically in a cell, comprising introducing into the cell any of the systems disclosed herein. In some embodiments, the cell is a neuronal cell, a skeletal muscle cell, or a pancreatic cell. In some embodiments, the system comprises a regulatory RNA comprising a first nucleic acid sequence encoding a Cas6 endonuclease and a second nucleic acid sequence comprising a microRNA (miRNA) binding site, wherein miRNA is specifically expressed in the cell; and a gene-of-interest (GOI) RNA comprising a Cas6 endonuclease targeting site and the polynucleotide sequence. In some embodiments, the regulatory RNA and/or the GIO RNA are chemically modified. In some embodiments, the chemically modified regulatory RNA and the chemically modified GIO RNA each comprises a m7G (5) pppN1pN2p (CAP0) or m7G (5) pppN1mpNp (CAP1). In some embodiments, the chemically modified regulatory RNA and the chemically modified GIO RNA sequence each comprises one or more N1-methylpseudo-UTPs (N1meΨTP, or m1ΨTP). In some embodiments, the uracil residues in the modRNA sequences disclosed herein are N1-methylpseudo-UTPs. In some embodiments, the chemically modified regulatory RNA and the chemically modified GIO RNA sequence each comprises a 5′-unstranlated region (UTR) and a 3′-UTR. In some embodiments, the chemically modified regulatory RNA and the chemically modified GIO RNA each comprises a polyA tail. In some embodiments, the first nucleic acid sequence encoding the Cas6 endonuclease is at least about 80% (at least about 80%, 85%, 90%, 95%, 98%, or 99%) identical to SEQ ID NO: 5.

In some embodiments, the Cas6 endonuclease binding site is at least about 5 nucleotides in length (including, for example, at least 6, 7, 8, 9, 10, 11, 12, 13, 14, 15, 16, 17, 18, 19, 20, 21, 22, 23, 24, 25, 256, 27, 28, 29, 30, 32, 34, 36, 38, 40, 42, 44, 46, 48, 50, 60, 70, 80, 90, or 100 nucleotides in length). In some embodiments, the Cas6 endonuclease binding site is at least about 28 nucleotides in length. In some embodiments, the Cas6 endonuclease binding site comprises a sequence at least about 80% (at least about 80%, 85%, 90%, 95%, 98%, or 99%) identical to SEQ ID NO: 7.

In some embodiments, the regulatory RNA and the GOI RNA each further comprises a 5′-unstranlated region (UTR) and 3′-UTR. In some embodiments, the regulatory RNA and the GOI RNA each further comprises a Woodchuck Hepatitis Virus (WHV) Posttranscriptional Regulatory Element (WPRE). In some embodiments, the WPRE locates at the 5′ end of the 3′-UTR.

### Nucleic Acid Delivery

In the methods described above which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega Biotec, Inc., Madison, WI), as well as other liposomes developed according to procedures standard in the art. In addition, the disclosed nucleic acid or vector can be delivered in vivo by electroporation, the technology for which is available from Genetronics, Inc. (San Diego, CA) as well as by means of a SONOPORATION machine (ImaRx Pharmaceutical Corp., Tucson, AZ).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the antibody-encoding nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established. Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. For additional discussion of suitable formulations and various routes of administration of therapeutic compounds, see, e.g., Remington: The Science and Practice of Pharmacy (19th ed.) ed. A. R. Gennaro, Mack Publishing Company, Easton, PA 1995.

In some aspects, disclosed herein is a method of treating a genetic disorder (including, for example, muscular dystrophy, hereditary neuropathy, Parkinson's disease, X-Linked cardiomyopathy, or cancer) or myocardial infarctions in a subject in need, comprising administering to the subject a therapeutically effective amount of the gene therapy composition or the gene editing system. In some examples, the gene therapy composition or the gene editing system disclosed herein can be used to treat Parkinson's disease by targeting genes, including, for example, glucosylceramidase beta (GBA), phospholipase A2 group VI (PLA2G6), F-box 7 (FBX07), Leucine rich repeat kinase 2 (LRRK2), Parkinsonism associated deglycase or Parkinson disease protein 7 (PARK7), Phosphatase and tensin homolog (PTEN)-induced kinase 1 (PINK1), Parkin (PRKN), Vacuolar protein sorting ortholog 35 (VPS35), microtubule associated protein tau (MAPT), GTP cyclohydrolase I gene (GCH1), Cyclin G Associated Kinase (GAK), Bone marrow stromal cell antigen I (BST1), Synaptotagmin-11 (SYT11), Human lymphocyte antigen DRB5 (HLA-DRB5), or Alpha-synuclein (aSyn) gene (SNCA), or a transcript thereof. In some examples, the gene therapy composition or the gene editing system disclosed herein can be used to treat muscular dystrophy or X-Linked cardiomyopathy by targeting gene Duchenne Muscular dystrophy gene (DMD). In some examples, the gene therapy composition or the gene editing system disclosed herein can be used to treat Hereditary neuropathy by targeting gene peripheral myelin protein 22 (PMP22). In some examples, the gene therapy composition or the gene editing system disclosed herein can be used to treat cancer by targeting a pro-oncogene and/or an oncogene (including, for example, ABL, BCL1, CDK4, EGFR, ERBB2 (NEU), HSTF1, INT1/WNT1, INT2, MDM2, MET, MYB, MYC (c-MYC), MYCN, MYCL1, MYCLK1, PTEN, Beclin-1, DAPK, RAF1 (c-RAF), HRAS1, KRAS, KRAS2, NRAS, REL, HST, KS3, HER1/EGFR, VGFR, HER2/NEU, HER3, FMS, KIT, ROS, ALK, RET, TRK, MAS, FGR, FES, SRC, YES1, ETS2, AM11, ERBA1, ERBA2, FAS, JUN, NMYC, LMYC, MYB, SK1, TS1, BCL2, BCL-XL, c-FLIP, p53, MDM2, HER-2, BCR/ABL). In some embodiments, the genetic disorder is a cancer.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

In one aspect, the disclosed methods can be employed 30, 20, 10, 9, 8, 7, 6, 5, 4, 3, 2 years, 12, 11, 10, 9, 8, 7, 6, 5, 4, 3, 2 months, 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2 hours, 60, 45, 30, 15, 10, 9, 8, 7, 6, 5, 4, 3, 2, or 1 minute prior to onset of a disorder; or 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 days, 3, 4, 5, 6, 7, 8, 9, 10, 11, 12 months, 2, 3, 4, 5, 6, 7, 8, 9, 10 or more years after onset of a disorder.

### Route of Administration

Genome editing systems, or cells altered or manipulated using such systems, which include the Cas endonuclease disclosed herein, can be administered to subjects by any suitable mode or route, whether local or systemic. Systemic modes of administration include oral and parenteral routes. Parenteral routes include, by way of example, intravenous, intramarrow, intrarterial, intramuscular, intradermal, subcutaneous, intranasal, and intraperitoneal routes. Components administered systemically may be modified or formulated to target, e.g., HSCs, hematopoietic stem/progenitor cells, or erythroid progenitors or precursor cells.

Local modes of administration include, by way of example, intramarrow injection into the trabecular bone or intrafemoral injection into the marrow space, and infusion into the portal vein. In an embodiment, significantly smaller amounts of the components (compared with systemic approaches) may exert an effect when administered locally (for example, directly into the bone marrow) compared to when administered systemically (for example, intravenously). Local modes of administration can reduce or eliminate the incidence of potentially toxic side effects that may occur when therapeutically effective amounts of a component are administered systemically.

Administration may be provided as a periodic bolus (for example, intravenously) or as continuous infusion from an internal reservoir or from an external reservoir (for example, from an intravenous bag or implantable pump). Components may be administered locally, for example, by continuous release from a sustained release drug delivery device.

### EXAMPLES

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. While the invention has been described with reference to particular embodiments and implementations, it will be understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. Such equivalents are intended to be encompassed by the following claims. It is intended that the invention not be limited to the particular implementations disclosed herein, but that the invention will include all implementations falling within the scope of the appended claims.

### Example 1: Robust Genome Editing Via modRNA-Based Cas9 or Base Editor in Human Pluripotent Stem Cell

Herein, the study developed modified mRNA (modRNA) based CRIPSR systems that utilized Cas9 and p53DD modRNA or base editor (ABE8e) modRNA for the purposes of knocking out genes in hPSCs via simple lipid-based transfection. ABE8e modRNA was employed to disrupt the splice donor site, resulting defective splicing of the target transcript, and ultimately leading to gene knockout. Using the modRNA delivery system, 73.3%±11.2% and 69.6±3.8% knockout efficiency was achieved with Cas9 and p53DD modRNA, and ABE8e modRNA, respectively, which was significantly higher than the plasmid-based systems. In summary, this study demonstrates that the non-integrating modRNA based CRISPR methods hold great promise as a more efficient and accessible technique for genome editing of hPSCs.

### Introduction

CRISPR-Cas systems are widely used for genome editing in a wide variety of cell types and are especially useful for high-throughput genome-wide screens. Cas9 is the most used endonuclease of the CRISPR-Cas family and can precisely cleave genomic DNA via double-stranded breaks (DSBs) when paired with a programmable single guide RNA (sgRNA) with minimal off-target effects. Repair of DSBs can occur through one of the two intrinsic pathways in mammalian cells: non-homologous end joining (NHEJ) and homology-directed repair (HDR). NHEJ results in insertions or deletions (indels) which can lead to frameshift mutations and consequently, gene knockout (KO). Alternatively, co-delivery of a donor DNA template can precisely introduce desired sequence edits via the HDR pathway. DNA cleavage is mediated by the HNH and RuvC domains of the Cas9 protein. Mutations in these domains result in a catalytically inactive Cas9 (dCas9), which allows for a more general platform for RNA-guided, genomic delivery of a wide variety of covalently tethered effector proteins, among them being base editors. The two primary base editors used in practice are based on either adenosine or cytidine deaminases. They are also known as adenine base editors (ABEs) or cytidine base editors (CBEs). ABEs specifically convert deoxyadenosine (dA) to deoxyinosine (dI), which in turn, the dI gets repaired to deoxyguanosine (dG). CBEs, on the other hand, convert deoxycytidine (dC) to deoxyuridine (dU), which gets repaired to deoxythymidine (dT). When the adenosine or cytidine deaminase is covalently tethered to a dCas9, this enables researchers to introduce a genomic point mutation at high fidelity without DSBs, thus significantly reducing the risk of potentially detrimental indels and chromosomal rearrangements at off-target sites. ABEs and CBEs have been leveraged to correct disease-related point mutations and for gene KO purposes, at relatively high efficiencies and specificities.

Human pluripotent stem cells (hPSCs) can be expanded almost indefinitely while still maintaining their ability to differentiate into all somatic cell lineages. They can be utilized to generate in vitro cell culture models for studying human development and disease modeling when coupled with CRISPR-Cas9 systems. Despite their remarkable potential, the current state-of-the-art methods for delivering CRISPR components into hPSCs are far from ideal. Virus-mediated gene delivery is considered an efficient method for the delivery of CRISPR components into most cell types. Commonly used viral vectors include lentiviruses, adeno-associated viruses (AAVs), and adenoviruses. Lentiviruses are normally integrating, which can increase the risk of tumorigenicity, and therefore, hPSC lines with lentiviral integrations can be counterproductive during their use in cell-based therapies. Additionally, hPSCs were reported to be resistant to lentiviral infection due to unique intrinsic immunity. AAVs and adenoviruses are two non-integrating alternatives to lentiviruses. However, adenoviruses are known to trigger high levels of innate immune response in transduced cells which can lead to inflammation. AAVs have a relatively low packaging limit (˜4.7 kb), making it difficult to deliver CRISPR components. Additionally, AAVs and adenoviruses are laborious to produce and require the use of specialized equipment for their purification.

Non-viral state-of-the-art methods for delivering CRISPR components into hPSCs include a variety of physical and chemical delivery strategies. Electroporation and lipid nanoparticles (LNPs) are two commonly used non-viral delivery methods, which use plasmid DNA for the delivery of CRISPR components via either nucleofection or transfection reagents. These methods however have low transfection efficiency and can be cytotoxic to cells. Ribonucleoproteins (RNPs), on the other hand, consisting of Cas9 protein complexed with a sgRNA, have also been shown to efficiently edit the genome. Cas9 protein is commercially available, however, CBEs and ABEs are not, and producing purified samples of these proteins can be cumbersome and not feasible for many labs.

An emerging alternative to these above approaches is the use of chemically modified RNA (modRNA) for the delivery of CRISPR effectors into cells. ModRNA is coined ‘modified’ because chemically modified nucleotides are used during in vitro transcription synthesis. It has been shown that when un-modified regular mRNA is introduced to mammalian cells, it is not stable, and it triggers the cellular immune response. However, modRNA has increased stability and lower immunogenicity. Further optimization of modRNA led to the discovery of replacing uridine with N1-methyl-pseudouridine to achieve robust translation of modRNA, due to enhancing ribosomal recruitment. Additionally, the use of modRNA-based gene overexpression has been shown to directly program hPSCs to desired cell types, such as hematopoietic progenitors. ModRNA technology has also been used for gene editing. For example, researchers discovered that uridine depletion and chemical modification increased Cas9 mRNA activity and reduce immunogenicity in cell lines and primary CD34+ cells. Scientists also reported that uridine depleted ABE mRNA with 5-methoxyuridine mediates robust editing at various cellular genomic sites, achieving higher efficiency than gene editing using regular unmodified mRNA. The use of modRNA-based CRISPR systems in hPSCs, however, remained unexplored. All said, the use of modRNA to encode and deliver CRSIPR systems carries several advantages over previous methods: 1) it is non-integrating, 2) it does not require transport across the nuclear membrane for expression (as is the case with plasmid delivery), therefore increasing transfection efficiency, 3) it is relatively quick and easy to perform, 4) it requires a minimal starting cell population, and 5) it is only transiently expressed thus greatly reducing the risk of off-target activity.

This study developed modRNA-based genome editing systems for hPSCs that utilize simple lipid-based transfection of sgRNAs, with Cas9 and p53DD or ABE8e modRNA. Using this optimized protocol, 84% KO efficiency was able to achieve in hPSCs.

ModRNA Based Delivery of CIRSPR Components can Successfully Knock Out Genes in hPSCs.

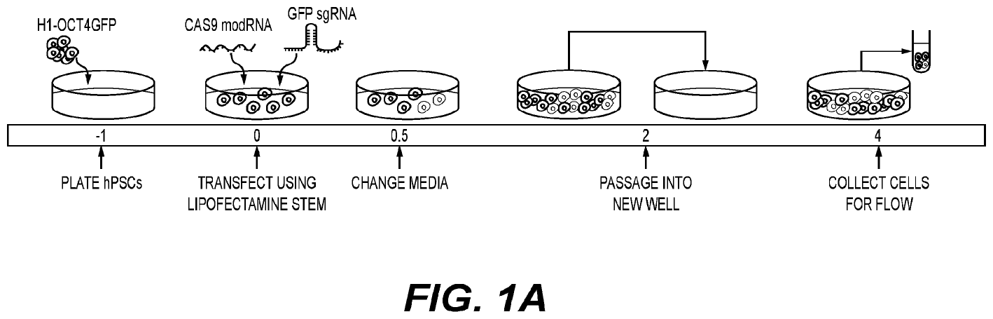

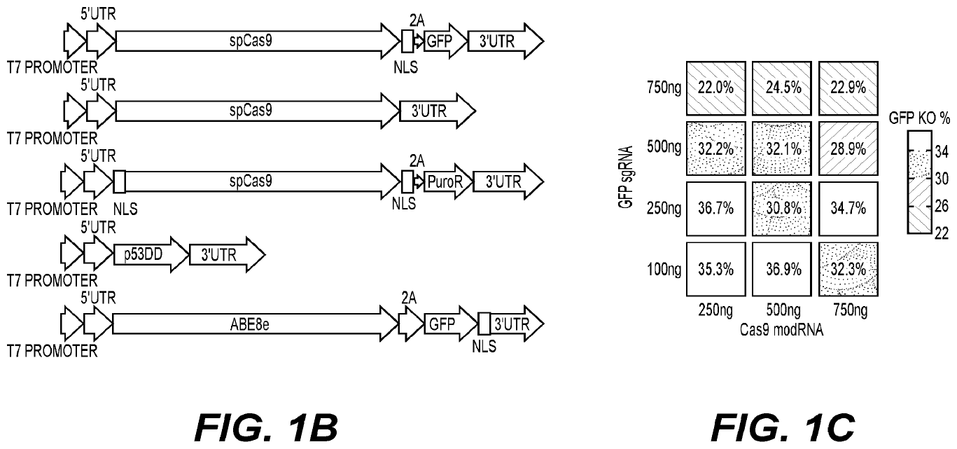

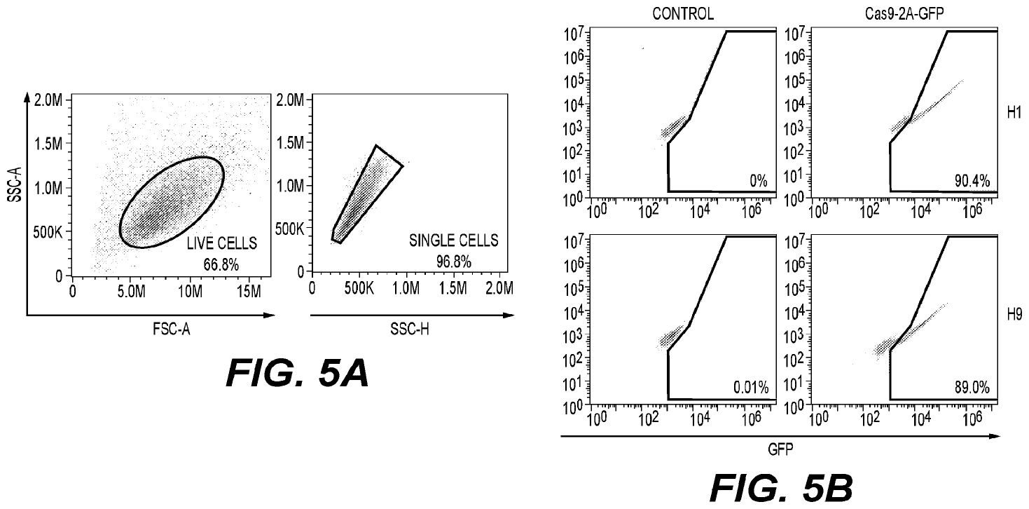

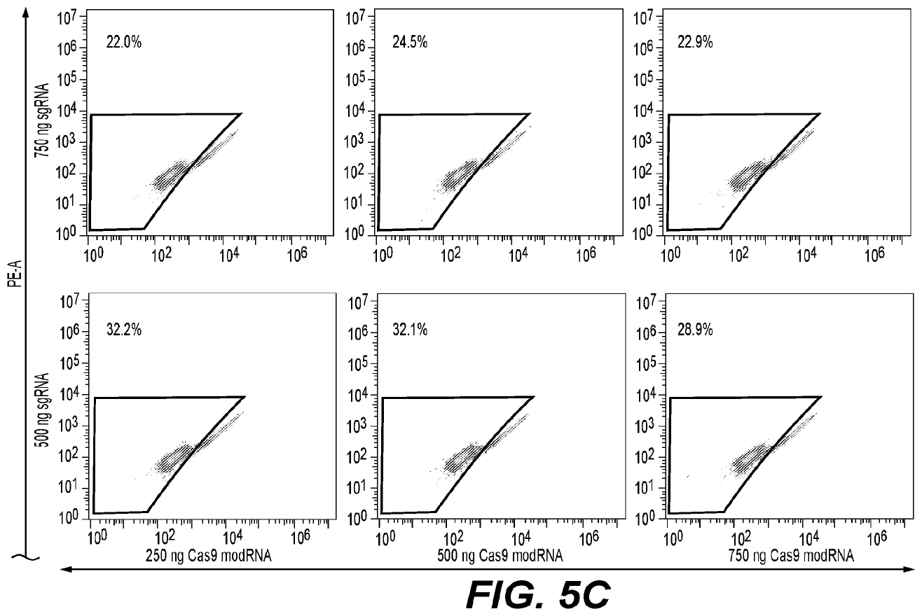

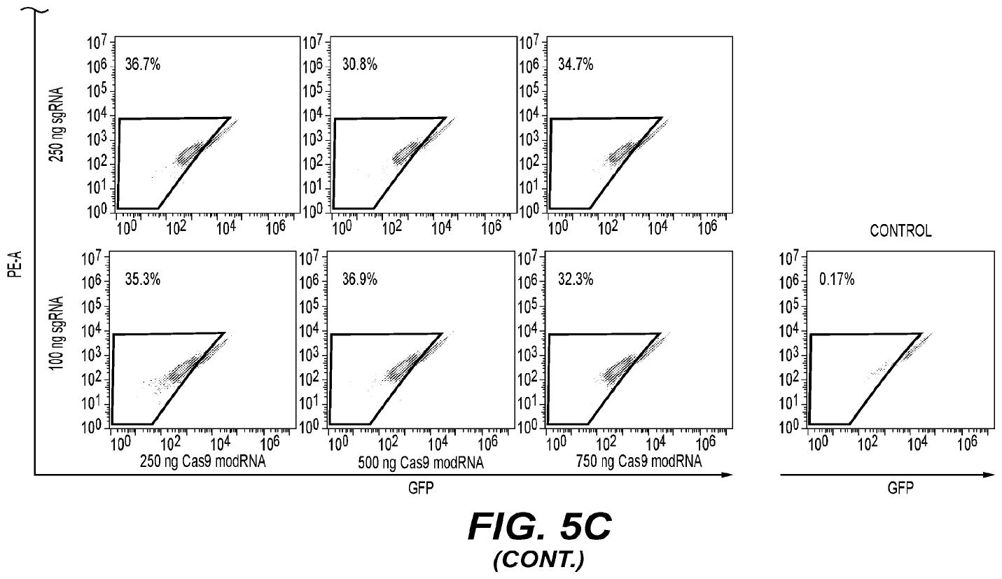

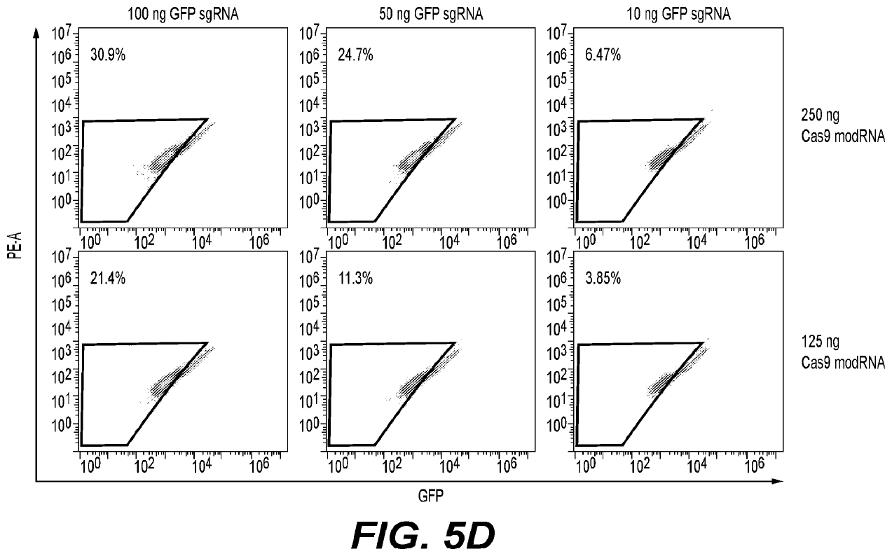

To determine whether Cas9 modRNA can be efficiently delivered to hPSCs using lipofection, Cas9-2A-GFP modRNA containing N1-methyl-pseudo-UTP was synthesized. 2A is a self-cleaving peptide that triggers ribosomal skipping along a single transcript during translation. Incorporation of the 2A linker within the Cas9-2A-GFP modRNA enables the protein synthesis of both Cas9 and GFP from a single modRNA. Cas9-2A-GFP was transfected into H1 and H9 cells. One day later, GFP expression was quantified using flow cytometry. A side scatter height (SSC-H) vs side scatter area (SSC-A) plot was used to exclude doublets for our flow cytometry data analyses (FIG. 5A). Up to 90% transfection efficiency was ale to achieve for Cas9-2A-GFP modRNA based on GFP+ cells (FIG. 5B). Next, to probe for the optimal amount of Cas9 modRNA and target-specific sgRNA, Cas9 modRNA was made without co-expression of GFP to knock out GFP from a human embryonic stem cell (hESC) OCT4-GFP reporter line (H1 OCT4-GFP). For designing the sgRNA targeting GFP, the GFP sgRNA sequence reported by Sanjana et al was used. H1 OCT4-GFP cells were seeded in a 24-well plate and then transfected with different amounts of Cas9 modRNA and GFP sgRNA using Lipofectamine Stem transfection reagent (FIGS. 1A and 1B). Four days after transfection, cells were collected to quantify the percentage of GFP-cells using flow cytometry.

Various amounts of Cas9 modRNA (250, 500, or 750 ng) were tested along with different doses of GFP sgRNA (100, 250, 500, and 750 ng) in H1 OCT4-GFP cells. three Cas9+sgRNA combinations (250+100, 250+250, and 500+100) achieved the highest KO efficiency (˜36% GFP-cells on day 4) (FIGS. 1C and 5C). Fewer amounts of Cas9 modRNA (125, or 250 ng) were also tested along with fewer doses of GFP sgRNA (10, 50, or 100 ng), but it was found that these conditions performed poorly when compared to the three optimal combinations (FIG. 5D). To minimize the total modRNA required for transfection, the 250 ng Cas9 modRNA+100 ng sgRNA combination was used for subsequent experiments.

ModRNA-based CRISPR system efficiently generates gene KOs in hPSCs.

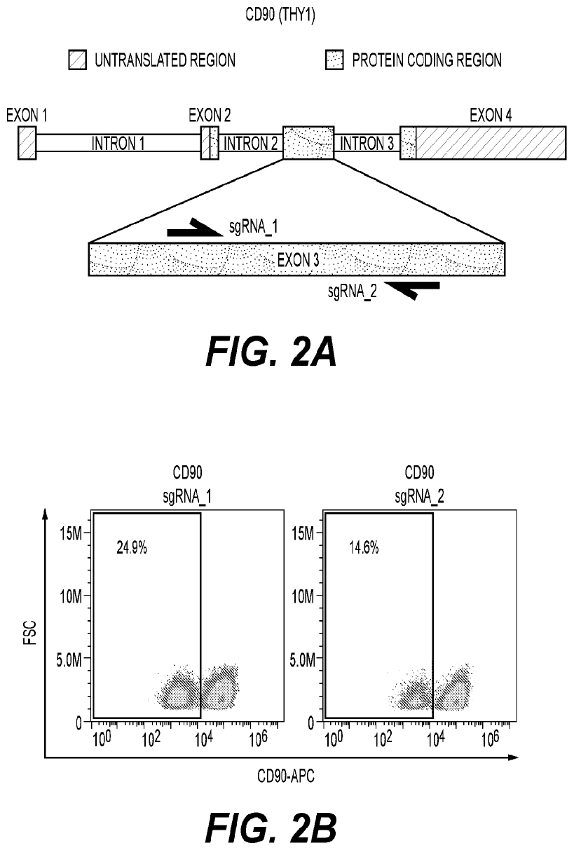

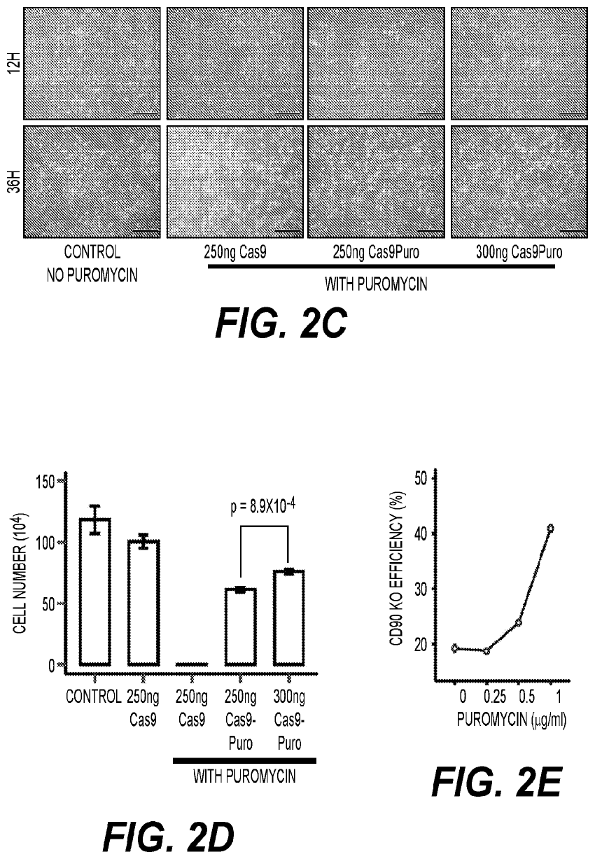

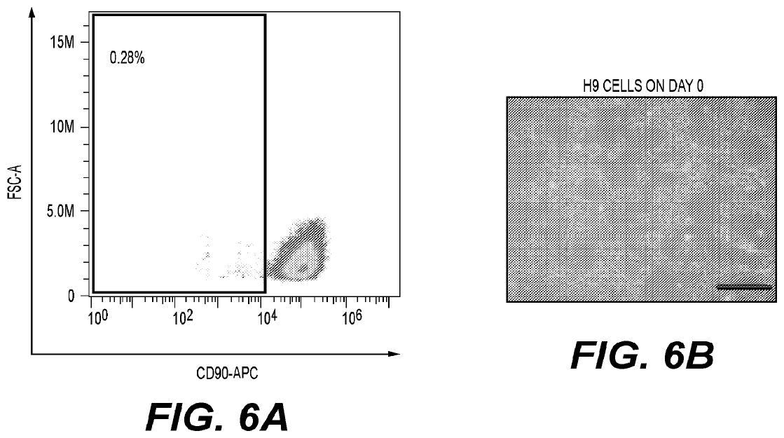

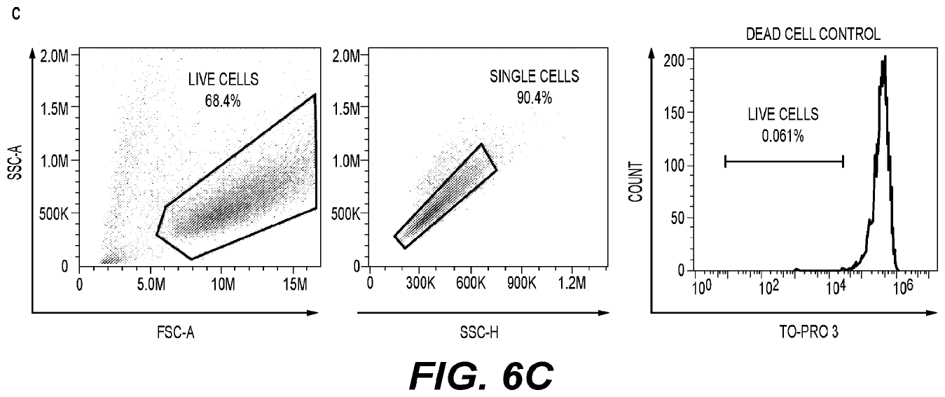

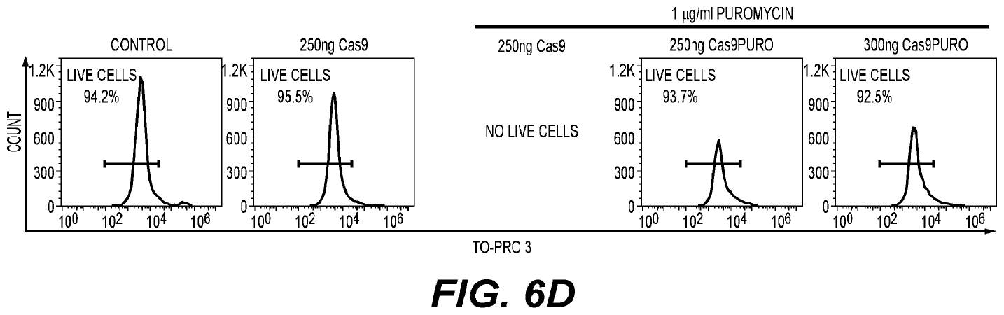

To investigate whether this modRNA-based CRISPR system was able to efficiently knock out genes in hPSCs, THY1 gene that encodes for CD90 protein was targeted, a heavily glycosylated membrane protein that is expressed in undifferentiated hPSCs. Two potential sgRNA target sites for CD90 were selected using ChopChop (FIG. 2A). Seeding the cells too sparsely for endogenous gene KO led to cell detachment and death. To tackle this, initial seeding density was doubled and a Rho-associated kinase (ROCK) inhibitor was included in the culture media which led to better cell survival but reduced transfection efficiency (FIG. 2A). CD90 sgRNA_1 was able to achieve higher KO efficiency than sgRNA_2 and therefore was used for all subsequent experiments (FIGS. 2B and 6A). The next experiment tested if CD90 KO efficiency can be improved via drug selection. Cas9-2A-Puro (Cas9Puro) modRNA was synthesized, which has a puromycin resistance gene linked to the Cas9 via a 2A linker (FIG. 1B). Due to the larger size of the Cas9Puro construct, delivery of 300 ng of Cas9Puro modRNA was also tested in addition to the previously determined 250 ng. H9 cells were seeded onto iMatrix-511 coated wells and transfected with either 250 ng of Cas9, 250 ng of Cas9Puro, or 300 ng of Cas9-Puro modRNA. After 12 hours, cells were treated with 1 μg/ml puromycin. After 24 hours of drug selection, cells were stained with a TO-PRO 3 cell viability dye (excitation/emission 642/661 nm) and counted using a flow cytometer. Treatment with puromycin effectively killed all cells in wells transfected with the Cas9 modRNA. However, in wells that were transfected with the Cas9Puro modRNA, cell survival similar to the untreated control cells was observed indicating that the Cas9Puro modRNA can protect transfected cells from puromycin-mediated cell toxicity (FIGS. 2C-2D, 6B-6D). Additionally, consistently higher cell numbers were observed in wells that were transfected with 300 ng Cas9Puro compared to 250 ng of Cas9Puro, a difference that was statistically significant (p=8.9×10⁴, student's t-test) (FIG. 2D). Due to the higher transfection efficiency using 300 ng of Cas9Puro, as indicated by higher cell survival, 300 ng Cas9Puro modRNA was used for subsequent experiments. To evaluate whether puromycin treatment increases KO efficiency, the Cas9Puro modRNA was used to knock out CD90 in H9 cells accompanied by puromycin treatment at a concentration ranging from 0 to 1 μg/ml. A greater than two-fold increase was oserved in CD90 KO efficiency measured by the percentage of CD90 negative cells on day 5 post-transfection (FIG. 2E).

P53DD greatly increases modRNA-based genome editing efficiency in hPSCs.

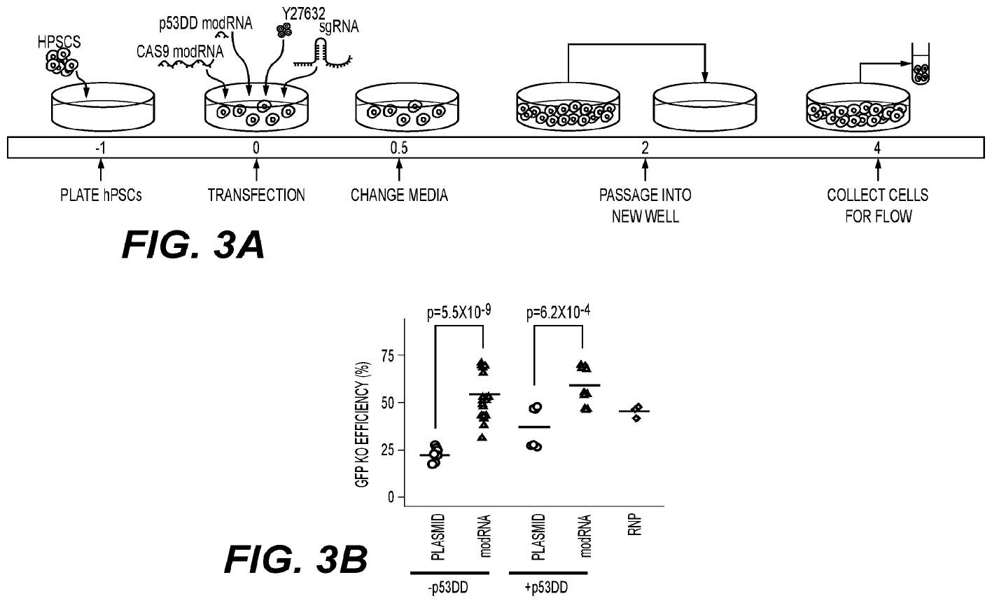

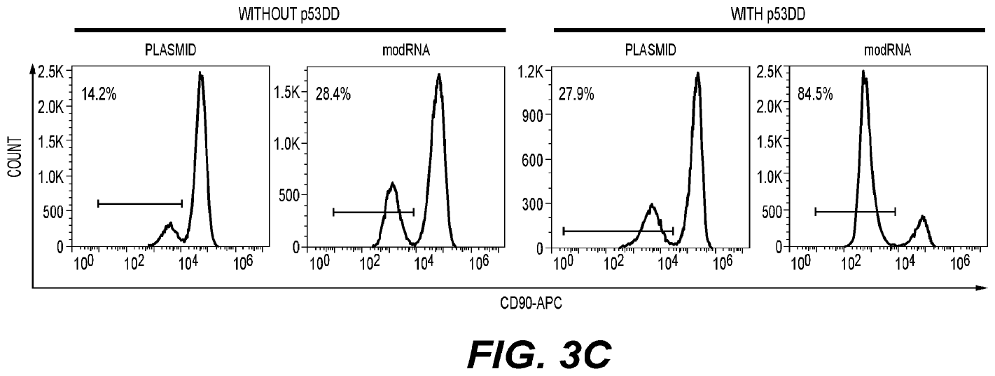

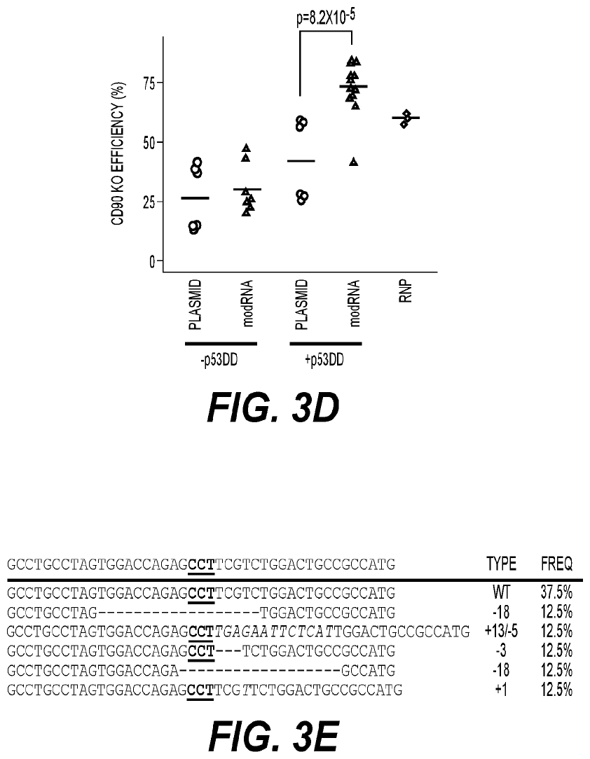

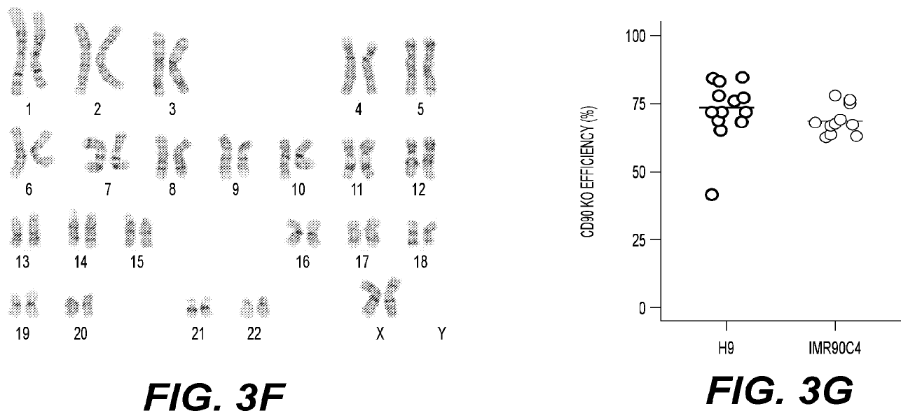

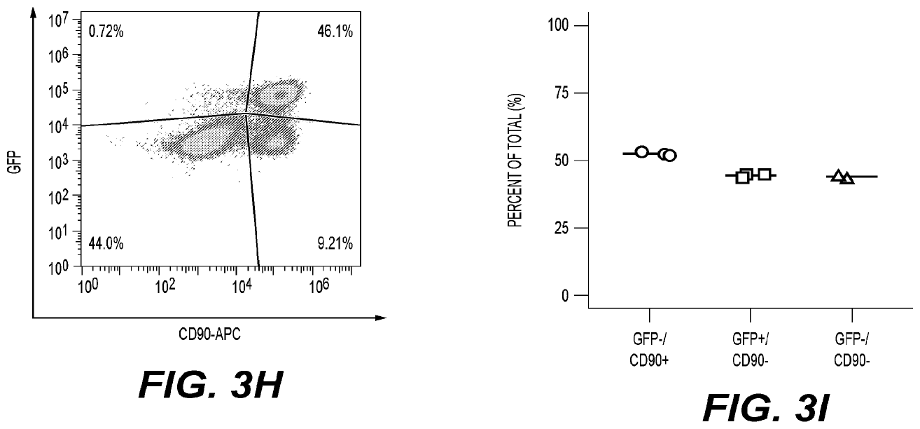

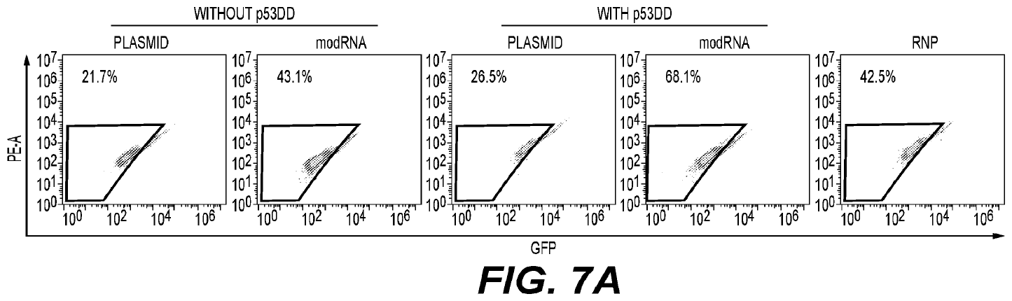

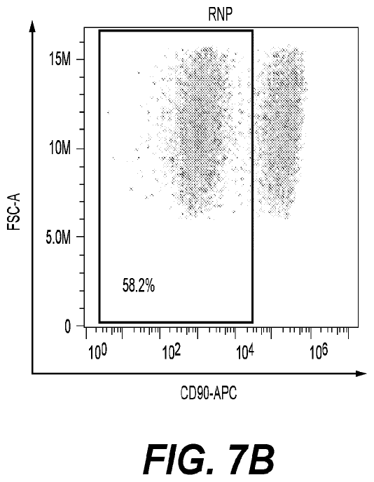

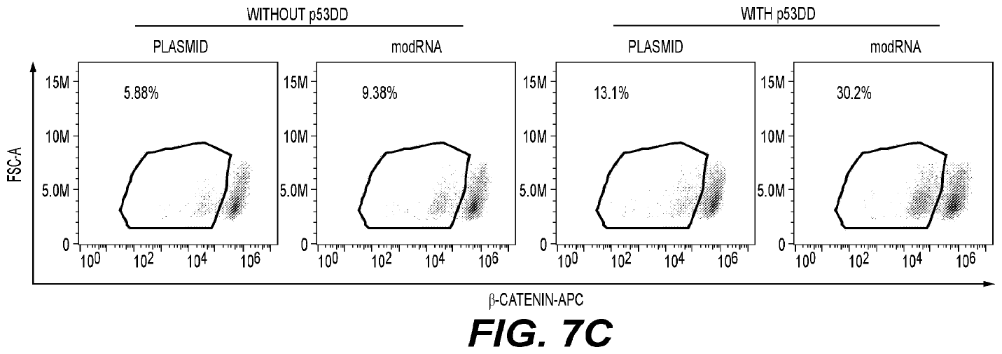

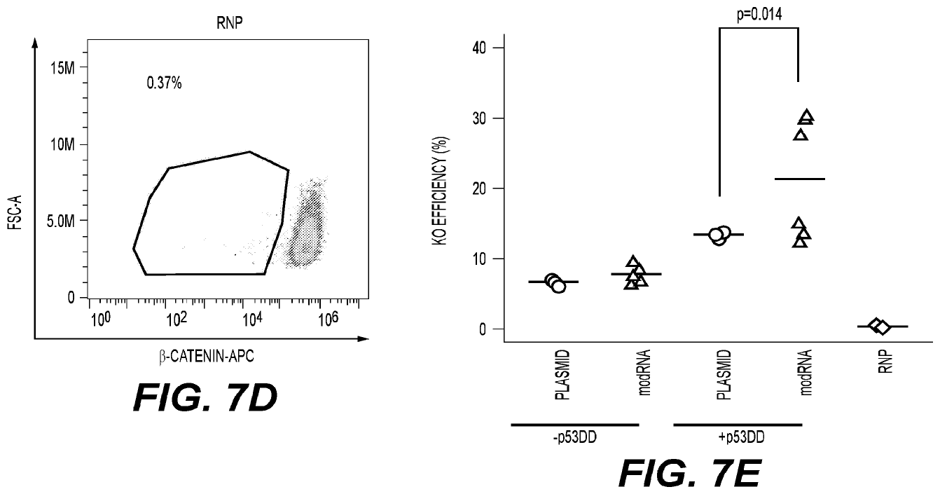

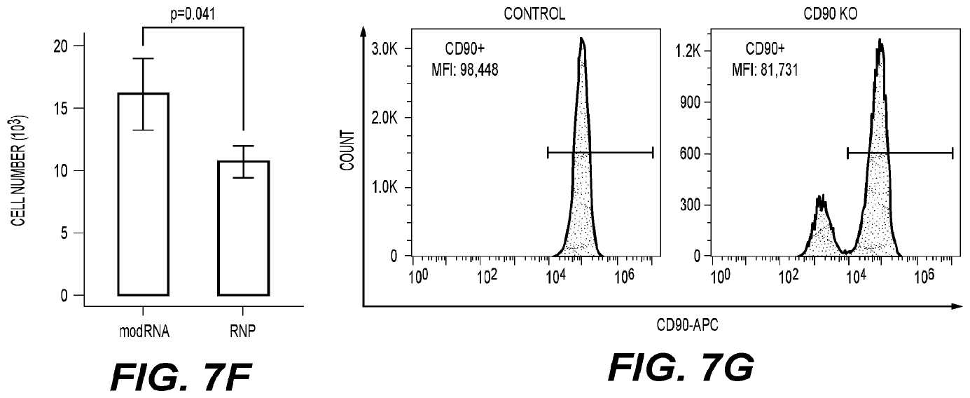

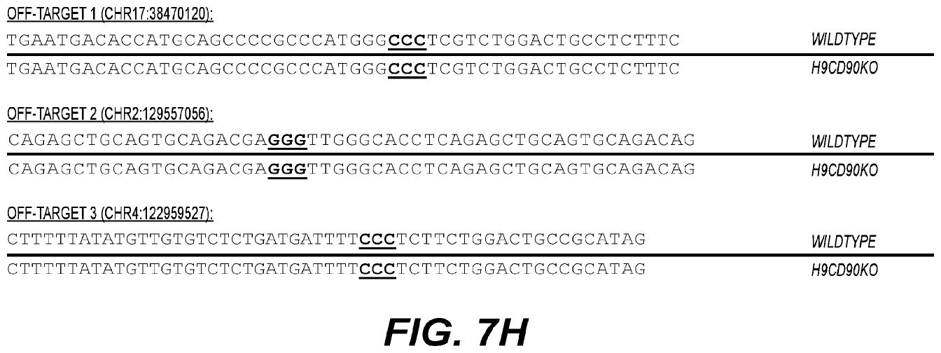

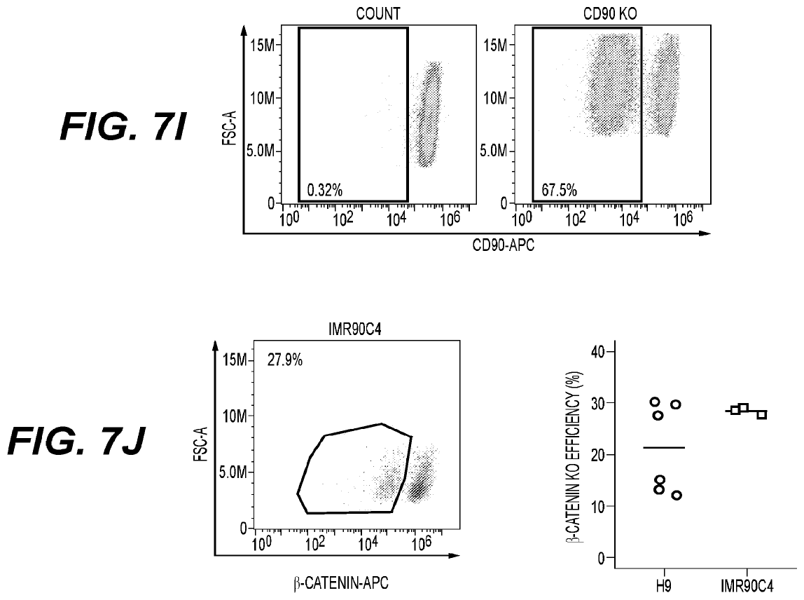

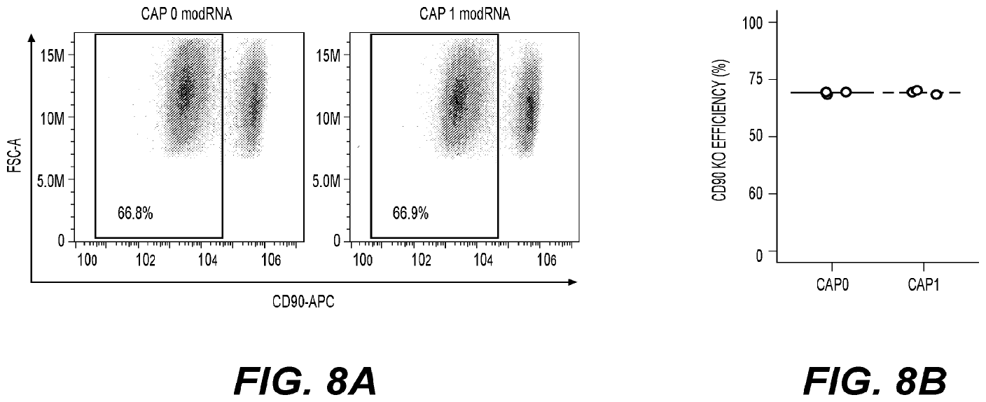

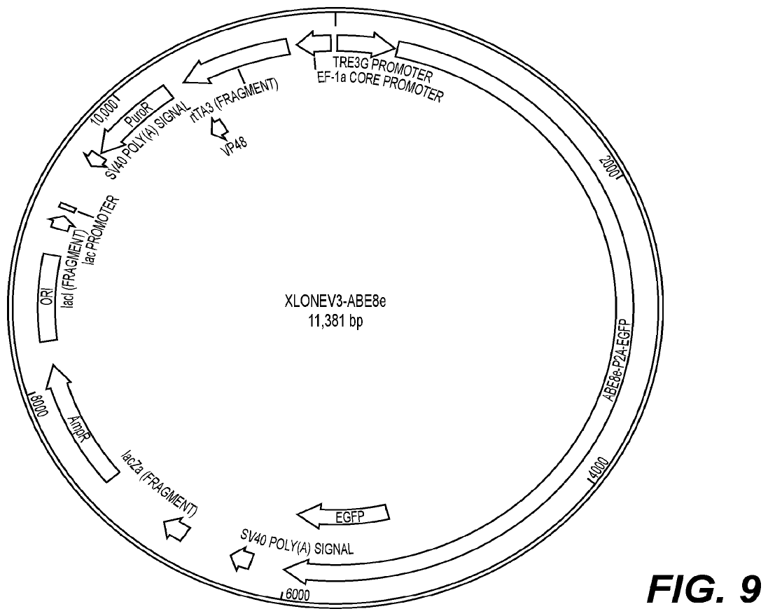

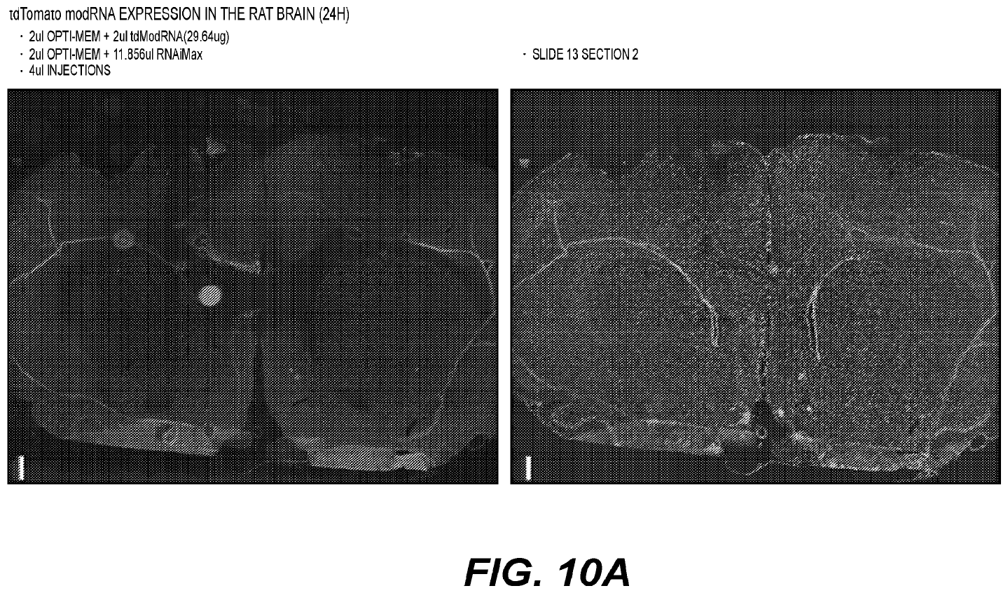

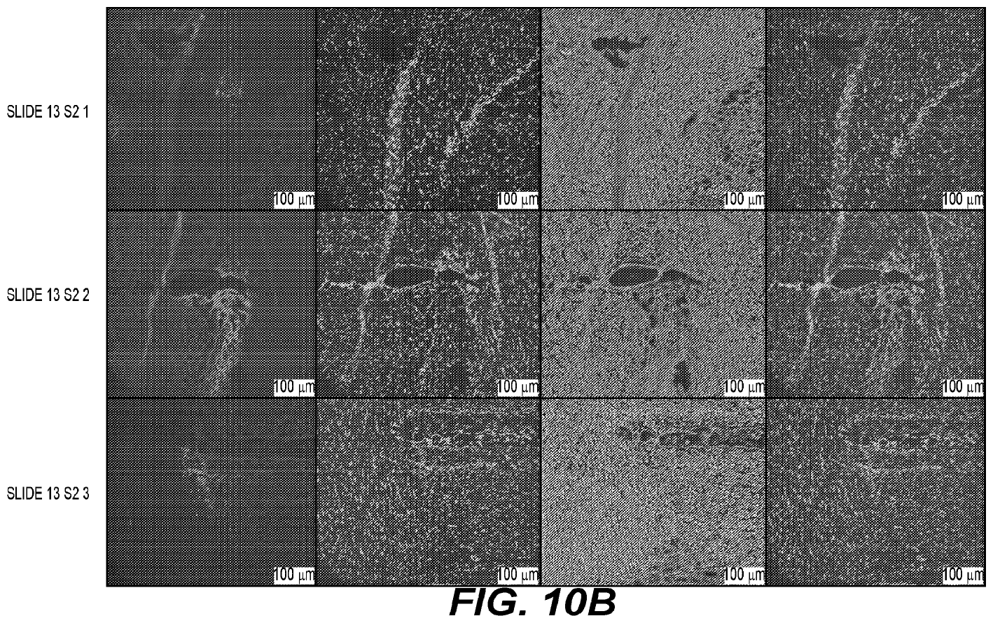

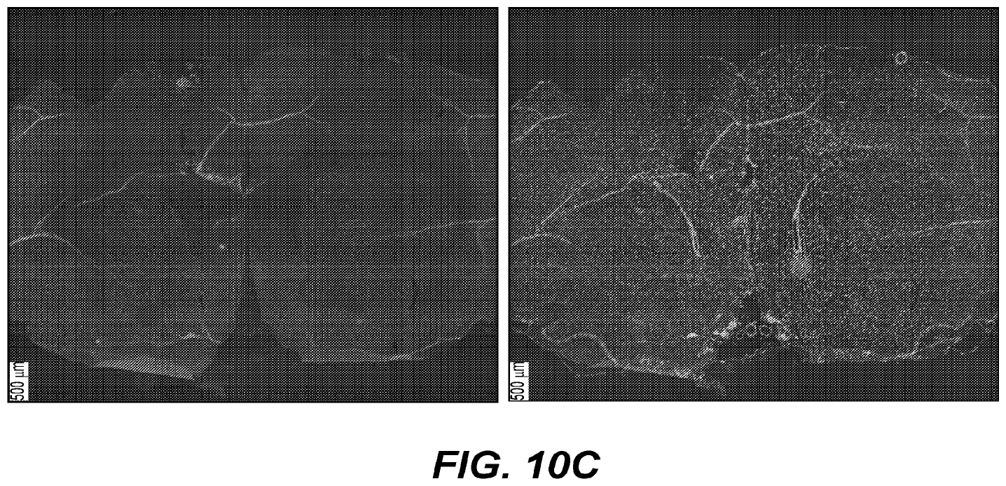

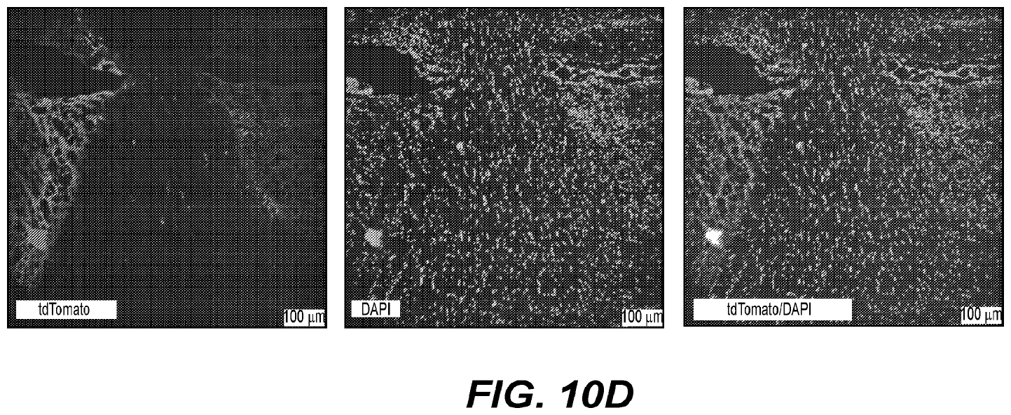

While CRISPR-Cas9 systems have been widely used to engineer genomes of a wide variety of cell types, hPSCs have proven to be exceptionally difficult to engineer due to the toxicity of DSBs in these cells. It was recently reported that the hPSC response to Cas9 induced DSBs is mediated by p53. Additionally, it was shown that p53DD, a dominant negative mutant of p53, can transiently block p53 function and therefore reduce Cas9-induced toxicity in hPSCs. Therefore, p53DD modRNA was synthesized to use with the modRNA-based Cas9 system. To compare modRNA and plasmid-mediated GFP KO in the presence or absence of p53DD, H1 OCT4-GFP cells were transfected with different combination of plasmids or modRNAs (FIG. 3A). For plasmid-based method, hPSCs were transfected with a CRISPR plasmid expressing both Cas9 and sgRNA with or without a p53DD plasmid. For modRNA-based method, hPSCs were transfected with Cas9Puro modRNA, sgRNA, with or without p53DD modRNA. For RNP method, hPSCs were transfected with Cas9 protein coupled with a specific sgRNA. For GFP KO, it was found that modRNA method was superior to plasmid method regardless of p53DD, and modRNA with p53DD yielded the highest KO efficiency among these four conditions, and it was also better than the RNP method (FIGS. 3B, 7A). Next, the study tested CD90 KO in H9 cells with these 5 conditions and it was found that modRNA with p53DD method achieved the highest CD90 KO efficiency, yielding 73.3%±11.2% KO efficiency (FIGS. 3C, 3D, 7B). Moreover, this study tested this modRNA method by knocking out the Wnt signaling effector protein β-catenin. ModRNA with p53DD method achieved the highest β-catenin KO efficiency among five conditions (FIGS. 7C-7E). The RNP method achieved minimal β-catenin KO efficiency using this sgRNA (FIGS. 7C-7E), indicating RNP method can exhibit greater variations in knocking out different genes. Furthermore, RNP method yielded less cells than modRNA method (FIG. 7F).

Next, this study characterized the Cas9 cleavage sites using the TOPO-TA cloning method with CD90 KO cells. A diverse variety of genome editing types were observed in the CD90 KO cells with both insertion and deletion mutations (FIG. 3E). This study also analyzed three potential off-target locations, and no off-target mutations were observed (FIG. 7G). Furthermore, hPSCs edited with the modRNA with p53DD method maintained normal karyotype (FIG. 3F). For modRNA-based gene editing in induced pluripotent stem cells (iPSCs), CD90 KO was compared in H9 cells and IMR90C4 iPSCs, and it was found that the modRNA with p53DD method was equally effective in editing iPSCs, with a KO efficiency of 68.7%+5.1% (FIG. 3G, 7I). Similarly, it was demonstrated that the modRNA with p53DD method generated β-catenin KO at a similar efficiency in iPSCs as in H9 cells (FIG. 7J).

The next experiment examined whether the modRNA-based method can simultaneously target multiple genomic sites and thus knock out multiple gene. We seeded our H1 OCT4-GFP cells and transfected them with Cas9Puro modRNA, GFP sgRNA, CD90 sgRNA, and p53DD modRNA. Cells were collected on day 5 post-transfection to quantify GFP and CD90 expression using flow cytometry. We observed 43.9%±0.3% of cells that were deficient in both GFP and CD90 expression after one single transfection (FIG. 3H, 3I).

Eukaryotic RNA is normally capped at the 5′-end with 7-methylguanosine (m7G), commonly referred to as Cap 0 structure, and is important for translational initiation and prevents degradation of the mRNA transcript. When synthesizing modRNA, the Cap 0 structure is introduced by the addition of the anti-reverse cap analog (ARCA) to the in vitro transcription reaction mix. Higher order eukaryotes will instead have a Cap 1 structure, in which the first nucleotide proximal to the cap structure is methylated. Using modRNA with the Cap 1 modification can abrogate the innate immune response compared to Cap 0 due to its reduced affinity for binding RIG-I, MDA5, and IFIT-1. To synthesize modRNA with the Cap 1 modification, site-directed mutagenesis was used to convert the G to an A proximal to the T7 promotor sequence in the original modRNAc0 plasmid. Then the Cas9Puro insert was cloned into the newly synthesized modRNAc1 plasmid. In addition, the ARCA reagent was replaced with the CleanCap AG reagent. Both cap 0) and cap 1 modRNA can efficiently knock out CD90 in hPSCs (FIG. 7), indicating that the reduced immunogenicity of cap 1 modRNA did not further improve gene KO efficiency in hPSCs.

ABE8e modRNA outperforms its plasmid counterpart for genome editing in hPSCs.

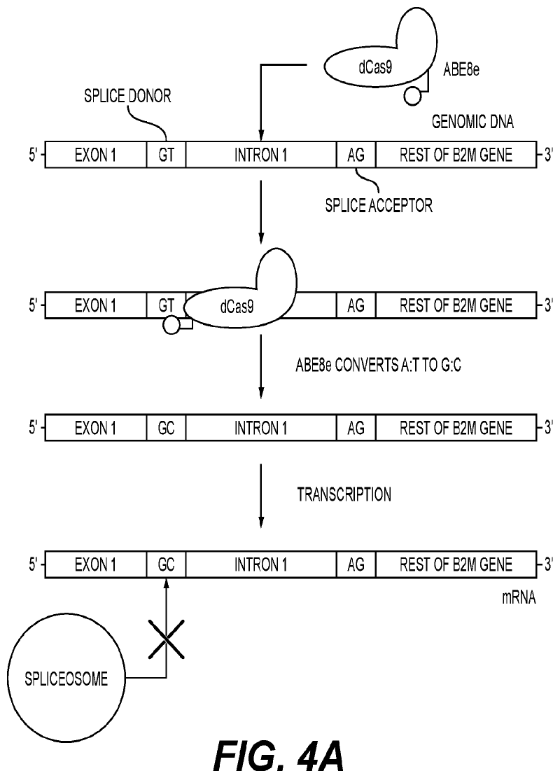

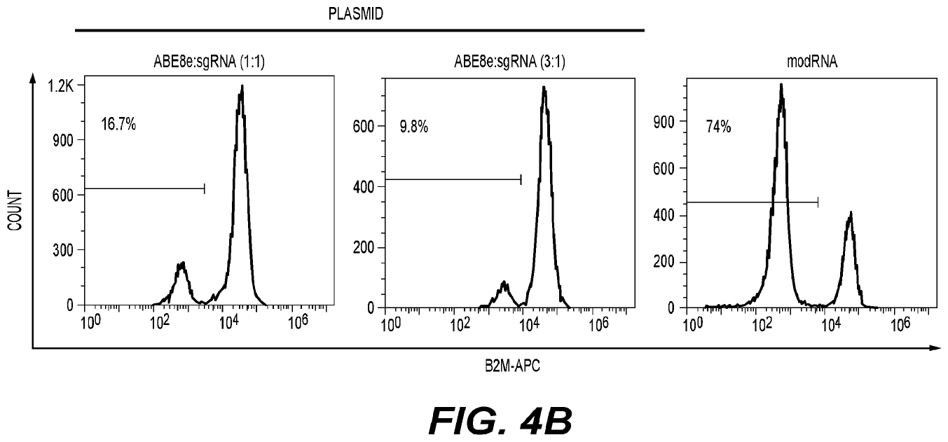

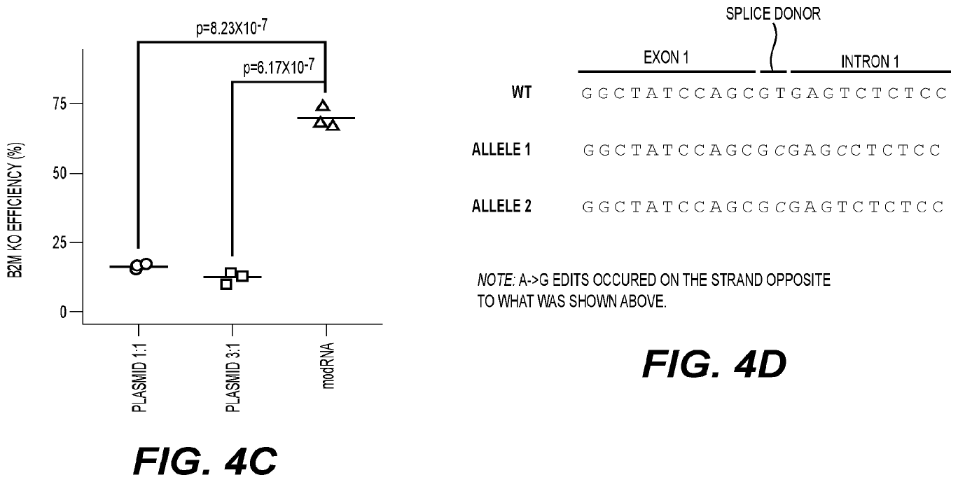

Besides Cas9, base editing can introduce single-nucleotide variants into the genome and represents another important technique for genome editing. The adenosine base editor, ABE8e, was the base editor of choice. To determine if base editing efficiencies using modRNA could outperform plasmid-based delivery, the B2M gene was chosen to knock out, a protein subunit required for surface expression of all class I major histocompatibility complex molecules. This B2M KO strategy employed base editing of the splice donor site, thus, rendering the spliceosome incapable of splicing the transcript correctly and deactivating it (FIG. 4A). Using the SpliceR program, we chose the most efficient sgRNA for B2M KO using the ABE8e system. Next, hPSCs were transfected with ABE8e, which was either encoded by a plasmid or by modRNA, and a sgRNA targeting the splice donor site of intron 1. The plasmid delivery was conducted in two different mass ratios of the ABE8e to sgRNA plasmid (1:1 and 3:1). ABE8e mediated B2M KO efficiencies were then measured using flow cytometry for B2M surface expression 5 days post-transfection. Whereas the plasmid-based method achieved 16.1±0.8% and 12.3±2.2% KO efficiency (1:1 and 3:1 mass ratio respectively), the modRNA-based method generated a much higher KO efficiency (69.6±3.8%) (FIG. 4B, 4C). To ensure that the lack of B2M expression was the result of edited splice donor, intron 1 splice donor site in B2M KO clone was characterized via the TOPO TA cloning method. It was found that both alleles possessed the desired A:T to G:C editing at the splice donor site (FIG. 4D). One allele also had a second base edit, 4 base pairs away within the intron, because this site is still within the ABE8e's base editing window. Overall, these experiments demonstrated that our modRNA-based ABE8e system is about four times more efficient than its plasmid counterpart at generating base edits and enabling gene KO in hPSCs.

This research outlines methods for efficient CRISPR mediated gene Kos in hPSCs using a modRNA-based Cas9 or ABE8e system, which can be widely adopted for most labs without requiring electroporation or nucleofection devices. The efficacy of the modRNA Cas9 system was tested using multiple hPSC lines, including two hESC lines as well as a human iPSC line, demonstrating the general applicability. This approach is highly flexible to a variety of experimental conditions owing to the Cas9Puro modRNA which can be used in conjunction with puromycin treatment to increase KO efficiency when high transfection efficiency is not possible for certain cell types. Integration of the p53DD modRNA into the system significantly increases gene KO efficiency by reducing Cas9 induced DSB toxicity in hPSCs.

This study also assessed B2M KO in hPSCs via inactivation of the splice donor using the ABE8e base editor. It was found that modRNA ABE8e method is more efficient compared to the plasmid format. The main advantage of using base editors for generating gene KO in hPSCs is the elimination of DSBs generated by Cas9. This abolishes the undesired chromosomal rearrangements that result from DSBs and lowers the chances of detrimental off-target insertions or deletions, thus providing a more clinically relevant genome engineering tool for hPSCs. This modRNA ABE8e method had a KO efficiency similar to that of our Cas9 with p53DD modRNA method (69.6±3.8% vs. 73.3%±11.2%), highlighting its potential as an alternative to CRISPR-Cas9 based strategies.

In summary, this study demonstrated that when CRISPR-Cas9 with p53DD or ABE8e modRNA is transfected into hPSCs, it outperforms the plasmid-based method. The increased efficiency of modRNA methods is likely due to higher transfection efficiency and higher Cas9 or ABE8e protein expression level in hPSCs for modRNA method. Since it is not 100% efficient though, as is the case with other delivery methods, clonal isolation is still required for some downstream gene KO studies. Despite this, these Cas9 with p53DD or ABE8e modRNA methods result in extremely higher transfection efficiency and very high Cas9 or ABE8e expression levels, ultimately generating higher KO efficiencies in hPSCs.

### Example 2. Methods and Materials

TABLE 1
Key resources table.

| | | |
|---|---|---|
| REAGENT or RESOURCE | SOURCE | IDENTIFIER |

Antibodies

| | | |
|---|---|---|
| B2M-APC | Biolegend | 316312 |
| CD90-APC | Biolegend | 328113 |
| β-Catenin | BD | 610153 |
| Goat anti-mouse IgG, | Thermo Fisher | A-21235 |
| Alexa Fluor 647 | Scientific | |

Recombinant DNA

| | | |
|---|---|---|
| PB-CRISPR | Addgene | 160047 |
| pCE-mp53DD | Addgene | 41856 |
| pGuide (for DNA plasmid | Addgene | 64711 |
| gRNA delivery) | | |
| XLone V3-ABE8e | This paper | Pending |
| | | submission |
| modRNAc0-Cas9 | This paper | Addgene 170180 |
| modRNAc0-Cas9-2A-GFP | This paper | |
| modRNAc0-Cas9-2A-Puro | This paper | Addgene 172855 |
| modRNAc0-p53DD | This paper | Addgene 176902 |
| modRNAc0-ABE8e | This paper | Addgene 178177 |

Chemicals, peptides, and recombinant proteins

| | | |
|---|---|---|
| iMatrix-511 | Iwai North America Inc | N-892021 |
| mTeSR1 | StemCell Technologies | 85850 |
| Accutase | Innovative Cell | AT104500 |
| | Technologies | |
| DMEM | Thermo Fisher | 11965118 |
| | Scientific | |
| Y-27632 | Selleck Chemicals | 146986-50-7 |
| N1-methyl-pseudo-UTP | TriLink | N-1081 |
| | Biotechnologies | |
| Anti-Reverse Cap Analog | TriLink | N-7003 |
| (ARCA) | Biotechnologies | |
| CleanCap AG | TriLink | N-7113 |
| | Biotechnologies | |
| Lipofectamine Stem | Thermo Fisher | STEM00015 |
| Transfection Reagent | Scientific | |
| Opti-MEM Reduced | Thermo Fisher | 51985034 |
| Serum Medium | Scientific | |
| Doxycycline | Sigma-Aldrich | D9891 |
| PBS | Thermo Fisher | 14190250 |
| | Scientific | |
| Bovine Serum Albumin | VWR | 10842-692 |
| GoTaq G2 Hot Start | Promega | M7422 |
| Master Mix | | |
| LB Broth medium | Thermo Fisher | 10855001 |
| | Scientific | |
| Puromycin | Thermo Fisher | A1113803 |
| | Scientific | |

Critical commercial assays

| | | |
|---|---|---|
| ZymoClean Gel DNA | Zymo Research | D4001 |
| Recovery kit | | |
| In-Fusion HD Cloning | Takara Bio | 638916 |
| Plus CE kit | | |
| DNA Clean & | Zymo Research | D4004 |
| Concentrator-5 | | |
| MEGAscript T7 | Thermo Fisher | AM1334 |
| Transcription kit | Scientific | |
| MEGAclear transcription | Thermo Fisher | AM1908 |
| clean-up kit | Scientific | |
| EnGen sgRNA Synthesis | NEB | E3322 |
| kit, S. pyogenes | | |
| Zymo Quick DNA | Zymo Research | D4068 |
| Miniprep Plus kit | | |
| TOPO TA Cloning Kit | Thermo Fisher | 45-003-0 |
| for Sequencing | Scientific | |
| Zyppy Plasmid Miniprep | Zymo Research | D4020 |
| Kit | | |

Experimental models: Cell lines

| | | |
|---|---|---|
| Human: H9 hESCs | WiCell | WB0299 |
| Human: H1 OCT4-GFP | WiCell | MCB-01 |
| hESCs | | |
| Human: IMR90C4 iPSCs | WiCell | WB65317 |

Software and algorithms

| | | |
|---|---|---|
| FlowJo | www.flowjo.com/ | N/A |

Oligonucleotides

| | | |
|---|---|---|
| For gene cloning, | This paper | See Table 2 |
| sequencing editing sites, | | |
| and gRNA sequences. | | |

TABLE 2
Oligonucleotides used in this paper for gene cloning, sequencing editing sites, and
gRNA sequences.
Gene Cloning into modRNAco vector

| | |
|---|---|
| Cas9 | Forward: CATGGCATGCGAATTCATGGACAAGAAGTACTCCATTGGGC (SEQ ID |
| | NO: 33) |
| | Reverse: AAGCGAGCTCACTAGTTTAGTCTCCACCGAGCTGAGAG (SEQ ID NO: |
| | 34) |
| Cas9-2A-Puro | Forward: CATGGCATGCGAATTCGCCACCATGGATTACAAAGACG (SEQ ID NO: |
| | 35) |
| | Reverse: AAGCGAGCTCACTAGTTCAGGCACCGGGCTTGCG (SEQ ID NO: 36) |
| Cas9-2A-GFP | Forward: CATGGCATGCGAATTCATGGACAAGAAGTACTCCATTGGGC (SEQ ID |
| | NO: 33) |
| | Reverse: AAGCGAGCTCACTAGTTTACTTGTACAGCTCGTCCATGCC (SEQ ID |
| | NO: 37) |
| p53DD | Forward: CATGGCATGCGAATTCGCCACCATGACTGCCATGG (SEQ ID NO: 38) |
| | Reverse: AAGCGAGCTCACTAGTTCAGTCTGAGTCAGGCCCC (SEQ ID NO: 39) |
| ABE8e-GFP | Forward: CATGGCATGCGAATTCGCCACCATGAAACGGACAGC (SEQ ID NO: |
| | 40) |
| | Reverse: AAGCGAGCTCACTAGTTTATACCTTACGCTTCTTCTTTGGC (SEQ ID |
| | NO: 41) |

Sequencing primers for editing sites

| | |
|---|---|
| CD90 On-target | Forward: ATCTCTCCACTTCAGGTGGGT (SEQ ID NO: 42) |
| | Reverse: TGTATTTGCTGGTGAAGTTGGT (SEQ ID NO: 43) |
| CD90 Off-target | Forward: AGAGAGGGTGTCAGGGAGGT (SEQ ID NO: 44) |
| 1 | Reverse: CTAAAAAGCCGCGAAGACAG (SEQ ID NO: 45) |
| CD90 Off-target | Forward: CTCACAGGCATTCACAAGGA (SEQ ID NO: 46) |
| 2 | Reverse: GCAGGAGTCACTGTCTGCAC (SEQ ID NO: 47) |
| CD90 Off-target | Forward: TTGTGGACCTGCATGTTTGT (SEQ ID NO: 48) |
| 3 | Reverse: CACAAACACTACAGAGGTTTTGTATTC (SEQ ID NO: 49) |
| B2M splice | Forward: GCGTTTAATATAAGTGGAGGCG (SEQ ID NO: 50) |
| donor site | Reverse: CACCAAGGAGAACTTGGAGAAG (SEQ ID NO: 51) |

gRNA sequences

| | |
|---|---|
| GFP sgRNA | GGGCGAGGAGCTGTTCACCG (SEQ ID NO: 52) |
| CD90 sgRNA-1 | CATGGCGGCAGTCCAGACGA (SEQ ID NO: 53) |
| CD90 sgRNA-2 | GCCTTCACTAGCAAGGACGA (SEQ ID NO: 54) |
| B2M sgRNA | ACTCACGCTGGATAGCCTCC (SEQ ID NO: 55) |
| b-catenin | GAAACAGCTCGTTGTACCGC (SEQ ID NO: 56) |
| sgRNA | |

Cell lines. Three pluripotent cell lines, H9, H1 OCT4-GFP, and IMR90C4, were used for this study. These lines were obtained from WiCell Research Institute. All cell culture experiments involving human pluripotent stem cell lines were approved by the Embryonic Stem Cell Oversight Committee at the Pennsylvania State University and carried out in accordance with the approved guidelines.

Maintenance of hPSCs. hPSCs were maintained on iMatrix-511 (Iwai North America) coated plates in mTeSR1 medium (STEMCELL Technologies). Cells were regularly passaged when they reached 80-90% confluency, usually 3-4 days after the previous passage. For passaging, cell medium was aspirated and 1 ml of Accutase (Innovative Cell Technologies) was added to each well. Cells were incubated at 37° C., 5% CO₂ for 5 to 10 minutes. Dissociated cells were transferred to excess DMEM at a 1:2 (vol/vol) ratio and centrifuged at 1000 rpm for 4 minutes. New wells were precoated with 0.75 μg/ml iMatrix-511 and incubated at 37° C., 5% CO₂ for 10 minutes. After centrifugation, cell pellet was resuspended in mTeSR1 with 5 μM Y-27632 (Selleck Chemicals). 10,000-20,000 cells/cm² were seeded onto iMatrix-511 coated wells. For regular maintenance cells were cultured in six-well plates.

Modified mRNA (modRNA) synthesis. Cas9-2A-GFP, Cas9, Cas9Puro, p53DD, and ABE8e template DNA was PCR amplified from the donor plasmid using appropriate primers. The PCR product was run on a 1% Agarose gel and the band at the appropriate size was excised and the DNA extracted using the Zymoclean Gel DNA Recovery kit (Zymo Research). Purified insert DNA was cloned into the linearized modRNACO plasmid using the In-Fusion Cloning Kit (Takara Bio). The DNA template for modRNA synthesis was PCR amplified from the successfully cloned modRNAc0 plasmid followed by PCR purification using DNA Clean & Concentrator-5 (Zymo Research). ModRNA was synthesized from the PCR DNA template via in vitro transcription (IVT) using the MEGAscript T7 Transcription kit (ThermoFisher) supplemented with 8.1 mM ATP, 2.7 mM GTP, 8.1 mM CTP, 2.7 mM N1-methyl-pseudo-UTP (TriLink Biotechnologies), and 10 mM Anti-Reverse Cap Analog (ARCA) (TriLink Biotechnologies). The IVT reaction product was treated with DNase I to remove DNA template and then purified using the MEGAclear transcription clean-up kit (ThermoFisher). RNA concentration was measured using a NanoDrop (ThermoFisher).

sgRNA synthesis. sgRNA was synthesized using the EnGen sgRNA Synthesis kit (NEB). Target specific oligos were ordered from Integrated DNA Technologies using the following template:

(SEQ ID NO: 28)
TTCTAATACGACTCACTATAG(N)₂₀GTTTTAGAGCTAGA

Gene-specific target sequences for CD90 were selected using the ChopChop online tool. The IVT reaction was assembled based on the manufacturer's recommendations and the sgRNA was purified using an RNA Clean & Concentrator-5 kit (Zymo Research). RNA concentration was measured using a NanoDrop (ThermoFisher).

Transfection of Cas9 modRNA or plasmid into hPSCs. For Cas9 mediated gene KO, ˜13,000 cells/cm² hPSCs were seeded onto iMatrix-511 coated wells of a 24-well plate and cultured for 24 hours at 37° C., 5% CO₂. The transfection mix was prepared using either modRNA or plasmid Cas9/Cas9Puro, target specific sgRNA, p53DD, and Lipofectamine Stem Transfection Reagent (ThermoFisher) (1:2 ratio, mass/volume) in Opti-MEM medium (ThermoFisher). Before transfection, the spent medium was replaced with fresh mTeSR1 with 10 μM Y-27632. The transfection mix was incubated at room temperature for 10 minutes and then added to the well in a dropwise fashion followed by a media change 12 hours later. From then on, cells were maintained in mTeSR1 with daily media changes until cells were eventually collected for flow cytometry. Transfection of ABE8e modRNA or plasmid into hPSCs. For ABE8e mediated gene KO, H9 cells were seeded onto iMatrix-511 coated wells of a 12-well plate and cultured at 37° C., 5% CO₂. Upon reaching 30% confluency, fresh 0.5 ml mTeSR1 was added to each well, and the cells were transfected using Lipofectamine Stem Transfection Reagent (ThermoFisher) in Opti-MEM medium (ThermoFisher). For plasmid-based method, cells were transfected using 500 ng (1:1) or 750 ng (3:1) XloneV3-ABE8e plasmid (which results in Doxycycline induced expression of ABE8e), 500 ng (1:1) or 250 ng (3:1) pGuide_B2M_Exon1 plasmid, and 5 mg/ml Doxycycline (Sigma-Aldrich). For modRNA-based method, cells were transfected using 600 ng ABE8e modRNA and 200 ng B2M_Exon1_sgRNA. 24 hours post transfection, a complete media change was performed using fresh mTeSR1 media, with 5 mg/mL Doxycycline supplemented to the plasmid transfected wells. Cells were cultured further for another 4 days, with daily mTeSR1 media changes, and with 5 mg/mL Doxycycline for the plasmids treated cells. 5 days post-transfection, samples were analyzed for B2M expression using flow cytometry.

Flow cytometry. hPSCs were dissociated into single cells with 1 ml Accutase for 10 to 15 minutes. Cells were then resuspended in FlowBuffer-1 (DPBS with 0.5% BSA) and immunostained with appropriate conjugated primary antibodies. Data was collected on a BD Accuri C6 Plus flow cytometer and processed using the Flowjo software.

TOPO TA cloning for sequencing. hPSCs were cultured in a well of a 6-well plate until reaching 80% confluency. Once reaching this confluency, genomic DNA was then isolated using the ZYMO Quick DNA Miniprep Plus kit (Zymo Research). This genomic DNA was then used as a template for PCR amplification of genomic regions of interest. PCR was carried out using GoTaq DNA polymerase (Promega) with appropriate primers. The resulting amplicons were run through 1% agarose gels, and bands of interest were gel purified using the Zymoclean Gel DNA Recovery kit (Zymo Research) and subsequently run through the Zymo clean and concentrator-5 kit (Zymo Research). The resulting amplicons were then cloned into the TOPO TA cloning plasmid using the TOPO TA Cloning Kit for Sequencing (Thermofisher) according to the manufacturer's instructions. The resulting cloned plasmids were finally transformed into One Shot Stb13 E. coli cells (Thermofisher) according to manufacturer's instructions, plated on Ampicillin agar plates, and cultured at 37° C. overnight. Single E. coli colonies were then picked and cultured in LB broth overnight, cultured at 37° C. and shaking at 250 rpm. The next day, plasmids were purified using the Zyppy Plasmid Miniprep Kit (Zymo Research) and sent in for sequencing.

Quantification and statistical analysis. Quantification of flow cytometry data is shown as mean±S.D. unless otherwise stated. One-way ANOVA followed by a post-hoc Tukey's Test was used for comparison between multiple groups. Unpaired student's t-test was used for comparison between different experimental groups. P values≥0.05 were considered not significant; p<0.05 was considered significant.

### Example 3. Cell-Type-Specific modRNA Expression Via miRNAs and CRISPR-Csy4

Advances in chemically modified mRNA (modRNA) research in the past two decades, especially the major success of modRNA vaccines against SARS-CoV-2, have brought increased attention to RNA-based therapies. Although modRNA is a safe, transient, and non-immunogenic delivery platform that rapidly translates genes, this technology could not achieve cell-type-specific resolution. How to design novel modRNA constructs which enables translation of modRNA exclusively in one cell type but not others is a fundamental question which is critical for both basic science research and therapeutic applications. For example, myocarditis, one of COVID modRNA vaccine side effects, is due to the undesired expression of modRNA vaccine in heart muscle cells. If we can design new modRNA vaccine which allows modRNA vaccine expression only in skeletal muscle cells, we can eliminate myocarditis and many other side effects.

To achieve this goal, this study designed a regulatory modRNA (modRNA-1) including Csy4 and miRNA binding sites, and a gene of interest (GoI) modRNA (modRNA-2) containing gene of interest (e.g. COVID S-gene) and a 28-nucleotide Csy4 recognition sequence. With these two modRNAs, we can achieve cell type specific expression of GoI.

In order to restrict COVID S-gene expression only in skeletal muscle cells, miR-206 binding sites were incorporated in modRNA-1. miR-206 is only expressed in skeletal muscle cells, not cardiac muscle cells. Therefore, miR-206 in skeletal muscle cells will bind to modRNA-1 because it has miR-206 binding sites and then degrades modRNA-1 and eliminate Csy4 expression. Thus, S-gene in modRNA-2 will be able to express in skeletal muscle cells.

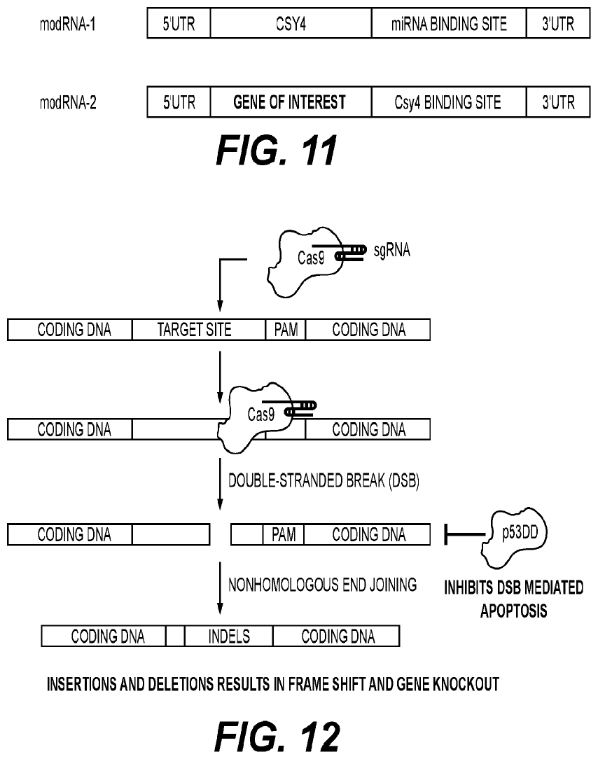

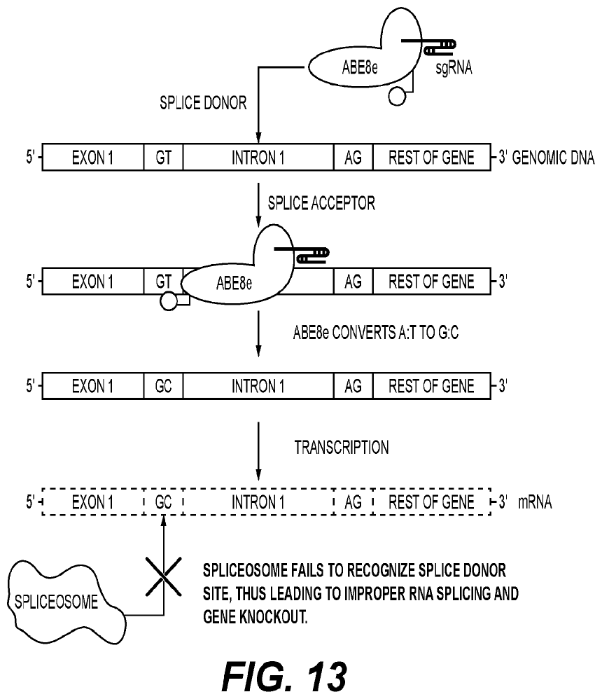

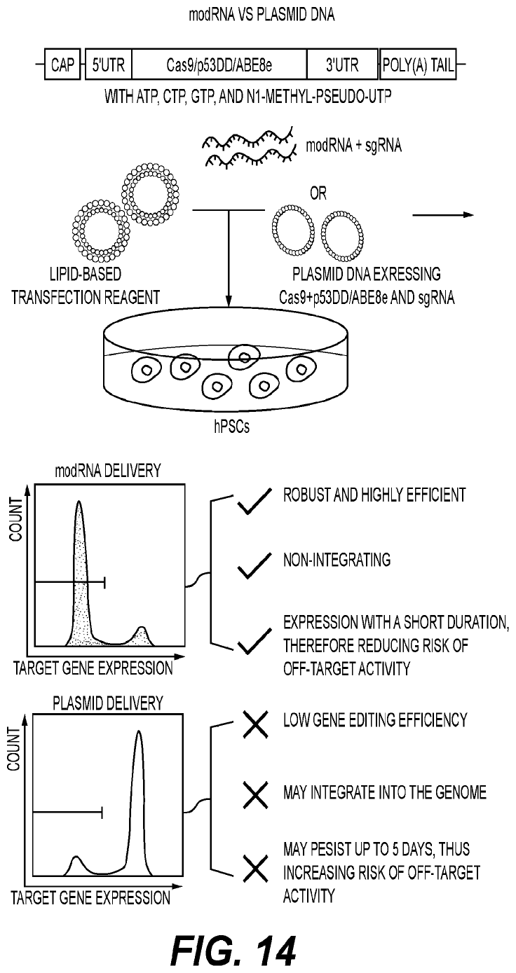

In cardiac muscle cells lacking miR-206, modRNA-1 can survive and Csy4 can be expressed. Then Csy4 can eliminate modRNA-2. Therefore, S-gene is not expressed in cardiac muscle cells (FIG. 11).

Overall, with miR-206 incorporation in modRNA-1, only skeletal muscle cells expressed S-gene.

This cell-type-specific modRNA expression system is a platform technology. This can be used for achieving expression in many different type cells by changing miRNA binding site in modRNA-1. miR-218 or miR-375 binding site can be used to restrict expression only in motor neuron, or pancreatic islet cells respectively.

### Example 4. ModRNA-Based Base Editor (ABE8e) Technology

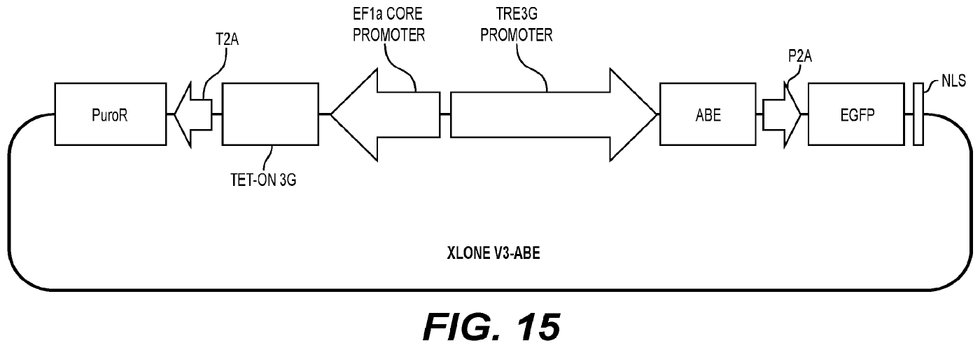

XLOneV3-ABE8e cloning. Backbone vector was linearized from XLOne-Puro NFIL3 P2A-eGFP NLS plasmid (Addgene #140026) with Kpn I and Nhe I-HF digestion for 1 hour. The 6.5 kb band was excised and recovered from gel. ecTadA (8e V106W)-nSpCas9 amplicon was amplified from plasmid ABE8e (TadA-8e V106W) (Addgene #138495) with Q5 polymerase (NEB #M0491S). The plasmid (FIG. 15) was ligated with In-Fusion ligase (Takara Bio) and transformed into Stb13 Chemically Competent E. coli (Thermo Fisher).

ModRNA of ABE8e. ABE8e-GFP was amplified and cloned into modRNA 5MCS3 plasmid. ABE8e-GFP modRNA was synthesized via in-vitro transcription using the MEGAscript T7 Transcription Kit with N1-methylpsuedouridine and ARCA (Anti-Reverse Cap Analog). The IVT reaction product was treated with Dnase I and purified with the MEGAclear Transcription Cleanup Kit.

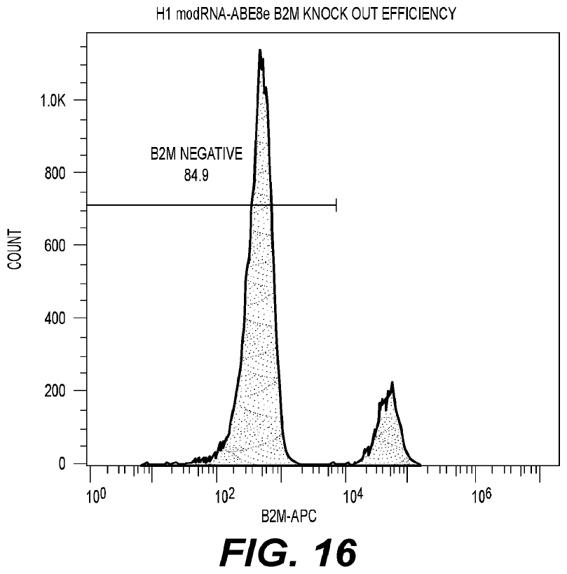

Gene knockout using ABE8e modRNA. After lipofection of the ABE8e-GFP modRNA into hPSCs, we demonstrated that it outperforms the transiently expressed ABE8e DNA plasmid when lipofected using similar methods. This outperformance is likely due to lipofection alone being inefficient at delivering plasmid ABE8e DNA into the nucleus of the hPSCs, where transcription is required as the first step for successful base editing. This leads to lower transcripts/protein per cell and/or less cells total expressing ABE8e, thus generating lower base editing efficiencies. By in vitro transcribing the ABE8e modRNA, it eliminates the need for the cell to perform this transcription, and for higher cytosolic transcript/protein levels, which ultimately results in higher base editing efficiencies. Using the SpliceR program reported by Kluesner et al., which generates gRNAs that can target the splice donor or acceptors of your gene of interest. We decided to target the B2M gene. B2M knock out cell lines have been shown to be immune evasive towards cytotoxic T cell lysis, due to major histocompatibility complex expression being dependent on B2M gene expression. By lipofecting both a gRNA that targets the Exon 1 donor region with of B2M, and by lipofecting the ABE8e-GFP modRNA, we demonstrated a B2M knock out efficiency of about 85% (FIG. 16). This illustrates the utility of modRNA mediated base editing for gene knock out.

### Example 5. ModRNA-Based Cas9 Technology

Cas9-T2A-Puro modRNA. The 5MCS3 plasmid was linearized with EcoRI and SpeI digestion for 1 hour. spCas9-T2A-Puro amplicon was amplified from PB-CRISPR (Addgene #160047) with Q5 polymerase. The plasmid was ligated using In-Fusion ligase (Takara Bio) and transformed into chemically competent Stb13 E. Coli (Thermo Fisher). spCas9-T2A-Puro modRNA was synthesized via in-vitro transcription using the MEGAscript T7 Transcription Kit (Thermo Fisher) with N1-methyl-psuedouridine and ARCA (Anti-Reverse Cap Analog). The IVT reaction product was treated with DnaseI and purified with the MEGAclear Transcription Cleanup Kit (Thermo Fisher).

P53DD modRNA. P53DD amplicon was amplified from the plasmid pCE-mp53DD (Addgene #41856) with Q5 polymerase and ligated into the linearized 5MCS3 backbone with In-Fusion Ligase (Takara Bio). It was then transformed into chemically competent Stb13 E. Coli (Thermo Fisher). P53DD modRNA was synthesized via in-vitro transcription as described for Cas9-T2Apuro modRNA.

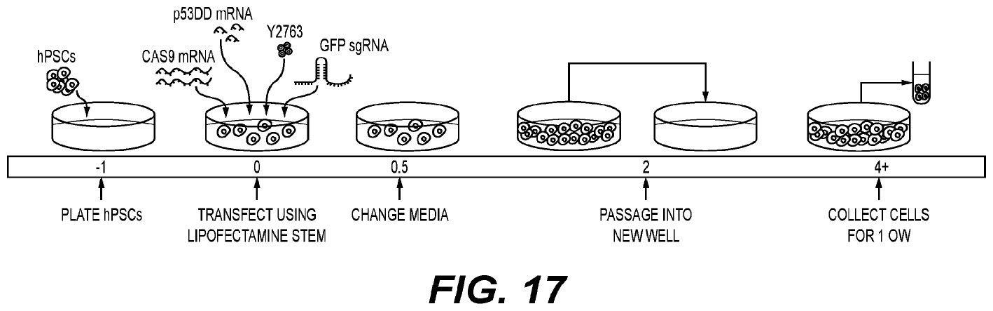

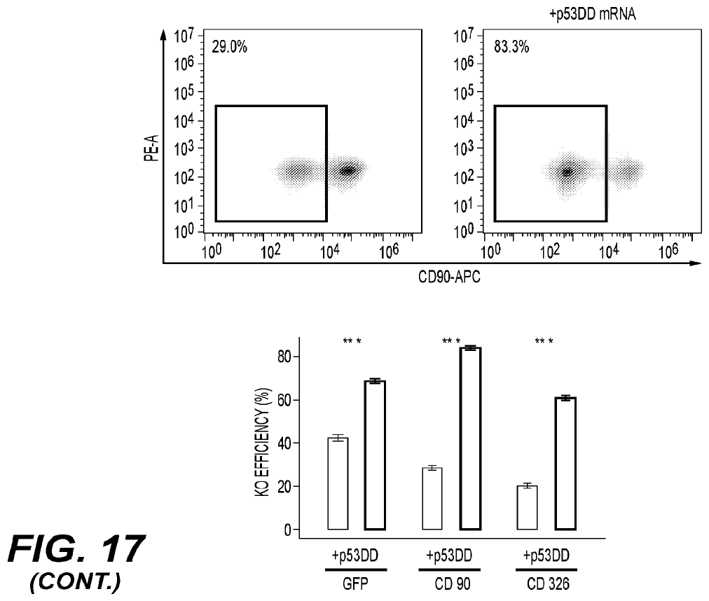

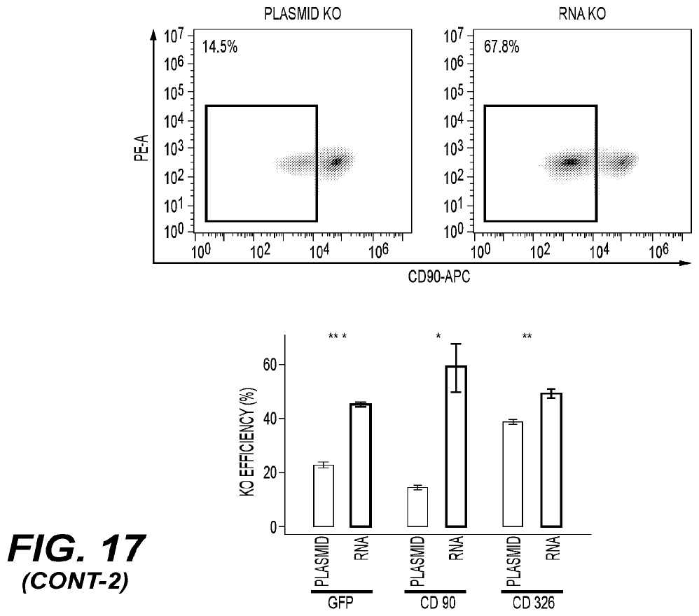

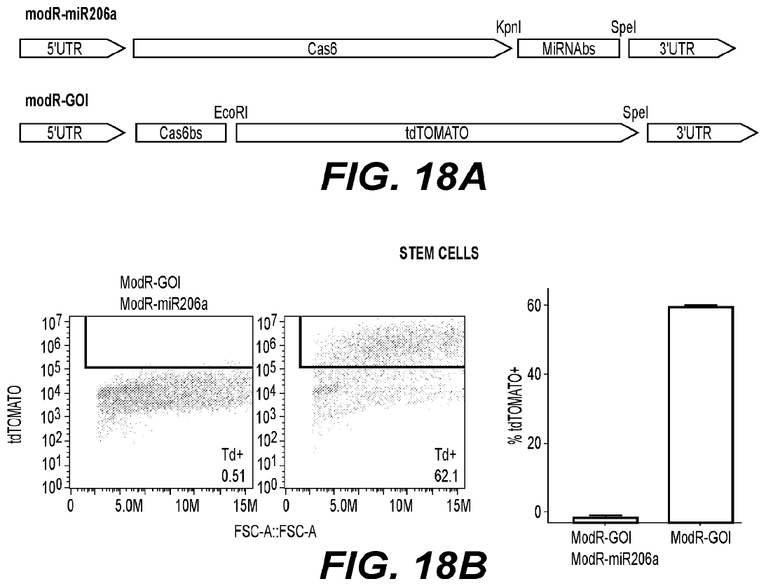

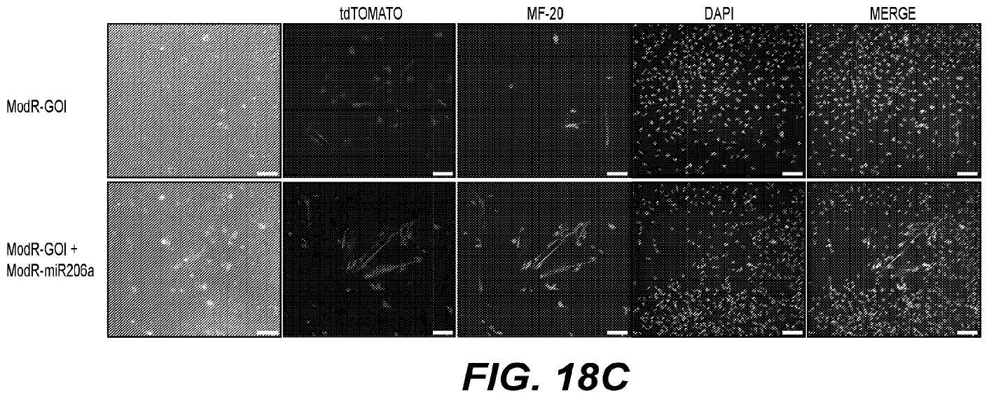

Gene knockout using Cas9 modRNA. Using this Cas9-T2A-Puro modRNA, p53DD modRNA and appropriate sgRNA delivered via lipofection with Lipofectamine Stem Reagent, highly efficient knockout of GFP, CD90, and CD326 was able to achieve in human pluripotent stem cells (hPSCs) (FIG. 17). The results below show that this protocol is superior to previously described methods using only Cas9 modRNA and appropriate sgRNA, as well as state-of-the-art plasmid-based methods.

SEQUENCES

SEQ ID NO: 1 (RNA sequence for Cas9)
auggauuacaaagacgaugacgauaagauggccccaaagaagaagcggaaggucgguauccacggagucccagcagccgacaa
gaaguacagcaucggccuggacaucggcaccaacucugugggcugggccgugaucaccgacgaguacaaggugcccagcaag
aaauucaaggugcugggcaacaccgaccggcacagcaucaagaagaaccugaucggagcccugcuguucgacagcggcgaaac
agccgaggccacccggcugaagagaaccgccagaagaagauacaccagacggaagaaccggaucugcuaucugcaagagaucu
ucagcaacgagauggccaagguggacgacagcuucuuccacagacuggaagaguccuuccugguggaagaggauaagaagca
cgagcggcaccccaucuucggcaacaucguggacgagguggccuaccacgagaaguaccccaccaucuaccaccugagaaaga
aacugguggacagcaccgacaaggccgaccugcggcugaucuaucuggcccuggcccacaugaucaaguuccggggccacuu
ccugaucgagggcgaccugaaccccgacaacagcgacguggacaagcuguucauccagcuggugcagaccuacaaccagcugu
ucgaggaaaaccccaucaacgccagcggcguggacgccaaggccauccugucugccagacugagcaagagcagacggcuggaa
aaucugaucgcccagcugcccggcgagaagaagaauggccuguucggcaaccugauugcccugagccugggccugacccccaa
cuucaagagcaacuucgaccuggccgaggaugccaaacugcagcugagcaaggacaccuacgacgacgaccuggacaaccugc
uggcccagaucggcgaccaguacgccgaccuguuucuggccgccaagaaccuguccgacgccauccugcugagcgacauccug
agagugaacaccgagaucaccaaggccccccugagcgccucuaugaucaagagauacgacgagcaccaccaggaccugacccu
gcugaaagcucucgugcggcagcagcugccugagaaguacaaagagauuuucuucgaccagagcaagaacggcuacgccggc
uacauugacggcggagccagccaggaagaguucuacaaguucaucaagcccauccuggaaaagauggacggcaccgaggaacu
gcucgugaagcugaacagagaggaccugcugcggaagcagcggaccuucgacaacggcagcaucccccaccagauccaccugg
gagagcugcacgccauucugcggcggcaggaagauuuuuacccauuccugaaggacaaccgggaaaagaucgagaagauccu
gaccuuccgcauccccuacuacgugggcccucuggccaggggaaacagcagauucgccuggaugaccagaaagagcgaggaaa
ccaucacccccuggaacuucgaggaagugguggacaagggcgcuuccgcccagagcuucaucgagcggaugaccaacuucga
uaagaaccugcccaacgagaaggugcugcccaagcacagccugcuguacgaguacuucaccguguauaacgagcugaccaaag
ugaaauacgugaccgagggaaugagaaagcccgccuuccugagcggcgagcagaaaaaggccaucguggaccugcuguucaa
gaccaaccggaaagugaccgugaagcagcugaaagaggacuacuucaagaaaaucgagugcuucgacuccguggaaaucuccg
gcguggaagaucgguucaacgccucccugggcacauaccacgaucugcugaaaauuaucaaggacaaggacuuccuggacaau
gaggaaaacgaggacauucuggaagauaucgugcugacccugacacuguuugaggacagagagaugaucgaggaacggcuga
aaaccuaugcccaccuguucgacgacaaagugaugaagcagcugaagcggcggagauacaccggcuggggcaggcugagccg
gaagcugaucaacggcauccgggacaagcaguccggcaagacaauccuggauuuccugaaguccgacggcuucgccaacagaa
acuucaugcagcugauccacgacgacagccugaccuuuaaagaggacauccagaaagcccagguguccggccagggcgauagc
cugcacgagcacauugccaaucuggccggcagccccgccauuaagaagggcauccugcagacagugaaggugguggacgagc
ucgugaaagugaugggccggcacaagcccgagaacaucgugaucgaaauggccagagagaaccagaccacccagaagggacag
aagaacagccgcgagagaaugaagcggaucgaagagggcaucaaagagcugggcagccagauccugaaagaacaccccgugga
aaacacccagcugcagaacgagaagcuguaccuguacuaccugcagaaugggccgggauauguacguggaccaggaacuggaca
ucaaccggcuguccgacuacgauguggaccauaucgugccucagagcuuucugaaggacgacuccaucgacaacaaggugcu
gaccagaagcgacaagaaccggggcaagagcgacaacgugcccuccgaagaggucgugaagaagaugaagaacuacuggcggc
agcugcugaacgccaagcugauuacccagagaaaguucgacaaucugaccaaggccgagagaggcggccugagcgaacuggau
aaggccggcuucaucaagagacagcugguggaaacccggcagaucacaaagcacguggcacagauccuggacucccggaugaa
cacuaaguacgacgagaaugacaagcugauccgggaagugaaagugaucacccugaaguccaagcugguguccgauuuccgg
aaggauuuccaguuuuacaaagugcgcgagaucaacaacuaccaccacgcccacgacgccuaccugaacgccgucgugggaac
cgcccugaucaaaaaguacccuaagcuggaaagcgaguucguguacggcgacuacaagguguacgacgugcggaagaugauc
gccaagagcgagcaggaaaucggcaaggcuaccgccaaguacuucuucuacagcaacaucaugaacuuuuucaagaccgagau
uacccuggccaacggcgagauccggaagcggccucugaucgagacaaacggcgaaaccggggagaucgugugggauaagggc
cgggauuuugccaccgugcggaaagugcugagcaugccccaagugaauaucgugaaaaagaccgaggugcagacaggcggcu
ucagcaaagagucuauccugcccaagaggaacagcgauaagcugaucgccagaaagaaggacugggacccuaagaaguacggc
ggcuucgacagccccaccguggccuauucugugcuggugguggccaaaguggaaaagggcaaguccaagaaacugaagagug
ugaaagagcugcuggggaucaccaucauggaaagaagcagcuucgagaagaaucccaucgacuuucuggaagccaagggcua
caaagaagugaaaaaggaccugaucaucaagcugccuaaguacucccuguucgagcuggaaaacggccggaagagaaugcugg
ccucugccggcgaacugcagaagggaaacgaacuggcccugcccuccaaauaugugaacuuccuguaccuggccagccacuau
gagaagcugaagggcucccccgaggauaaugagcagaaacagcuguuuguggaacagcacaagcacuaccuggacgagaucau
cgagcagaucagcgaguucuccaagagagugauccuggccgacgcuaaucuggacaaagugcuguccgccuacaacaagcacc
gggauaagcccaucagagagcaggccgagaauaucauccaccuguuuacccugaccaaucugggagccccugccgccuucaag
uacuuugacaccaccaucgaccggaagagguacaccagcaccaaagaggugcuggacgccacccugauccaccagagcaucacc
ggccuguacgagacacggaucgaccugucucagcugggaggcgacaagcguccugcugcuacuaagaaagcuggucaagcua
agaaaaagaaa
SEQ ID NO: 2 (RNA sequence for p53DD)
augacugccauggaggagucacagucggauaucagccucaagagagcgcugcccaccugcacaagcgccucucccccgcaaaa
gaaaaaaccacuugauggagaguauuucacccucaagauccgcgggcguaaacgcuucgagauguuccgggagcugaaugag
gccuuagaguuaaaggaugcccaugcuacagaggagucuggagacagcagggcucacuccagcuaccugaagaccaagaagg
gccagucuacuucccgccauaaaaaaacaauggucaagaaaguggggccugacucagacuga
SEQ ID NO: 3 (RNA sequence for base editor (ABE8e)
augaaacggacagccgacggaagcgaguucgagucaccaaagaagaagcggaaagucucugagguggaguuuucccacgagu
acuggaugagacaugcccugacccuggccaagagggcacgggaugagagggaggugccugugggagccgugcuggugcuga
acaauagagugaucggcgagggcuggaacagagccaucggccugcacgacccaacagcccaugccgaaauuauggcccugaga
cagggcggccuggucaugcagaacuacagacugauugacgccacccuguacgugacauucgagccuugcgugaugugcgccg
gcgccaugauccacucuaggaucggccgcgugguguuuggauggagaaauucuaaaagaggcgccgcaggcucccugaugaa
cgugcugaacuaccccggcaugaaucaccgcgucgaaauuaccgagggaauccuggcagaugaaugugccgcccugcugugc
gauuucuaucggaugccuagacagguguucaaugcucagaagaaggcccagagcuccaucaacuccggaggaucuagcggag
gcuccucuggcucugagacaccuggcacaagcgagagcgcaacaccugaaagcagcgggggcagcagcggggggucagacaa
gaaguacagcaucggccuggccaucggcaccaacucugugggcugggccgugaucaccgacgaguacaaggugcccagcaag
aaauucaaggugcugggcaacaccgaccggcacagcaucaagaagaaccugaucggagcccugcuguucgacagcggcgaaac
agccgaggccacccggcugaagagaaccgccagaagaagauacaccagacggaagaaccggaucugcuaucugcaagagaucu
ucagcaacgagauggccaagguggacgacagcuucuuccacagacuggaagaguccuuccugguggaagaggauaagaagca
cgagcggcaccccaucuucggcaacaucguggacgagguggccuaccacgagaaguaccccaccaucuaccaccugagaaaga
aacugguggacagcaccgacaaggccgaccugcggcugaucuaucuggcccuggcccacaugaucaaguuccggggccacuu
ccugaucgagggcgaccugaaccccgacaacagcgacguggacaagcuguucauccagcuggugcagaccuacaaccagcugu
ucgaggaaaaccccaucaacgccagcggcguggacgccaaggccauccugucugccagacugagcaagagcagacggcuggaa
aaucugaucgcccagcugcccggcgagaagaagaauggccuguucggaaaccugauugcccugagccugggccugacccccaa
cuucaagagcaacuucgaccuggccgaggaugccaaacugcagcugagcaaggacaccuacgacgacgaccuggacaaccugc
uggcccagaucggcgaccaguacgccgaccuguuucuggccgccaagaaccuguccgacgccauccugcugagcgacauccug
agagugaacaccgagaucaccaaggccccccugagcgccucuaugaucaagagauacgacgagcaccaccaggaccugacccu
gcugaaagcucucgugcggcagcagcugccugagaaguacaaagagauuuucuucgaccagagcaagaacggcuacgccggc
uacauugacggcggagccagccaggaagaguucuacaaguucaucaagcccauccuggaaaagauggacggcaccgaggaacu
gcucgugaagcugaacagagaggaccugcugcggaagcagcggaccuucgacaacggcagcaucccccaccagauccaccugg
gagagcugcacgccauucugcggcggcaggaagauuuuuacccauuccugaaggacaaccgggaaaagaucgagaagauccu
gaccuuccgcauccccuacuacgugggcccucuggccaggggaaacagcagauucgccuggaugaccagaaagagcgaggaaa
ccaucacccccuggaacuucgaggaagugguggacaagggcgcuuccgcccagagcuucaucgagcggaugaccaacuucga
uaagaaccugcccaacgagaaggugcugcccaagcacagccugcuguacgaguacuucaccguguauaacgagcugaccaaag
ugaaauacgugaccgagggaaugagaaagcccgccuuccugagcggcgagcagaaaaaggccaucguggaccugcuguucaa
gaccaaccggaaagugaccgugaagcagcugaaagaggacuacuucaagaaaaucgagugcuucgacuccguggaaaucuccg
gcguggaagaucgguucaacgccucccugggcacauaccacgaucugcugaaaauuaucaaggacaaggacuuccuggacaau
gaggaaaacgaggacauucuggaagauaucgugcugacccugacacuguuugaggacagagagaugaucgaggaacggcuga
aaaccuaugcccaccuguucgacgacaaagugaugaagcagcugaagcggcggagauacaccggcuggggcaggcugagccg
gaagcugaucaacggcauccgggacaagcaguccggcaagacaauccuggauuuccugaaguccgacggcuucgccaacagaa
acuucaugcagcugauccacgacgacagccugaccuuuaaagaggacauccagaaagcccagguguccggccagggcgauagc
cugcacgagcacauugccaaucuggccggcagccccgccauuaagaagggcauccugcagacagugaaggugguggacgagc
ucgugaaagugaugggccggcacaagcccgagaacaucgugaucgaaauggccagagagaaccagaccacccagaagggacag
aagaacagccgcgagagaaugaagcggaucgaagagggcaucaaagagcugggcagccagauccugaaagaacaccccgugga
aaacacccagcugcagaacgagaagcuguaccuguacuaccugcagaaugggcgggauauguacguggaccaggaacuggaca
ucaaccggcuguccgacuacgauguggaccauaucgugccucagagcuuucugaaggacgacuccaucgacaacaaggugcu
gaccagaagcgacaagaaccggggcaagagcgacaacgugcccuccgaagaggucgugaagaagaugaagaacuacuggcggc
agcugcugaacgccaagcugauuacccagagaaaguucgacaaucugaccaaggccgagagaggcggccugagcgaacuggau
aaggccggcuucaucaagagacagcugguggaaacccggcagaucacaaagcacguggcacagauccuggacucccggaugaa
cacuaaguacgacgagaaugacaagcugauccgggaagugaaagugaucacccugaaguccaagcugguguccgauuuccgg
aaggauuuccaguuuuacaaagugcgcgagaucaacaacuaccaccacgcccacgacgccuaccugaacgccgucgugggaac
cgcccugaucaaaaaguacccuaagcuggaaagcgaguucguguacggcgacuacaagguguacgacgugcggaagaugauc
gccaagagcgagcaggaaaucggcaaggcuaccgccaaguacuucuucuacagcaacaucaugaacuuuuucaagaccgagau
uacccuggccaacggcgagauccggaagcggccucugaucgagacaaacggcgaaaccggggagaucgugugggauaagggc
cgggauuuugccaccgugcggaaagugcugagcaugccccaagugaauaucgugaaaaagaccgaggugcagacaggcggcu
ucagcaaagagucuauccugcccaagaggaacagcgauaagcugaucgccagaaagaaggacugggacccuaagaaguacggc
ggcuucgacagccccaccguggccuauucugugcuggugguggccaaaguggaaaagggcaaguccaagaaacugaagagug
ugaaagagcugcuggggaucaccaucauggaaagaagcagcuucgagaagaaucccaucgacuuucuggaagccaagggcua
caaagaagugaaaaaggaccugaucaucaagcugccuaaguacucccuguucgagcuggaaaacggccggaagagaaugcugg
ccucugccggcgaacugcagaagggaaacgaacuggcccugcccuccaaauaugugaacuuccuguaccuggccagccacuau
gagaagcugaagggcucccccgaggauaaugagcagaaacagcuguuuguggaacagcacaagcacuaccuggacgagaucau
cgagcagaucagcgaguucuccaagagagugauccuggccgacgcuaaucuggacaaagugcuguccgccuacaacaagcacc
gggauaagcccaucagagagcaggccgagaauaucauccaccuguuuacccugaccaaucugggagccccugccgccuucaag
uacuuugacaccaccaucgaccggaagagguacaccagcaccaaagaggugcuggacgccacccugauccaccagagcaucacc
ggccuguacgagacacggaucgaccugucucagcugggaggugacucuggcggcucaaaaagaaccgccgacggcagcgaau
ucgagcccaagaagaagaggaaaguc
SEQ ID NO: 4 (RNA sequence for WPRE)
UCGACAAUCAACCUCUGGAUUACAAAAUUUGUGAAAGAUUGACUGGUAUUCUUA
ACUAUGUUGCUCCUUUUACGCUAUGUGGAUACGCUGCUUUAAUGCCUUUGUAUC
AUGCUAUUGCUUCCCGUAUGGCUUUCAUUUUCUCCUCCUUGUAUAAAUCCUGGU
UGCUGUCUCUUUAUGAGGAGUUGUGGCCCGUUGUCAGGCAACGUGGCGUGGUGU
GCACUGUGUUUGCUGACGCAACCCCCACUGGUUGGGGCAUUGCCACCACCUGUCA
GCUCCUUUCCGGGACUUUCGCUUUCCCCCUCCCUAUUGCCACGGCGGAACUCAUC
GCCGCCUGCCUUGCCCGCUGCUGGACAGGGGCUCGGCUGUUGGGCACUGACAAUU
CCGUGGUGUUGUCGGGGAAGCUGACGUCCUUUCCAUGGCUGCUCGCCUGUGUUGC
CACCUGGAUUCUGCGCGGGACGUCCUUCUGCUACGUCCCUUCGGCCCUCAAUCCA
GCGGACCUUCCUUCCCGCGGCCUGCUGCCGGCUCUGCGGCCUCUUCCGCGUCUUC
GCCUUCGCCCUCAGACGAGUCGGAUCUCCCUUUGGGCCGCCUCCCCGCCUG
SEQ ID NO: 5 (RNA sequence for Cas6)
Augggugaucauuaucuggauauucggcugaggccugauccagaguucccaccugcgcagcugaugucuguccuuuuuggc
aaacuucaucaggcccugguugcccagggggagaucggauagggguaagcuuuccagaccucgacgaaagccggagccgcc
ugggagaacgccugcggauccacgcuucugccgacgaucugagagccuugcuggcaaggccauggcuugaggggcuccggga
ucaccugcaguuuggcgaacccgccguuguuccccacccaaccccuuaucggcaggugucuagagugcaggccaaaucuaauc
cagaacggcugcgacggcgacucaugcggcgacaugaucuuagcgaggaagaggcccgaaaaagaaucccugauaccguggcc
cgcgcccuugacuugccuuuugucacacugcggucccagaguacggggcagcauuucagacuuuucauucgacacgggccac
ugcaaguuaccgccgaagaaggaggcuuuacuuguuauggacucuccaagggagguuucgugcccugguuuUAG
SEQ ID NO: 6 (RNA sequence for miR-206 binding site)
ccacacacuuccuuacauucca
SEQ ID NO: 7 (RNA sequence for CAS6 binding site)
GUUCACUGCCGUAUAGGCAGCU
SEQ ID NO: 8 (DNA template sequence for Cas9)
atggattacaaagacgatgacgataagatggccccaaagaagaagcggaaggtcggtatccacggagtcccagcagccgacaagaagt
acagcatcggcctggacatcggcaccaactctgtgggctgggccgtgatcaccgacgagtacaaggtgcccagcaagaaattcaaggtg
ctgggcaacaccgaccggcacagcatcaagaagaacctgatcggagccctgctgttcgacagcggcgaaacagccgaggccacccgg
ctgaagagaaccgccagaagaagatacaccagacggaagaaccggatctgctatctgcaagagatcttcagcaacgagatggccaaggt
ggacgacagcttcttccacagactggaagagtccttcctggtggaagaggataagaagcacgagcggcaccccatcttcggcaacatcgt
ggacgaggtggcctaccacgagaagtaccccaccatctaccacctgagaaagaaactggtggacagcaccgacaaggccgacctgcg
gctgatctatctggccctggcccacatgatcaagttccggggccacttcctgatcgagggcgacctgaaccccgacaacagcgacgtgga
caagctgttcatccagctggtgcagacctacaaccagctgttcgaggaaaaccccatcaacgccagcggcgtggacgccaaggccatcct
gtctgccagactgagcaagagcagacggctggaaaatctgatcgcccagctgcccggcgagaagaagaatggcctgttcggcaacctga
tttgccctgagcctgggcctgacccccaacttcaagagcaacttcgacctggccgaggatgccaaactgcagctgagcaaggacacciacg
acgacgacctggacaacctgctggcccagatcggcgaccagtacgccgacctgtttctggccgccaagaacctgtccgacgccatcctgc
tgagcgacatcctgagagtgaacaccgagatcaccaaggcccccctgagcgcctctatgatcaagagatacgacgagcaccaccaggac
ctgaccctgctgaaagctctcgtgcggcagcagctgcctgagaagtacaaagagattttcttcgaccagagcaagaacggctacgccggc
tacattgacggcggagccagccaggaagagttctacaagttcatcaagcccatcctggaaaagatggacggcaccgaggaactgctcgtg
aagctgaacagagaggacctgctgcggaagcagcggaccttcgacaacggcagcatcccccaccagatccacctgggagagctgcac
gccattctgcggcggcaggaagatttttacccattcctgaaggacaaccgggaaaagatcgagaagatcctgaccttccgcatcccctacta
cgtgggccctctggccaggggaaacagcagattcgcctggatgaccagaaagagcgaggaaaccatcaccccctggaacttcgaggaa
gtggtggacaagggcgcttccgcccagagcttcatcgagcggatgaccaacttcgataagaacctgcccaacgagaaggtgctgcccaa
gcacagcctgctgtacgagtacttcaccgtgtataacgagctgaccaaagtgaaatacgtgaccgagggaatgagaaagcccgccttcctg
agcggcgagcagaaaaaggccatcgtggacctgctgttcaagaccaaccggaaagtgaccgtgaagcagctgaaagaggactacttca
agaaaatcgagtgcttcgactccgtggaaatctccggcgtggaagatcggttcaacgcctccctgggcacataccacgatctgctgaaaatt
atcaaggacaaggacttcctggacaatgaggaaaacgaggacattctggaagatatcgtgctgaccctgacactgtttgaggacagagag
atgatcgaggaacggctgaaaacctatgcccacctgttcgacgacaaagtgatgaagcagctgaagcggcggagatacaccggctggg
gcaggctgagccggaagctgatcaacggcatccgggacaagcagtccggcaagacaatcctggatttcctgaagtccgacggcttcgcc
aacagaaacttcatgcagctgatccacgacgacagcctgacctttaaagaggacatccagaaagcccaggtgtccggccagggcgatag
cctgcacgagcacattgccaatctggccggcagccccgccattaagaagggcatcctgcagacagtgaaggtggtggacgagctcgtga
aagtgatgggccggcacaagcccgagaacatcgtgatcgaaatggccagagagaaccagaccacccagaagggacagaagaacagc
cgcgagagaatgaagcggatcgaagagggcatcaaagagctgggcagccagatcctgaaagaacaccccgtggaaaacacccagctg
cagaacgagaagctgtacctgtactacctgcagaatggggggatatgtacgtggaccaggaactggacatcaaccggctgtccgactac
gatgtggaccatatcgtgcctcagagctttctgaaggacgactccatcgacaacaaggtgctgaccagaagcgacaagaaccggggcaa
gagcgacaacgtgccctccgaagaggtcgtgaagaagatgaagaactactggcggcagctgctgaacgccaagctgattacccagaga
aagttcgacaatctgaccaaggccgagagaggcggcctgagcgaactggataaggccggcttcatcaagagacagctggtggaaaccc
ggcagatcacaaagcacgtggcacagatcctggactcccggatgaacactaagtacgacgagaatgacaagctgatccgggaagtgaaa
gtgatcaccctgaagtccaagctggtgtccgatttccggaaggatttccagttttacaaagtgcgcgagatcaacaactaccaccacgccca
cgacgcctacctgaacgccgtcgtgggaaccgccctgatcaaaaagtaccctaagctggaaagcgagttcgtgtacggcgactacaaggt
gtacgacgtgcggaagatgatcgccaagagcgagcaggaaatcggcaaggctaccgccaagtacttcttctacagcaacatcatgaacttt
ttcaagaccgagattaccctggccaacggcgagatccggaagcggcctctgatcgagacaaacggcgaaaccggggagatcgtgtggg
ataagggccgggattttgccaccgtgcggaaagtgctgagcatgccccaagtgaatatcgtgaaaaagaccgaggtgcagacaggcggc
ttcagcaaagagtctatcctgcccaagaggaacagcgataagctgatcgccagaaagaaggactgggaccctaagaagtacggcggctt
cgacagccccaccgtggcctattctgtgctggtggtggccaaagtggaaaagggcaagtccaagaaactgaagagtgtgaaagagctgct
ggggatcaccatcatggaaagaagcagcttcgagaagaatcccatcgactttctggaagccaagggctacaaagaagtgaaaaaggacc
tgatcatcaagctgcctaagtactccctgttcgagctggaaaacggccggaagagaatgctggcctctgccggcgaactgcagaagggaa
acgaactggccctgccctccaaatatgtgaacttcctgtacctggccagccactatgagaagctgaagggctcccccgaggataatgagca
gaaacagctgtttgtggaacagcacaagcactacctggacgagatcatcgagcagatcagcgagttctccaagagagtgatcctggccga
cgctaatctggacaaagtgctgtccgcctacaacaagcaccgggataagcccatcagagagcaggccgagaatatcatccacctgtttacc
ctgaccaatctgggagcccctgccgccttcaagtactttgacaccaccatcgaccggaagaggtacaccagcaccaaagaggtgctggac
gccaccctgatccaccagagcatcaccggcctgtacgagacacggatcgacctgtctcagctgggaggcgacaagcgtcctgctgctact
aagaaagctggtcaagctaagaaaaagaaa
SEQ ID NO: 9 (DNA template sequence for p53DD)
atgactgccatggaggagtcacagtcggatatcagcctcaagagagcgctgcccacctgcacaagcgcctctcccccgcaaaagaaaaa
accacttgatggagagtatttcaccctcaagatccgcgggcgtaaacgcttcgagatgttccgggagctgaatgaggccttagagttaaagg
atgcccatgctacagaggagtctggagacagcagggctcactccagctacctgaagaccaagaagggccagtctacttcccgccataaaa
aaacaatggtcaagaaagtggggcctgactcagactga
SEQ ID NO: 10 (DNA template sequence for base editor (ABE8e)
atgaaacggacagccgacggaagcgagttcgagtcaccaaagaagaagcggaaagtctctgaggtggagttttcccacgagtactggat
gagacatgccctgaccctggccaagagggcacgggatgagagggaggtgcctgtgggagccgtgctggtgctgaacaatagagtgatc
ggcgagggctggaacagagccatcggcctgcacgacccaacagcccatgccgaaattatggccctgagacagggcggcctggtcatgc
agaactacagactgattgacgccaccctgtacgtgacattcgagccttgcgtgatgtgcgccggcgccatgatccactctaggatcggccg
cgtggtgtttggatggagaaattctaaaagaggcgccgcaggctccctgatgaacgtgctgaactaccccggcatgaatcaccgcgtcgaa
attaccgagggaatcctggcagatgaatgtgccgccctgctgtgcgatttctatcggatgcctagacaggtgttcaatgctcagaagaaggc
ccagagctccatcaactccggaggatctagcggaggctcctctggctctgagacacctggcacaagcgagagcgcaacacctgaaagca
gcgggggcagcagcggggggtcagacaagaagtacagcatcggcctggccatcggcaccaactctgtgggctgggccgtgatcaccg
acgagtacaaggtgcccagcaagaaattcaaggtgctgggcaacaccgaccggcacagcatcaagaagaacctgatcggagccctgct
gttcgacagcggcgaaacagccgaggccacccggctgaagagaaccgccagaagaagatacaccagacggaagaaccggatctgcta
tctgcaagagatcttcagcaacgagatggccaaggtggacgacagcttcttccacagactggaagagtccttcctggtggaagaggataag
aagcacgagcggcaccccatcttcggcaacatcgtggacgaggtggcctaccacgagaagtaccccaccatctaccacctgagaaagaa
actggtggacagcaccgacaaggccgacctgcggctgatctatctggccctggcccacatgatcaagttccggggccacttcctgatcga
gggcgacctgaaccccgacaacagcgacgtggacaagctgttcatccagctggtgcagacctacaaccagctgttcgaggaaaacccca
tcaacgccagcggcgtggacgccaaggccatcctgtctgccagactgagcaagagcagacggctggaaaatctgatcgcccagctgcc
cggcgagaagaagaatggcctgttcggaaacctgattgccctgagcctgggcctgacccccaacttcaagagcaacttcgacctggccga
ggatgccaaactgcagctgagcaaggacacctacgacgacgacctggacaacctgctggcccagatcggcgaccagtacgccgacctg
tttctggccgccaagaacctgtccgacgccatcctgctgagcgacatcctgagagtgaacaccgagatcaccaaggcccccctgagcgcc
tctatgatcaagagatacgacgagcaccaccaggacctgaccctgctgaaagctctcgtgcggcagcagctgcctgagaagtacaaaga
gattttcttcgaccagagcaagaacggctacgccggctacattgacggcggagccagccaggaagagttctacaagttcatcaagcccatc
ctggaaaagatggacggcaccgaggaactgctcgtgaagctgaacagagaggacctgctgcggaagcagcggaccttcgacaacggc
agcatcccccaccagatccacctgggagagctgcacgccattctgcggcggcaggaagatttttacccattcctgaaggacaaccgggaa
aagatcgagaagatcctgaccttccgcatcccctactacgtgggccctctggccaggggaaacagcagattcgcctggatgaccagaaag
agcgaggaaaccatcaccccctggaacttcgaggaagtggtggacaagggcgcttccgcccagagcttcatcgagcggatgaccaactt
cgataagaacctgcccaacgagaaggtgctgcccaagcacagcctgctgtacgagtacttcaccgtgtataacgagctgaccaaagtgaa
atacgtgaccgagggaatgagaaagcccgccttcctgagcggcgagcagaaaaaggccatcgtggacctgctgttcaagaccaaccgg
aaagtgaccgtgaagcagctgaaagaggactacttcaagaaaatcgagtgcttcgactccgtggaaatctccggcgtggaagatcggttca
acgcctccctgggcacataccacgatctgctgaaaattatcaaggacaaggacttcctggacaatgaggaaaacgaggacattctggaag
atatcgtgctgaccctgacactgtttgaggacagagagatgatcgaggaacggctgaaaacctatgcccacctgttcgacgacaaagtgat
gaagcagctgaagcggcggagatacaccggctggggcaggctgagccggaagctgatcaacggcatccgggacaagcagtccggca
agacaatcctggatttcctgaagtccgacggcttcgccaacagaaacttcatgcagctgatccacgacgacagcctgacctttaaagagga
catccagaaagcccaggtgtccggccagggcgatagcctgcacgagcacattgccaatctggccggcagccccgccattaagaagggc
atcctgcagacagtgaaggtggtggacgagctcgtgaaagtgatgggccggcacaagcccgagaacatcgtgatcgaaatggccagag
agaaccagaccacccagaagggacagaagaacagccgcgagagaatgaagcggatcgaagagggcatcaaagagctgggcagcca
gatcctgaaagaacaccccgtggaaaacacccagctgcagaacgagaagctgtacctgtactacctgcagaatggggggatatgtacgt
ggaccaggaactggacatcaaccggctgtccgactacgatgtggaccatatcgtgcctcagagctttctgaaggacgactccatcgacaac
aaggtgctgaccagaagcgacaagaaccggggcaagagcgacaacgtgccctccgaagaggtcgtgaagaagatgaagaactactgg
cggcagctgctgaacgccaagctgattacccagagaaagttcgacaatctgaccaaggccgagagaggcggcctgagcgaactggata
aggccggcttcatcaagagacagctggtggaaacccggcagatcacaaagcacgtggcacagatcctggactcccggatgaacactaag
tacgacgagaatgacaagctgatccgggaagtgaaagtgatcaccctgaagtccaagctggtgtccgatttccggaaggatttccagtttta
caaagtgcgcgagatcaacaactaccaccacgcccacgacgcctacctgaacgccgtcgtgggaaccgccctgatcaaaaagtacccta
agctggaaagcgagttcgtgtacggcgactacaaggtgtacgacgtgcggaagatgatcgccaagagcgagcaggaaatcggcaaggc
taccgccaagtacttcttctacagcaacatcatgaactttttcaagaccgagattaccctggccaacggcgagatccggaagcggcctctgat
cgagacaaacggcgaaaccggggagatcgtgtgggataagggccgggattttgccaccgtgcggaaagtgctgagcatgccccaagtg
aatatcgtgaaaaagaccgaggtgcagacaggcggcttcagcaaagagtctatcctgcccaagaggaacagcgataagctgatcgccag
aaagaaggactgggaccctaagaagtacggcggcttcgacagccccaccgtggcctattctgtgctggtggtggccaaagtggaaaagg
gcaagtccaagaaactgaagagtgtgaaagagctgctggggatcaccatcatggaaagaagcagcttcgagaagaatcccatcgactttct
ggaagccaagggctacaaagaagtgaaaaaggacctgatcatcaagctgcctaagtactccctgttcgagctggaaaacggccggaaga
gaatgctggcctctgccggcgaactgcagaagggaaacgaactggccctgccctccaaatatgtgaacttcctgtacctggccagccacta
tgagaagctgaagggctcccccgaggataatgagcagaaacagctgtttgtggaacagcacaagcactacctggacgagatcatcgagc
agatcagcgagttctccaagagagtgatcctggccgacgctaatctggacaaagtgctgtccgcctacaacaagcaccgggataagccca
tcagagagcaggccgagaatatcatccacctgtttaccctgaccaatctgggagcccctgccgccttcaagtactttgacaccaccatcgac
cggaagaggtacaccagcaccaaagaggtgctggacgccaccctgatccaccagagcatcaccggcctgtacgagacacggatcgacc
tgtctcagctgggaggtgactctggcggctcaaaaagaaccgccgacggcagcgaattcgagcccaagaagaagaggaaagtc
SEQ ID NO: 11 (DNA template sequence for WPRE)
TCGACAATCAACCTCTGGATTACAAAATTTGTGAAAGATTGACTGGTATTCTTAACT
ATGTTGCTCCTTTTACGCTATGTGGATACGCTGCTTTAATGCCTTTGTATCATGCTAT
TGCTTCCCGTATGGCTTTCATTTTCTCCTCCTTGTATAAATCCTGGTTGCTGTCTCTTT
ATGAGGAGTTGTGGCCCGTTGTCAGGCAACGTGGCGTGGTGTGCACTGTGTTTGCTG
ACGCAACCCCCACTGGTTGGGGCATTGCCACCACCTGTCAGCTCCTTTCCGGGACTT
TCGCTTTCCCCCTCCCTATTGCCACGGCGGAACTCATCGCCGCCTGCCTTGCCCGCTG
CTGGACAGGGGCTCGGCTGTTGGGCACTGACAATTCCGTGGTGTTGTCGGGGAAGC
TGACGTCCTTTCCATGGCTGCTCGCCTGTGTTGCCACCTGGATTCTGCGCGGGACGT
CCTTCTGCTACGTCCCTTCGGCCCTCAATCCAGCGGACCTTCCTTCCCGCGGCCTGCT
GCCGGCTCTGCGGCCTCTTCCGCGTCTTCGCCTTCGCCCTCAGACGAGTCGGATCTC
CCTTTGGGCCGCCTCCCCGCCTG
SEQ ID NO: 12 (DNA template sequence for Cas6)
Atgggtgatcattatctggatattcggctgaggcctgatccagagttcccacctgcgcagctgatgtctgtcctttttggcaaacttcatca
ggccctggttgcccagggcggagatcggataggggtaagctttccagacctcgacgaaagccggagccgcctgggagaacgcctgcggat
ccacgcttctgccgacgatctgagagccttgctggcaaggccatggcttgaggggctccgggatcacctgcagtttggcgaacccgccgtt
gttccccacccaaccccttatcggcaggtgtctagagtgcaggccaaatctaatccagaacggctgcgacggcgactcatgcggcgacat
gatcttagcgaggaagaggcccgaaaaagaatccctgataccgtggcccgcgcccttgacttgccttttgtcacactgcggtcccagagta
cggggcagcatttcagacttttcattcgacacgggccactgcaagttaccgccgaagaaggaggctttacttgttatggactctccaaggga
ggtttcgtgccctggtttTAG
SEQ ID NO: 13 (DNA template sequence for 4x miR-206 binding site)
ccacacacttccttacattcca
SEQ ID NO: 14 (DNA template sequence for CAS6 binding site)
GTTCACTGCCGTATAGGCAGCT
SEQ ID NO: 15 Full sequence and map of plasmid XloneV3-ABE8e. Related to the STAR
Methods. (DNA)
Full sequence (TRE3G promoter (bolded), ABE8e, 2A (ALL CAPS), EGFP (all CAPS and
UNDERLINED); Tet-On 3G (all CAPS and bolded), EF1a core promoter (bolded and
underlined)):
Atcacctcgagtttactccctatcagtgatagagaacgtatgaagagtttactccctatcagtgatagagaacgtatgcagactttac
tccctatcagtgatagagaacgtataaggagtttactccctatcagtgatagagaacgtatgaccagtttactccctatcagtgatag
agaacgtatctacagtttactccctatcagtgatagagaacgtatatccagtttactccctatcagtgatagagaacgtataagcttt
gcttatgtaaaccagggcgcctataaaagagtgctgattttttgagtaaacttcaattccacaacacttttgtcttataccaactttccg
taccacttcctaccctcgtaaaggtaccgccaccatgaaacggacagccgacggaagcgagttcgagtcaccadagaagaagcgg
aaagtctctgaggtggagttttcccacgagtactggatgagacatgccctgaccctggccaagagggcacgggatgagagggaggtg
cctgtgggagccgtgctggtgctgaacaatagagtgatcggcgagggctggaacagagccatcggcctgcacgacccaacagccca
tgccgaaattatggccctgagacagggcggcctggtcatgcagaactacagactgattgacgccaccctgtacgtgacattcgagcctt
gcgtgatgtgcgccggcgccatgatccactctaggatcggccgcgtggtgtttggatggagaaattctaaaagaggcgccgcaggctc
cctgatgaacgtgctgaactaccccggcatgaatcaccgcgtcgaaattaccgagggaatcctggcagatgaatgtgccgccctgctg
tgcgatttctatcggatgcctagacaggtgttcaatgctcagaagaaggcccagagctccatcaactccggaggatctagcggaggct
cctctggctctgagacacctggcacaagcgagagcgcaacacctgaaagcagcgggggcagcagcggggggtcagacaagaagt
acagcatcggcctggccatcggcaccaacicigtgggcigggccgtgatcaccgacgagtacaaggigcccagcaagaaattcaag
gtgctgggcaacaccgaccggcacagcatcaagaagaacctgatcggagccctgctgttcgacagcggcgaaacagccgaggcc
acccggctgaagagaaccgccagaagaagatacaccagacggaagaaccggatctgctatctgcaagagatcttcagcaacgag
atggccaaggtggacgacagcttcttccacagactggaagagtccttcctggtggaagaggataagaagcacgagcggcaccccat
cttcggcaacatcgtggacgaggtggcctaccacgagaagtaccccaccatctaccacctgagaaagaaactggtggacagcaccg
acaaggccgacctgcggctgatctatctggccctggcccacatgatcaagttccggggccacttcctgatcgagggcgacctgaaccc
cgacaacagcgacgtggacaagctgttcatccagctggtgcagacctacaaccagctgttcgaggaaaaccccatcaacgccagcg
gcgtggacgccaaggccatcctgtctgccagactgagcaagagcagacggctggaaaatctgatcgcccagctgcccggcgagaa
gaagaatggcctgttcggaaacctgattgccctgagcctgggcctgacccccaacttcaagagcaacttcgacctggccgaggatgcc
aaactgcagctgagcaaggacacctacgacgacgacctggacaacctgctggcccagatcggcgaccagtacgccgacctgtttct
ggccgccaagaacctgtccgacgccatcctgctgagcgacatcctgagagtgaacaccgagatcaccaaggcccccctgagcgcct
ctatgatcaagagatacgacgagcaccaccaggacctgaccctgctgaaagctctcgtgcggcagcagctgcctgagaagtacaaa
gagattttcttcgaccagagcaagaacggctacgccggctacattgacggcggagccagccaggaagagttctacaagttcatcaag
cccatcctggaaaagatggacggcaccgaggaactgctcgtgaagctgaacagagaggacctgctgcggaagcagcggaccttcg
acaacggcagcatcccccaccagatccacctgggagagctgcacgccattctgcggcggcaggaagatttttacccattcctgaagg
acaaccgggaaaagatcgagaagatcctgaccttccgcatcccctactacgtgggccctctggccaggggaaacagcagattcgcct
ggatgaccagaaagagcgaggaaaccatcaccccctggaacttcgaggaagtggtggacaagggcgcttccgcccagagcttcat
cgagcggatgaccaacttcgataagaacctgcccaacgagaaggtgctgcccaagcacagcctgctgtacgagtacttcaccgtgta
taacgagctgaccaaagtgaaatacgtgaccgagggaatgagaaagcccgccttcctgagcggcgagcagaaaaaggccatcgtg
gacctgctgttcaagaccaaccggaaagtgaccgtgaagcagctgaaagaggactacttcaagaaaatcgagtgcttcgactccgtg
gaaatctccggcgtggaagatcggttcaacgcctccctgggcacataccacgatctgctgaaaattatcaaggacaaggacttcctgg
acaatgaggaaaacgaggacattctggaagatatcgtgctgaccctgacactgtttgaggacagagagatgatcgaggaacggctg
aaaacctatgcccacctgttcgacgacaaagtgatgaagcagctgaagcggcggagatacaccggctggggcaggctgagccgga
agctgatcaacggcatccgggacaagcagtccggcaagacaatcctggatttcctgaagtccgacggcttcgccaacagaaacttca
tgcagctgatccacgacgacagcctgacctttaaagaggacatccagaaagcccaggtgtccggccagggcgatagcctgcacgag
cacattgccaatctggccggcagccccgccattaagaagggcatcctgcagacagtgaaggtggtggacgagctcgtgaaagtgatg
ggccggcacaagcccgagaacatcgtgatcgaaatggccagagagaaccagaccacccagaagggacagaagaacagccgcg
agagaatgaagcggatcgaagagggcatcaaagagctgggcagccagatcctgaaagaacaccccgtggaaaacacccagctg
cagaacgagaagctgtacctgtactacctgcagaatgggcgggatatgtacgtggaccaggaactggacatcaaccggctgtccgac
tacgatgtggaccatatcgtgcctcagagctttctgaaggacgactccatcgacaacaaggtgctgaccagaagcgacaagaaccgg
ggcaagagcgacaacgtgccctccgaagaggtcgtgaagaagatgaagaactactggcggcagctgctgaacgccaagctgatta
cccagagaaagttcgacaatctgaccaaggccgagagaggcggcctgagcgaactggataaggccggcttcatcaagagacagct
ggtggaaacccggcagatcacaaagcacgtggcacagatcctggactcccggatgaacactaagtacgacgagaatgacaagctg
atccgggaagtgaaagtgatcaccctgaagtccaagctggtgtccgatttccggaaggatttccagttttacaaagtgcgcgagatcaa
caactaccaccacgcccacgacgcctacctgaacgccgtcgtgggaaccgccctgatcaaaaagtaccctaagctggaaagcgagt
tcgtgtacggcgactacaaggtgtacgacgtgcggaagatgatcgccaagagcgagcaggaaatcggcaaggctaccgccaagta
cttcttctacagcaacatcatgaactttttcaagaccgagattaccctggccaacggcgagatccggaagcggcctctgatcgagacaa
acggcgaaaccggggagatcgtgtgggataagggccgggattttgccaccgtgcggaaagtgctgagcatgccccaagtgaatatc
gtgaaaaagaccgaggtgcagacaggcggcttcagcaaagagtctatcctgcccaagaggaacagcgataagctgatcgccagaa
agaaggactgggaccctaagaagtacggcggcttcgacagccccaccgtggcctattctgtgctggtggtggccaaagtggaaaag
ggcaagtccaagaaactgaagagtgtgaaagagctgctggggatcaccatcatggaaagaagcagcttcgagaagaatcccatcg
actttctggaagccaagggctacaaagaagtgaaaaaggacctgatcatcaagctgcctaagtactccctgttcgagctggaaaacg
gccggaagagaatgctggcctctgccggcgaactgcagaagggaaacgaactggccctgccctccaaatatgtgaacttcctgtacc
tggccagccactatgagaagctgaagggctcccccgaggataatgagcagaaacagctgtttgtggaacagcacaagcactacctg
gacgagatcatcgagcagatcagcgagttctccaagagagtgatcctggccgacgctaatctggacaaagtgctgtccgcctacaac
aagcaccgggataagcccatcagagagcaggccgagaatatcatccacctgtttaccctgaccaatctgggagcccctgccgccttc
aagtactttgacaccaccatcgaccggaagaggtacaccagcaccaaagaggtgctggacgccaccctgatccaccagagcatcac
cggcctgtacgagacacggatcgacctgtctcagctgggaggtgactctggcggctcaaaaagaaccgccgacggcagcgaattcg
agcccaagaagaagaggaaagtcgctagcggcagcggcGCCACTAACTTCTCCCTGTTGAAACAAGCA
GGGGATGTCGAAGAGAATCCCGGGCCAATGGTGAGCAAGGGCGAGGAGCTGTTCA
CCGGGGTGGTGCCCATCCTGGTCGAGCTGGACGGCGACGTAAACGGCCACAAGTTC
AGCGTGTCCGGCGAGGGCGAGGGCGATGCCACCTACGGCAAGCTGACCCTGAAGTT
CATCTGCACCACCGGCAAGCTGCCCGTGCCCTGGCCCACCCTCGTGACCACCCTGAC
CTACGGCGTGCAGTGCTTCAGCCGCTACCCCGACCACATGAAGCAGCACGACTTCTT
CAAGTCCGCCATGCCCGAAGGCTACGTCCAGGAGCGCACCATCTTCTTCAAGGACG
ACGGCAACTACAAGACCCGCGCCGAGGTGAAGTTCGAGGGCGACACCCTGGTGAAC
CGCATCGAGCTGAAGGGCATCGACTTCAAGGAGGACGGCAACATCCTGGGGCACAA
GCTGGAGTACAACTACAACAGCCACAACGTCTATATCATGGCCGACAAGCAGAAGA
ACGGCATCAAGGTGAACTTCAAGATCCGCCACAACATCGAGGACGGCAGCGTGCAG
CTCGCCGACCACTACCAGCAGAACACCCCCATCGGCGACGGCCCCGTGCTGCTGCC
CGACAACCACTACCTGAGCACCCAGTCCGCCCTGAGCAAAGACCCCAACGAGAAGC
GCGATCACATGGTCCTGCTGGAGTTCGTGACCGCCGCCGGGATCACTCTCGGCATGG
ACGAGCTGTACAAGCCAAAGAAGAAGCGTAAGGTATAAgtacaagtaagaattccgatcatattcaat
aacccactagtagaccacctcccctgcgagctaagctggacagccaatgacgggtaagagagtgacatttttcactaacctaagacaggag
ggccgtcagagctactgcctaatccaaagacgggtaaaagtgataaaaatgtatcactccaacctaagacaggcgcagcttccgagggatt
tgagatccagacatgataagatacattgatgagtttggacaaaccaaaactagaatgcagtgaaaaaaatgccttatttgtgaaatttgtgat
gctattgccttatttgtaaccattataagctgcaataaacaagtttgatatctataacaagaaaatatatatataataagttatcacgtaagt
agaacatgaaataacaatataattatcgtatgagttaaatcttaaaagtcacgtaaaagataatcatgcgtcattttgactcacgcggtcgtt
atagttcaaaatcagtgacacttaccgcattgacaagcacgcctcacgggagctccaagcggcgactgagatgtcctaaatgcacagcgacgg
attcgcgctatttagaaagagagagcaatatttcaagaatgcatgcgtcaattttacgcagactatctttctagggttaagaattcactggcc
gtcgttttacaacgtcgtgactgggaaaaccctggcgttacccaacttaatcgccttgcagcacatccccctttcgccagctggcgtaatagc
gaagaggcccgcaccgatcgcccttcccaacagttgcgcagcctgaatggcgaatggcgcctgatgcggtattttctccttacgcatctgtgc
ggtatttcacaccgcatatggtgcactctcagtacaatctgctctgatgccgcatagttaagccagccccgacacccgccaacacccgctgac
gcgccctgacgggcttgtctgctcccggcatccgcttacagacaagctgtgaccgtctccgggagctgcatgtgtcagaggttttcaccgtca
tcaccgaaacgcgcgagacgaaagggcctcgtgatacgcctatttttataggttaatgtcatgataataatggtttcttagacgtcaggtggc
acttttcggggaaatgtgcgcggaacccctatttgtttatttttctaaatacattcaaatatgtatccgctcatgagacaataaccctgataa
atgcttcaataatattgaaaaaggaagagtatgagtattcaacatttccgtgtcgcccttattcccttttttgcggcattttgccttcctgtt
tttgctcacccagaaacgctggtgaaagtaaaagatgctgaagatcagttgggtgcacgagtgggttacatcgaactggatctcaacagcggt
aagatccttgagagttttcgccccgaagaacgttttccaatgatgagcacttttaaagttctgctatgtggcgcggtattatcccgtattgac
gccgggcaagagcaactcggtcgccgcatacactattctcagaatgacttggttgagtactcaccagtcacagaaaagcatcttacggatggc
atgacagtaagagaattatgcagtgctgccataaccatgagtgataacactgcggccaacttacttctgacaacgatcggaggaccgaaggag
ctaaccgcttttttgcacaacatgggggatcatgtaactcgccttgatcgttgggaaccggagctgaatgaagccataccaaacgacgagcgt
gacaccacgatgcctgtagcaatggcaacaacgttgcgcaaactattaactggcgaactacttactctagcttcccggcaacaattaatagac
tggatggaggcggataaagttgcaggaccacttctgcgctcggcccttccggctggctggtttattgctgataaatctggagccggtgagcgt
gggtctcgcggtatcattgcagcactggggccagatggtaagccctcccgtatcgtagttatctacacgacggggagtcaggcaactatggat
gaacgaaatagacagatcgctgagataggtgcctcactgattaagcattggtaactgtcagaccaagtttactcatatatactttagattgat
ttaaaacttcatttttaatttaaaaggatctaggtgaagatcctttttgataatctcatgaccaaaatcccttaacgtgagttttcgttccac
tgagcgtcagaccccgtagaaaagatcaaaggatcttcttgagatcctttttttctgcgcgtaatctgctgcttgcaaacaaaaaaaccaccg
ctaccagcggtggtttgtttgccggatcaagagctaccaactctttttccgaaggtaactggcttcagcagagcgcagataccaaatactgtt
cttctagtgtagccgtagttaggccaccacttcaagaactctgtagcaccgcctacatacctcgctctgctaatcctgttaccagtggctgct
gccagtggcgataagtcgtgtcttaccgggttggactcaagacgatagttaccggataaggcgcagcggtcgggctgaacggggggttcgtgc
acacagcccagcttggagcgaacgacctacaccgaactgagatacctacagcgtgagctatgagaaagcgccacgcttcccgaagggagaaag
gcggacaggtatccggtaagcggcagggtcggaacaggagagcgcacgagggagcttccagggggaaacgcctggtatctttatagtcctgtc
gggtttcgccacctctgacttgagcgtcgatttttgtgatgctcgtcaggggggcggagcctatggaaaaacgccagcaacgcggccttttta
cggttcctggccttttgctggccttttgctcacatgttctttcctgcgttatcccctgattctgtggataaccgtattaccgcctttgagtga
gctgataccgctcgccgcagccgaacgaccgagcgcagcgagtcagtgagcgaggaagcggaagagcgcccaatacgcaaaccgcctctcccc
gcgcgttggccgattcattaatgcagctggcacgacaggtttcccgactggaaagcgggcagtgagcgcaacgcaattaatgtgagttagctc
actcattaggcaccccaggctttacactttatgcttccggctcgtatgttgtgtggaattgtgagcggataacaatttcacacaggaaacagc
tatgaccatgattacgccaaggtcgacttaaccctagaaagataatcatattgtgacgtacgttaaagataatcatgcgtaaaattgacgcat
gtgttttatcggtctgtatatcgaggtttatttattaatttgaatagatattaagttttattatatttacacttacatactaataataaattc
aacaaacaatttatttatgtttatttatttattaaaaaaaaacaaaaactcaaaatttcttctataaagtaacaaaacttttagcagtgaaaa
aaatgctttatttgtgaaatttgtgatgctattgctttatttgtaaccattataagctgcaataaacaagttaactaaacgggccctctagat
caggcaccgggcttgcgggtcatgcaccaggtgcgcggtccttcgggcacctcgacgtcggcggtgacggtgaagccgagccgctcgtagaag
gggaggttgcggggcgcggaggtctccaggaagggggcaccccggcgcgctcggccgcctccactccggggagcacgacggcgctgcccagac
ccttgccctggtggtcgggcgagacgccgacggtggccaggaaccacgcgggctccttgggccggtgcggcgccaggaggccttccatctgtt
gctgcgcggccagccgggaaccgctcaactcggccatgcgcgggccgatctcggcgaacaccgcccccgcttcgacgctctccggcgtggtcc
agaccgccaccgcggcgccgtcgtccgcgacccacaccttgccgatgtcgagcccgacgcgcgtgaggaagagttcttgcagctcggtgaccc
gctcgatgtggcggtccggatcgacggtgtggcgcgtggcggggtagtcggcgaacgcggcggcgagggtgcgtacggccctgggga
cgtcgtcgcgggtggcgaggcgcaccgtgggcttgtactcggtcatctcgagcctagggccgggattctcctccacgtcaccgcatgttag
aagacttcctctgccctctccgctgccCCCGGGGAGCATGTCAAGGTCAAAATCGTCAAGAGCGT
CAGCAGGCAGCATATCAAGGTCAAAGTCGTCAAGGGCATCGGCTGGGAGCATG
TCTAAGTCAAAATCGTCAAGGGCGTCGGTCGGCCCGCCGCTTTCGCACTTTAG
CTGTTTCTCCAGGCCACATATGATTAGTTCCAGGCCGAAAAGGAAGGCAGGTT
CGGCTCCCTGCCGGTCGAACAGCTCAATTGCTTGTTTCAGAAGTGGGGGCATA
GAATCGGTGGTAGGTGTCTCTCTTTCCTCTTTTGCTACTTGATGCTCCTGTTCC
TCCAATACGCAGCCCAGTGTAAAGTGGCCCACGGCGGACAGAGCGTACAGTGC
GTTCTCCAGGGAGAAGCCTTGCTGACACAGGAACGCGAGCTGATTTTCCAGGG
TTTCGTACTGTTTCTCTGTTGGGCGGGTGCCGAGATGCACTTTAGCCCCGTCGC
GATGTGAGAGGAGAGCACAGCGGTATGACTTGGCGTTGTTCCGCAGAAAGTCT
TGCCATGACTCGCCTTCCAGGGGGCAGGAGTGGGTATGATGCCTGTCCAGCAT
CTCGATTGGCAGGGCATCGAGCAGGGCCCGCTTGTTCTTCACGTGCCAGTACA
GGGTAGGCTGCTCAACTCCCAGCTTTTGAGCGAGTTTCCTTGTCGTCAGGCCTT
CGATACCGACTCCATTGAGTAATTCCAGAGCAGAGTTTATGACTTTGCTCTTGT
CCAGTCTAGACATcttatcgtcatcgtctttgtaatccatggtggcggatcccgcgtcacgacacctgtgttctggcggcaaa
cccgttgcgaaaaagaacgttcacggcgactactgcacttatatacggttctcccccaccctcgggaaaaaggcggagccagtac
acgacatcactttcccagtttaccccgcgccaccttctctaggcaccggttcaattgccgacccctccccccaacttctcggggactgt
gggcgatgtgcgctctgcccactgacgggcaccggagccactcgagtggaatt
SEQ ID NO: 16 (DNA)
GCCTGCCTAGTGGACCAGAGCCTTCGTCTGGACTGCCGCCATG
SEQ ID NO: 17 (DNA)
GCCTGCCTAGTGGACTGCCGCCATG
SEQ ID NO: 18 (DNA)
GCCTGCCTAGTGGACCAGAGCCTTGAGAATTCTCATTGGACTGCCGCCATG
SEQ ID NO: 19 (DNA)
GCCTGCCTAGTGGACCAGAGCCTTCTGGACTGCCGCCATG
SEQ ID NO: 20 (DNA)
GCCTGCCTAGTGGACCAGAGCCATG
SEQ ID NO: 21 (DNA)
GCCTGCCTAGTGGACCAGAGCCTTCGTTCTGGACTGCCGCCATG
SEQ ID NO: 22 (DNA)
GGCTATCCAGCGTGAGTCTCTCC
SEQ ID NO: 23 (DNA)
GGCTATCCAGCGcGAGcCTCTCC
SEQ ID NO: 24 (DNA)
GGCTATCCAGCGcGAGTCTCTCC
SEQ ID NO: 25 (DNA)
TGAATGACACCATGCAGCCCCGCCCATGGGCCCTCGTCTGGACTGCCTCTTTC
SEQ ID NO: 26 (DNA)
CAGAGCTGCAGTGCAGACGAGGGTTGGGCACCTCAGAGCTGCAGTGCAGACAG
SEQ ID NO: 27 (DNA)
CTTTTTATATGTTGTGTCTCTGATGATTTTCCCTCTTCTGGACTGCCGCATAG
SEQ ID NO: 28 (DNA)
TTCTAATACGACTCACTATAG(N)₂₀GTTTTAGAGCTAGA (N can be any of A, T, G, and C)
SEQ ID NO: 29 (DNA sequence for miR-218 binding site)
ACATGGTTAGATCAAGCACAA
SEQ ID NO: 30 (RNA sequence for miR-218 binding site)
ACAUGGUUAGAUCAAGCACAA
SEQ ID NO: 31 (DNA sequence for miR-375 binding site)
GGTTTGTGCGAGGGGCTCGTCGC
SEQ ID NO: 32 (RNA sequence for miR-375 binding site)
GGUUUGUGCGAGGGGCUCGUCGC
SEQ ID NO: 33 (DNA, Cas 9 fwd)
CATGGCATGCGAATTCATGGACAAGAAGTACTCCATTGGGC
SEQ ID NO: 34 (DNA, Cas 9 reverse)
AAGCGAGCTCACTAGTTTAGTCTCCACCGAGCTGAGAG
SEQ ID NO: 35
CATGGCATGCGAATTCGCCACCATGGATTACAAAGACG
SEQ ID NO: 36
AAGCGAGCTCACTAGTTCAGGCACCGGGCTTGCG
SEQ ID NO: 37
AAGCGAGCTCACTAGTTTACTTGTACAGCTCGTCCATGCC
SEQ ID NO: 38
CATGGCATGCGAATTCGCCACCATGACTGCCATGG
SEQ ID NO: 39
AAGCGAGCTCACTAGTTCAGTCTGAGTCAGGCCCC
SEQ ID NO: 40
CATGGCATGCGAATTCGCCACCATGAAACGGACAGC
SEQ ID NO: 41
AAGCGAGCTCACTAGTTTATACCTTACGCTTCTTCTTTGGC
SEQ ID NO: 42
ATCTCTCCACTTCAGGTGGGT
SEQ ID NO: 43
TGTATTTGCTGGTGAAGTTGGT
SEQ ID NO: 44
AGAGAGGGTGTCAGGGAGGT
SEQ ID NO: 45
CTAAAAAGCCGCGAAGACAG
SEQ ID NO: 46
CTCACAGGCATTCACAAGGA
SEQ ID NO: 47
GCAGGAGTCACTGTCTGCAC
SEQ ID NO: 48
TTGTGGACCTGCATGTTTGT
SEQ ID NO: 49
CACAAACACTACAGAGGTTTTGTATTC
SEQ ID NO: 50
GCGTTTAATATAAGTGGAGGCG
SEQ ID NO: 51
CACCAAGGAGAACTTGGAGAAG
SEQ ID NO: 52
GGGCGAGGAGCTGTTCACCG
SEQ ID NO: 53
CATGGCGGCAGTCCAGACGA
SEQ ID NO: 54
GCCTTCACTAGCAAGGACGA
SEQ ID NO: 55
ACTCACGCTGGATAGCCTCC
SEQ ID NO: 56
GAAACAGCTCGTTGTACCGC

1. A gene editing system comprising
a first chemically modified RNA (modRNA) comprising a sequence encoding a CRISPR-associated (Cas) endonuclease or a base editor; and
a second modRNA comprising a sequence encoding a mutated p53 protein, wherein the mutated p53 protein inhibits a wild type p53 protein from binding to a target domain thereof.

2. (canceled)

3. (canceled)

4. The gene editing system of claim 1, wherein the first modRNA and the second modRNA each comprises a m7G(5)pppN1pN2p (CAP0) or m7G(5)pppN1mpNp (CAP1).

5. The gene editing system of claim 1, wherein the first modRNA and/or the second modRNA each comprises one or more N1-methyl-pseudouridine triphosphate (N1-methyl-pseudo-UTP).

6. The gene editing system of claim 5, wherein the uridines of the first modRNA and/or the second modRNA are N1-methyl-pseudo-UTPs.

7. (canceled)

8. (canceled)

9. (canceled)

10. The gene editing system of claim 1, wherein the first modRNA comprises a sequence at least 90% identical to SEQ ID NO: 1 and/or wherein the second modRNA comprises a sequence at least 90% identical to SEQ ID NO: 2.

11. (canceled)

12. The gene editing system of claim 1, wherein the base editor is an adenine base editor (ABE) or cytidine base editors (CBE).

13. The gene editing system of claim 12, wherein the sequence encoding the base editor is at least 90% identical to SEQ ID NO: 3.

14. (canceled)

15. (canceled)

16. (canceled)

17. A system for expressing a polynucleotide sequence specifically in a cell, said system comprising:
a regulatory RNA comprising a first nucleic acid sequence encoding a Cas6 endonuclease and a second nucleic acid sequence comprising a MicroRNA (miRNA) binding site, wherein miRNA is specifically expressed in the cell; and
a gene-of-interest (GOI) RNA comprising a Cas6 endonuclease targeting site and the polynucleotide sequence.

18. The system of claim 17, wherein the regulatory RNA and/or the GIO RNA are chemically modified.

19. The system of claim 18, wherein the chemically modified regulatory RNA and the chemically modified GIO RNA each comprises a m7G(5)pppN1pN2p (CAP0) or m7G(5)pppN1mpNp (CAP1).

20. The system of claim 18, wherein the chemically modified regulatory RNA and the chemically modified GIO RNA each comprises one or more N1-methyl-pseudouridine triphosphate (N1-methyl-pseudo-UTP).

21. The system of claim 20, wherein the uridines of the chemically modified regulatory RNA and the chemically modified GIO RNA are N1-methyl-pseudo-UTP.

22. (canceled)

23. (canceled)

24. The system of claim 17, wherein the first nucleic acid sequence encoding the Cas6 endonuclease is at least 90% identical to SEQ ID NO: 5 and/or wherein the miRNA binding site comprises a miRNA-206 binding site, a mir-218 binding site, or a miR-375 binding site.

25. The system of claim 17, wherein the miRNA binding site comprises a miRNA-206 binding site, a mir-218 binding site, or a miR-375 binding site.

26. The system of claim 17, wherein the miRNA binding site comprises a sequence at least 90% identical to SEQ ID NO: 6.

27. (canceled)

28. The system of claim 17.

29. The system of claim 28, wherein the pathogen is a virus or a bacterium.

30. The system of claim 29, wherein the virus is SARS-CoV-2.

31. (canceled)

32. (canceled)

33. (canceled)

34. (canceled)

35. A vaccine comprising the system of claim 17.

36. The vaccine of claim 35, wherein the vaccine is a SARS-CoV-2 vaccine.

37. (canceled)

38. A method of treating a genetic disorder or myocardial infarctions in a subject in need, comprising administering to the subject a therapeutically effective amount of the gene therapy composition comprising the system of claim 1.

39. The method of claim 38, wherein the genetic disorder comprises muscular dystrophy, hereditary neuropathy, Parkinson's disease, X-Linked cardiomyopathy, or cancer.

40. A method of modifying a gene in a cell, comprising introducing into the cell the system of claim 17.

41. A method for expressing a polynucleotide sequence specifically in a cell, comprising introducing into the cell the system of claim 17.

42. (canceled)


SEQUENCE LISTING (56 sequences)

SEQ ID NO: 1 - RNA, 4227 nt, synthetic construct
  source 1..4227: mol_type = other RNA
atggattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgtcctgctgctactaagaaagctggtcaagctaagaaaaagaaa

SEQ ID NO: 2 - RNA, 309 nt, synthetic construct
  source 1..309: mol_type = other RNA
atgactgccatggaggagtcacagtcggatatcagcctcaagagagcgctgcccacctgcacaagcgcctctcccccgcaaaagaaaaaaccacttgatggagagtatttcaccctcaagatccgcgggcgtaaacgcttcgagatgttccgggagctgaatgaggccttagagttaaaggatgcccatgctacagaggagtctggagacagcagggctcactccagctacctgaagaccaagaagggccagtctacttcccgccataaaaaaacaatggtcaagaaagtggggcctgactcagactga

SEQ ID NO: 3 - RNA, 4815 nt, synthetic construct
  source 1..4815: mol_type = other RNA
atgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggatggagaaattctaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtc

SEQ ID NO: 4 - RNA, 597 nt, synthetic construct
  source 1..597: mol_type = other RNA
tcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 5 - RNA, 567 nt, synthetic construct
  source 1..567: mol_type = other RNA
atgggtgatcattatctggatattcggctgaggcctgatccagagttcccacctgcgcagctgatgtctgtcctttttggcaaacttcatcaggccctggttgcccagggcggagatcggataggggtaagctttccagacctcgacgaaagccggagccgcctgggagaacgcctgcggatccacgcttctgccgacgatctgagagccttgctggcaaggccatggcttgaggggctccgggatcacctgcagtttggcgaacccgccgttgttccccacccaaccccttatcggcaggtgtctagagtgcaggccaaatctaatccagaacggctgcgacggcgactcatgcggcgacatgatcttagcgaggaagaggcccgaaaaagaatccctgataccgtggcccgcgcccttgacttgccttttgtcacactgcggtcccagagtacggggcagcatttcagacttttcattcgacacgggccactgcaagttaccgccgaagaaggaggctttacttgttatggactctccaagggaggtttcgtgccctggttttag

SEQ ID NO: 6 - RNA, 22 nt, synthetic construct
  source 1..22: mol_type = other RNA
ccacacacttccttacattcca

SEQ ID NO: 7 - RNA, 22 nt, synthetic construct
  source 1..22: mol_type = other RNA
gttcactgccgtataggcagct

SEQ ID NO: 8 - DNA, 4227 bp, synthetic construct
  source 1..4227: mol_type = other DNA
atggattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgtcctgctgctactaagaaagctggtcaagctaagaaaaagaaa

SEQ ID NO: 9 - DNA, 309 bp, synthetic construct
  source 1..309: mol_type = other DNA
atgactgccatggaggagtcacagtcggatatcagcctcaagagagcgctgcccacctgcacaagcgcctctcccccgcaaaagaaaaaaccacttgatggagagtatttcaccctcaagatccgcgggcgtaaacgcttcgagatgttccgggagctgaatgaggccttagagttaaaggatgcccatgctacagaggagtctggagacagcagggctcactccagctacctgaagaccaagaagggccagtctacttcccgccataaaaaaacaatggtcaagaaagtggggcctgactcagactga

SEQ ID NO: 10 - DNA, 4815 bp, synthetic construct
  source 1..4815: mol_type = other DNA
atgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggatggagaaattctaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtc

SEQ ID NO: 11 - DNA, 597 bp, synthetic construct
  source 1..597: mol_type = other DNA
tcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 12 - DNA, 567 bp, synthetic construct
  source 1..567: mol_type = other DNA
atgggtgatcattatctggatattcggctgaggcctgatccagagttcccacctgcgcagctgatgtctgtcctttttggcaaacttcatcaggccctggttgcccagggcggagatcggataggggtaagctttccagacctcgacgaaagccggagccgcctgggagaacgcctgcggatccacgcttctgccgacgatctgagagccttgctggcaaggccatggcttgaggggctccgggatcacctgcagtttggcgaacccgccgttgttccccacccaaccccttatcggcaggtgtctagagtgcaggccaaatctaatccagaacggctgcgacggcgactcatgcggcgacatgatcttagcgaggaagaggcccgaaaaagaatccctgataccgtggcccgcgcccttgacttgccttttgtcacactgcggtcccagagtacggggcagcatttcagacttttcattcgacacgggccactgcaagttaccgccgaagaaggaggctttacttgttatggactctccaagggaggtttcgtgccctggttttag

SEQ ID NO: 13 - DNA, 22 bp, synthetic construct
  source 1..22: mol_type = other DNA
ccacacacttccttacattcca

SEQ ID NO: 14 - DNA, 22 bp, synthetic construct
  source 1..22: mol_type = other DNA
gttcactgccgtataggcagct

SEQ ID NO: 15 - DNA, 11381 bp, synthetic construct
  source 1..11381: mol_type = other DNA
atcacctcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttgcttatgtaaaccagggcgcctataaaagagtgctgattttttgagtaaacttcaattccacaacacttttgtcttataccaactttccgtaccacttcctaccctcgtaaaggtaccgccaccatgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggatggagaaattctaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtcgctagcggcagcggcgccactaacttctccctgttgaaacaagcaggggatgtcgaagagaatcccgggccaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagccaaagaagaagcgtaaggtataagtacaagtaagaattccgatcatattcaataacccactagtagaccacctcccctgcgagctaagctggacagccaatgacgggtaagagagtgacatttttcactaacctaagacaggagggccgtcagagctactgcctaatccaaagacgggtaaaagtgataaaaatgtatcactccaacctaagacaggcgcagcttccgagggatttgagatccagacatgataagatacattgatgagtttggacaaaccaaaactagaatgcagtgaaaaaaatgccttatttgtgaaatttgtgatgctattgccttatttgtaaccattataagctgcaataaacaagtttgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaaggtcgacttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttagcagtgaaaaaaatgctttatttgtgaaatttgtgatgctattgctttatttgtaaccattataagctgcaataaacaagttaactaaacgggccctctagatcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatctcgagcctagggccgggattctcctccacgtcaccgcatgttagaagacttcctctgccctctccgctgcccccggggagcatgtcaaggtcaaaatcgtcaagagcgtcagcaggcagcatatcaaggtcaaagtcgtcaagggcatcggctgggagcatgtctaagtcaaaatcgtcaagggcgtcggtcggcccgccgctttcgcactttagctgtttctccaggccacatatgattagttccaggccgaaaaggaaggcaggttcggctccctgccggtcgaacagctcaattgcttgtttcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggtatgacttggcgttgttccgcagaaagtcttgccatgactcgccttccagggggcaggagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcagagtttatgactttgctcttgtccagtctagacatcttatcgtcatcgtctttgtaatccatggtggcggatcccgcgtcacgacacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccactcgagtggaatt

SEQ ID NO: 16 - DNA, 43 bp, synthetic construct
  source 1..43: mol_type = other DNA
gcctgcctagtggaccagagccttcgtctggactgccgccatg

SEQ ID NO: 17 - DNA, 25 bp, synthetic construct
  source 1..25: mol_type = other DNA
gcctgcctagtggactgccgccatg

SEQ ID NO: 18 - DNA, 51 bp, synthetic construct
  source 1..51: mol_type = other DNA
gcctgcctagtggaccagagccttgagaattctcattggactgccgccatg

SEQ ID NO: 19 - DNA, 40 bp, synthetic construct
  source 1..40: mol_type = other DNA
gcctgcctagtggaccagagccttctggactgccgccatg

SEQ ID NO: 20 - DNA, 25 bp, synthetic construct
  source 1..25: mol_type = other DNA
gcctgcctagtggaccagagccatg

SEQ ID NO: 21 - DNA, 44 bp, synthetic construct
  source 1..44: mol_type = other DNA
gcctgcctagtggaccagagccttcgttctggactgccgccatg

SEQ ID NO: 22 - DNA, 23 bp, synthetic construct
  source 1..23: mol_type = other DNA
ggctatccagcgtgagtctctcc

SEQ ID NO: 23 - DNA, 23 bp, synthetic construct
  source 1..23: mol_type = other DNA
ggctatccagcgcgagcctctcc

SEQ ID NO: 24 - DNA, 23 bp, synthetic construct
  source 1..23: mol_type = other DNA
ggctatccagcgcgagtctctcc

SEQ ID NO: 25 - DNA, 53 bp, synthetic construct
  source 1..53: mol_type = other DNA
tgaatgacaccatgcagccccgcccatgggccctcgtctggactgcctctttc

SEQ ID NO: 26 - DNA, 53 bp, synthetic construct
  source 1..53: mol_type = other DNA
cagagctgcagtgcagacgagggttgggcacctcagagctgcagtgcagacag

SEQ ID NO: 27 - DNA, 53 bp, synthetic construct
  source 1..53: mol_type = other DNA
ctttttatatgttgtgtctctgatgattttccctcttctggactgccgcatag

SEQ ID NO: 28 - DNA, 55 bp, synthetic construct
  source 1..55: mol_type = other DNA
  misc_feature 22..41: note = n can be any of a, t, g, and c
ttctaatacgactcactatagnnnnnnnnnnnnnnnnnnnngttttagagctaga

SEQ ID NO: 29 - DNA, 21 bp, synthetic construct
  source 1..21: mol_type = other DNA
acatggttagatcaagcacaa

SEQ ID NO: 30 - RNA, 21 nt, synthetic construct
  source 1..21: mol_type = other RNA
acatggttagatcaagcacaa

SEQ ID NO: 31 - DNA, 23 bp, synthetic construct
  source 1..23: mol_type = other DNA
ggtttgtgcgaggggctcgtcgc

SEQ ID NO: 32 - RNA, 23 nt, synthetic construct
  source 1..23: mol_type = other RNA
ggtttgtgcgaggggctcgtcgc

SEQ ID NO: 33 - DNA, 41 bp, synthetic construct
  source 1..41: mol_type = other DNA
catggcatgcgaattcatggacaagaagtactccattgggc

SEQ ID NO: 34 - DNA, 38 bp, synthetic construct
  source 1..38: mol_type = other DNA
aagcgagctcactagtttagtctccaccgagctgagag

SEQ ID NO: 35 - DNA, 38 bp, synthetic construct
  source 1..38: mol_type = other DNA
catggcatgcgaattcgccaccatggattacaaagacg

SEQ ID NO: 36 - DNA, 34 bp, synthetic construct
  source 1..34: mol_type = other DNA
aagcgagctcactagttcaggcaccgggcttgcg

SEQ ID NO: 37 - DNA, 40 bp, synthetic construct
  source 1..40: mol_type = other DNA
aagcgagctcactagtttacttgtacagctcgtccatgcc

SEQ ID NO: 38 - DNA, 35 bp, synthetic construct
  source 1..35: mol_type = other DNA
catggcatgcgaattcgccaccatgactgccatgg

SEQ ID NO: 39 - DNA, 35 bp, synthetic construct
  source 1..35: mol_type = other DNA
aagcgagctcactagttcagtctgagtcaggcccc

SEQ ID NO: 40 - DNA, 36 bp, synthetic construct
  source 1..36: mol_type = other DNA
catggcatgcgaattcgccaccatgaaacggacagc

SEQ ID NO: 41 - DNA, 41 bp, synthetic construct
  source 1..41: mol_type = other DNA
aagcgagctcactagtttataccttacgcttcttctttggc

SEQ ID NO: 42 - DNA, 21 bp, synthetic construct
  source 1..21: mol_type = other DNA
atctctccacttcaggtgggt

SEQ ID NO: 43 - DNA, 22 bp, synthetic construct
  source 1..22: mol_type = other DNA
tgtatttgctggtgaagttggt

SEQ ID NO: 44 - DNA, 20 bp, synthetic construct
  source 1..20: mol_type = other DNA
agagagggtgtcagggaggt

SEQ ID NO: 45 - DNA, 20 bp, synthetic construct
  source 1..20: mol_type = other DNA
ctaaaaagccgcgaagacag

SEQ ID NO: 46 - DNA, 20 bp, synthetic construct
  source 1..20: mol_type = other DNA
ctcacaggcattcacaagga

SEQ ID NO: 47 - DNA, 20 bp, synthetic construct
  source 1..20: mol_type = other DNA
gcaggagtcactgtctgcac

SEQ ID NO: 48 - DNA, 20 bp, synthetic construct
  source 1..20: mol_type = other DNA
ttgtggacctgcatgtttgt

SEQ ID NO: 49 - DNA, 27 bp, synthetic construct
  source 1..27: mol_type = other DNA
cacaaacactacagaggttttgtattc

SEQ ID NO: 50 - DNA, 22 bp, synthetic construct
  source 1..22: mol_type = other DNA
gcgtttaatataagtggaggcg

SEQ ID NO: 51 - DNA, 22 bp, synthetic construct
  source 1..22: mol_type = other DNA
caccaaggagaacttggagaag

SEQ ID NO: 52 - DNA, 20 bp, synthetic construct
  source 1..20: mol_type = other DNA
gggcgaggagctgttcaccg

SEQ ID NO: 53 - DNA, 20 bp, synthetic construct
  source 1..20: mol_type = other DNA
catggcggcagtccagacga

SEQ ID NO: 54 - DNA, 20 bp, synthetic construct
  source 1..20: mol_type = other DNA
gccttcactagcaaggacga

SEQ ID NO: 55 - DNA, 20 bp, synthetic construct
  source 1..20: mol_type = other DNA
actcacgctggatagcctcc

SEQ ID NO: 56 - DNA, 20 bp, synthetic construct
  source 1..20: mol_type = other DNA
gaaacagctcgttgtaccgc